(19) United States
(12) Patent Application Publication
(10) Pub. No.: US 2026/0364259 A1
(43) Pub. Date: Nov. 19, 2026

(54) INJECTION DEVICE

(71) Applicant: I D & E PTY LTD., New South Wales (AU)

(72) Inventors: RICHARD SOKOLOV, New South Wales (AU); HUW UMBERTO WALLIS, New South Wales (AU); ADRIANUS JOHANNES BOVENDEERT, New South Wales (AU); PHILIP JOHAN ALEXANDER FRANK VAN GRIETHUIJSEN, New South Wales (AU); NIKOLAI CHIKOVSKII, New South Wales (AU)

(73) Assignee: I D & E PTY LTD., New South Wales (AU)

(21) Appl. No.: 19/477,342

(22) PCT Filed: Apr. 23, 2024
(86) PCT No.: PCT/AU2024/050387

§ 371 Date: Oct. 21, 2025

(30) Foreign Application Priority Data
Apr. 24, 2023 (AU) 2023202488
Mar. 4, 2024 (AU) 2024900553

(51) Int. Cl. A61M 5/20; A61M 5/31; A61M 5/32

(52) U.S. Cl. CPC A61M 5/2033; A61M 5/3146; A61M 5/3243; A61M 2005/3126

(57) ABSTRACT

The present invention relates to an injection device comprising an elongated housing having a proximal end and a distal end, the proximal end comprising an actuation mechanism, wherein: said elongated housing defines an inner lumen between said proximal and distal ends which is adapted to receive and hold a formulation chamber relative to said actuation mechanism, said formulation chamber also having corresponding proximal and distal ends, and having a stopper between its proximal and distal ends for retaining formulation in said formulation chamber; said actuation mechanism having corresponding proximal and distal ends, and comprises an actuation housing with a proximal end, and a distal end adapted for engaging said elongated housing, wherein said actuation mechanism includes a plunger, the distal of said plunger engaging said stopper or being adapted to engage said stopper, said plunger being in communication with at least one driving spring which biases the plunger distally when the actuation mechanism is primed for injection, wherein said plunger is held in position against said bias by at least one retention arm or toggle having at least one first surface adapted to engage an opposed complementary second surface to hold said plunger in a first unactuated position when the actuation mechanism is primed for injection, the first and second surfaces being held in an engaged position by a restrainer which translates from an unactuated position or configuration to an actuated position or configuration when triggered, thereby allowing uncoupling of the engagement between the first and second surfaces and release of the plunger to move distally. The present invention also relates to needle hubs, actuation mechanisms, dosing mechanisms and assembled elongated housings for use with such injection devices, as well as methods of using such devices.

### CROSS-REFERENCE TO RELATED APPLICATION(S)

This application is a U.S. National Stage of International Patent Application No. PCT/AU2024/050387, entitled INJECTION DEVICE, filed on Apr. 23, 2024, which claims priority to Australian Application Nos. 2024900553, filed on Mar. 4, 2024, and 2023202488, filed on Apr. 24, 2023, the disclosures of which are incorporated herein by reference in their entirety.

### FIELD OF THE INVENTION

The present invention relates to improvements in injection devices for delivering active agents to patients, particularly but not exclusively intravitreal injection devices.

### BACKGROUND TO THE INVENTION

A great number of different injection devices exist and have been proposed over the decades. The most basic of such devices is the basic syringe with needle, barrel and plunger, with the needle being optionally covered with a cap, and the syringe potentially also being pre-filled. The needle could be an integral part of the syringe (i.e., being moulded with the barrel), or could be a separate part and be attached via a tight or nested fit, or via a snap or screw-type mechanism such as a Luer lock attachment.

Such injection devices are potentially dangerous due to exposure of the patient and the injector to the needle during administration. Care is also needed to inject the correct dose of active agent to a patient and at the correct depth for efficacy and to avoid damage to other structures, such as in intravitreal injections.

More recently devices with protective sheaths have been developed which keep the needle always shielded when not in use—the sheath is biased towards a shielding position and is pushed out of the way as the needle is pushed towards, and into the intended subject. Such sheaths, however, typically are not locked into place, so pushing of the used injection device against another surface may result in puncturing that surface, which may be a human surface, as well.

Automatic injection devices have also been described which automatically dispense a predetermined amount of agent when pushed against a subject, and which may also have a biased sheath to ensure shielding of the needle when not injecting. Such injection devices are, however, generally designed to dispense one particular amount, regardless of the recipient's age, size, health or other physiological parameter (such as the Epi-Pen®), and typically dispense the agent in an abrupt short burst, which may be undesirable in many circumstances, such as when, for example, injecting into the eye.

Motorised injection devices have also been described which dispense desired amounts of agent, optionally as step-wise sequential injections. Such injection devices are, however, very complex and expensive, and are unsuitable for single use or disposable injection procedures.

Exemplary injection devices for intraocular injection, including intravitreal injection are known. Intravitreal injection in particular can be complex, as the desired injection site is in the Pars plana, about 3-4 mm behind the sclerocorneal limbus. In addition, due to the sensitivity and delicacy of the injection area, fine modulation of the force needed to inject the medicine is necessary to reduce the risk for adverse effects. However, as many of the medicinal products typically utilized in ophthalmic procedures are biologic products, such products may be of a higher viscosity than other drug products typically used with exemplary autoinjector products, which further complicates dosing and delivery. As such, traditionally such injections have been performed by a physician using a standard syringe and a measuring device (placed on the eye, surrounding the cornea, and providing a spaced hole for placing the needle through).

Exemplary, devices designed specifically for intravitreal injection typically utilize a spacing measuring device at the injection end of the device to correctly position the needle over the Pars plana. However, such devices generally: do not sufficiently ensure the correct injection depth (which is important in intravitreal injection); do not lock a sheath over the needle once used or protect the needle fluid path and injection site from contamination; do not lend themselves to easily adjustable, accurate and automatic dosing; are complex, expensive and do not readily lend themselves to one-off or disposable injection procedures, or a combination of such disadvantages.

It is an object of the present invention to provide an improved automatic injection device and system which addresses these challenges, including the provision of accurate, adjustable dosing and modulation of injection and delivery force to reduce risk to patients, improve clinical workflows, and improve procedure effectiveness.

### SUMMARY OF THE INVENTION

According to a first embodiment of the present invention there is provided an injection device comprising an elongated housing having a proximal end and a distal end, the proximal end comprising an actuation mechanism, wherein: the elongated housing defines an inner lumen between the proximal and distal ends which is adapted to receive and hold a formulation chamber relative to the actuation mechanism, the formulation chamber also having corresponding proximal and distal ends, and having a stopper between its proximal and distal ends for retaining formulation in the formulation chamber; the actuation mechanism having corresponding proximal and distal ends, and comprises an actuation housing with a proximal end, and a distal end adapted for engaging the elongated housing, wherein the actuation mechanism includes a plunger, the distal end of the plunger engaging the stopper or being adapted to engage the stopper, the plunger being in communication with at least one driving spring which biases the plunger distally when the actuation mechanism is primed for injection; wherein the plunger is held in position against the bias by at least one retention arm or toggle having at least one first surface adapted to engage an opposed complementary second surface to hold the plunger in a first unactuated position when the actuation mechanism is primed for injection, the first and second surfaces being held in an engaged position by a restrainer which translates from an unactuated position or configuration to an actuated position or configuration when triggered, thereby allowing uncoupling of the engagement between the first and second surfaces and release of the plunger to move distally.

In certain embodiments the restrainer may translate from the unactuated position or configuration to the actuated position or configuration by a trigger mechanism activated when a needle connected to the injection device reaches a desired injection depth and may, for example, comprise a mechanism that moves the restrainer proximally or distally as the needle is inserted into a subject to release said retention arm or toggle as the needle reaches a desired depth of insertion. However, it is also contemplated that the restrainer may be translated from a retention arm or toggle-restraining position or configuration to a releasing position or configuration by a mechanism activated by a button which is pressed or slid when the needle is at the desired injection depth. Alternatively, once the restrainer is translated from a retention arm or toggle-restraining position or configuration to a releasing position or configuration, the retention arm or toggle may still remain in its unactuated position until a mechanism is activated by a button which is pressed or slid by an operator.

In a particular embodiment, the elongated housing also comprises a sleeve which is at least proximally slidable between the elongated housing and the formulation chamber, and which slides proximally as a needle connected to the injection device is inserted into a subject. In particular aspects the sleeve is adapted to engage the restrainer such that proximal movement of the sleeve causes proximal movement of the restrainer, and wherein operatively, insertion of a needle connected to the injection device into a surface results in the sleeve sliding proximally and, as a result, the restrainer moves proximally to the actuated position or configuration, although it is also envisaged that proximal movement of the sleeve may move the restrainer distally (for example, by leveraging or other opposing/complementary mechanisms). In certain aspects, movement of the restrainer to a retention arm or toggle-releasing position results in release of the retention arm or toggle, distal movement of the plunger and delivery of formulation to a subject. However, it is also contemplated that the plunger may still be restrained from distal movement despite the restrainer being in a releasing position/configuration until another mechanism is triggered to a release mode by, for example, a manually activated button. The sleeve may be between the elongated housing and a formulation chamber when received therein or be external to the elongated housing, or a combination thereof.

In a particular aspect, the restrainer is in the unactuated position when distal and in the actuated position when proximal, so that operatively, the restrainer moves proximally as the sleeve is slid proximally relative to the elongated housing, thereby releasing the retention arm or toggle to uncouple the first and second surfaces allowing said driving spring to drive the plunger and the stopper distally.

In certain aspects the retention arm or toggle is a retention toggle which has distal and proximal ends, and is connected to the plunger by a pivot point located between said distal and proximal ends. In certain aspects the first surface of the toggle may be in the form of an outwardly directed ledge adapted to engage an opposed complementary second surface in the form of an inwardly directed ledge on or connected to the actuation housing, the proximal end of the toggle including a surface adapted to engage a complementary surface of the restrainer. In certain aspects the outwardly directed ledge may be positioned between the pivot point and the proximal end of the toggle.

In certain aspects the restrainer may be connected to, or form part of the proximal end of the sleeve. In other aspects the restrainer may be separate to the sleeve.

In certain aspects, at least when in use, the distal end of the injection device may comprise a sheath enshrouding at least a distal portion of a needle assembly in fluid communication with the formulation chamber, the sheath being either an integral part of the sleeve or being adapted to engage the distal end of the sleeve, and being adapted to slide between the elongated housing and the formulation chamber. The sheath may be at least proximally slidable with respect to the needle so as to unsheath the needle during insertion of said needle into a subject during injection and, in the process, moves the sleeve proximally, resulting in proximal movement of the restrainer to a retention arm or toggle-releasing position, allowing release of the plunger.

In certain aspects the sleeve and sheath are held in a needle enshrouding position by a second spring stressed between the actuation housing or the plunger and the sleeve, which is adapted to engage the second spring.

In other aspects the sheath comprises a spring or other suitable biasing means of its own stressed between a distal portion of the sheath and the needle assembly to ensure the sheath covers the needle tip.

In certain aspects the retention arm or toggle, once released, is adapted to stop the sleeve and sheath from proximal movement once the sleeve and sheath have returned to a needle-enshrouding position.

The at least one retention arm or toggle may, in certain aspects, comprise at least one biased arm, said at least one biased arm being connected at one end to at least one of the proximal end of the actuation mechanism housing or, in a preferred embodiment, the proximal end of the plunger, the other end of said at least one biased arm comprising an inwardly directed ledge adapted to engage a complementary outwardly directed ledge on at least one of the plunger or, in a preferred embodiment, the actuation mechanism housing, said outwardly directed ledge being optionally formed as a groove in or a ridge on said plunger.

This configuration of the injection device actuation mechanism is such that the force to move the restrainer proximally to release the retention arm or toggle can be lowered significantly compared to mechanisms where the retention arm or toggle are biased inwardly against the plunger. According to certain aspects of the present invention, the triggering force required to allow uncoupling of the engagement between the first and second surfaces and release of the plunger to move distally is inversely proportional to a value a/b, wherein a is the distance between the proximal end of the toggle and the pivot point and b is the adaxial extension distance between the edge of the first surface and the pivot point, allowing regulation of the triggering force by regulation of the value a/b.

The retention arm or toggle may comprise biasing means as an integral component, or may be biased by biasing means attached to a separate component of the actuation mechanism. In certain aspects the at least one retention arm or toggle is biased by integral biasing means which, when the device is primed for injection, apply force against and away from the plunger. In other aspects the at least one retention arm or toggle is biased by biasing means connected to the plunger and which, when the device is primed for injection, apply force against the retention arm or toggle, away from the plunger.

In certain aspects the actuation mechanism housing and the elongated housing may be detachable from one another to allow placement, removal or replacement of formulation chamber(s) into or from said elongated housing.

In certain aspects the formulation chamber is pre-filled.

In other aspects the formulation chamber is vacuum or voidless filled, in which case engagement of the plunger with the stopper and placement of the formulation chamber in the elongated housing and connection of the actuation housing to the elongated housing results in positive pressure within the formulation chamber. This configuration provides the advantage that when a needle is placed in fluid communication with the distal end of the formulation chamber, the positive pressure within the formulation chamber results in a small amount of fluid being expelled from the formulation chamber through the needle, the needle being thereby filled. This automatically primes the device with formulation and further prevents air or other fluid or contaminants from entering the formulation chamber.

In certain aspects a needle assembly is attached to the formulation chamber and the distal end of the formulation chamber is adapted to have the needle assembly attached thereto.

In other aspects a needle assembly is attached to the elongated housing and the distal end of the elongated housing is adapted to have a needle assembly attached thereto and allow fluid communication between the formulation chamber and the needle. This requires bridging of the elongated housing between sleeve leaves, such that the sleeve is slidable between the outer portion of the elongated housing and the inner needle assembly-engaging portion of the elongated housing. In particular aspects, the elongated housing comprises a sleeve which is at least proximally slidable between the elongated housing and the formulation chamber and is adapted to engage the restrainer such that proximal movement of the sleeve causes proximal movement of the restrainer and wherein, operatively, the distal end of the elongated housing comprises a needle assembly having a sheath enshrouding at least a distal portion of a needle in fluid communication with said formulation chamber, wherein the sheath is at least proximally slidable with respect to the needle so as to unsheath the needle during insertion of the needle into a subject, resulting in proximal sliding of the sleeve and proximal movement of the restrainer to the actuated position.

In certain aspects the needle assembly is attached to the distal end of said formulation chamber or said elongated housing by means of a tight fit, snap, screw or a Luer lock mechanism.

In alternative aspects the needle may form an integral part of at least the formulation chamber or be an integral part of the elongated housing.

In a specific embodiment, in an injection device according to the invention, the elongated housing comprises a sleeve which is at least adapted to slide proximally between the elongated housing and the formulation chamber and is adapted to engage the restrainer such that proximal movement of the sleeve causes proximal movement of the restrainer; the distal end of said elongated housing is connected to or is adapted to be connected to a needle assembly comprising a sheath, wherein said sheath is at least proximally slidable with respect to the needle so as to unsheath the needle during insertion of said needle into a subject and so as to slide said sleeve proximally, which results in proximal movement of said restrainer from the first unactuated position to the second actuated position; the plunger is held in position against said stress by a toggle with distal and proximal ends and connected to the plunger by a pivot point located between said distal and proximal ends and wherein said first surface is in the form of an outwardly directed ledge positioned between the pivot point and the proximal end and adapted to engage said opposed complementary second surface provided in the form of a complementary inwardly directed ledge on or connected to the actuation housing, the proximal end of the toggle comprising a surface adapted to engage a complementary surface of the restrainer when in the unactuated position.

According to another embodiment of the present invention, there is provided an injection device comprising an elongated housing having a proximal end and a distal end, the proximal end comprising an actuation mechanism, wherein: the elongated housing defines an inner lumen between the proximal and distal ends which includes or is adapted to receive and hold a formulation chamber relative to said actuation mechanism, the formulation chamber also having corresponding proximal and distal ends, and having a stopper between its proximal and distal ends for retaining formulation in said formulation chamber; the actuation mechanism has corresponding proximal and distal ends, and comprises an actuation housing with a proximal end, and a distal end adapted for engaging said elongated housing, wherein the actuation mechanism includes a plunger, the distal end of the plunger engaging the stopper or being adapted to engage said stopper, and the plunger being in communication with at least one driving spring which biases the plunger distally when the actuation mechanism is primed for injection, wherein the plunger is held in position against the bias by a toggle which has distal and proximal ends, and is connected to the plunger by a pivot point located between the distal and proximal ends, the toggle having an outwardly directed ledge positioned between the pivot point and the proximal end and adapted to engage an opposed inwardly directed ledge on or connected to the actuation housing to hold the plunger in a first unactuated position when the actuation mechanism is primed for injection, the proximal end of the toggle including a surface adapted to engage a complementary surface of a restrainer to hold the toggle in an unactuated position and thereby keep the outwardly directed ledge and opposed inwardly directed ledge in an engaged position, wherein the restrainer translates from an unactuated position or configuration to an actuated position or configuration when triggered, thereby allowing the toggle to deflect to an actuated position thereby uncoupling of the engagement between the outwardly directed ledge and opposed inwardly directed ledge and release of the plunger to move distally; the elongated housing comprises a sleeve which is at least adapted to slide proximally between the elongated housing and the formulation chamber and is adapted to engage the restrainer such that proximal movement of the sleeve causes proximal movement of the restrainer; and the distal end of the elongated housing is connected to or is adapted to be connected to a needle assembly comprising a sheath, the sheath being either an integral part of said sleeve or being adapted to engage the distal end of the sleeve, wherein the sheath is at least proximally slidable with respect to the needle so as to unsheath the needle during insertion of the needle into a subject and so as to slide the sleeve proximally, which results in proximal movement of the restrainer from the unactuated position to the actuated position, uncoupling of the engagement between the outwardly directed ledge and opposed inwardly directed ledge, and release of the plunger to move distally.

In certain aspects the actuation housing comprises a dosing mechanism which is adjustably coupled to the plunger at the proximal end of the actuation housing, such that a distance between the dosing mechanism and the actuation housing, and therefore an allowable travel distance of the plunger, can be set to a specified value, so that a selected volume of formulation may be operatively dispensed.

In alternative aspects the plunger has a proximal end that extends proximally beyond the actuation housing and engages with a cap or knob via adjustable means, such that a distance between the cap or knob and the actuation housing, and therefore the allowable travel distance of the plunger is controlled, so that operatively adjustment of the cap allows delivery of a selected volume of formulation. In one specific aspect the dosing mechanism comprises a first internal knob, a second, external dose setting knob, and a dose delivery housing adapted to sit on the proximal end of the actuation housing, wherein the proximal end of the plunger engages with the first knob via a thread with axial:longitudinal pitch X, the first knob engages with the second knob axially, but not longitudinally, and the second external dose setting knob engages with the dose delivery housing via a thread with axial:longitudinal pitch Y, wherein pitch Y is greater than pitch X, such that axial rotation of the second external dose setting knob results in the same degree of axial rotation as the first knob, but greater longitudinal travel of the second external dose setting knob compared to the first knob.

In certain embodiments of injection devices according to the present invention, the driving spring is positioned between the plunger and the proximal end of the actuation mechanism housing.

In other embodiments the driving spring may be offset compared to the plunger and biases the plunger indirectly.

In certain specific embodiments, an injection device according to the present invention is for intravitreal injection and comprises a sheath enshrouding at least a distal portion of a needle assembly in fluid communication with said formulation chamber, the sheath being either an integral part of said sleeve or being adapted to engage the distal end of the sleeve and being adapted to slide between the elongated housing and the formulation chamber, and wherein the sheath is at least proximally slidable with respect to the needle so as to unsheath the needle during insertion of the needle into a subject and so as to push the sleeve, resulting in proximal movement of the sleeve against the restrainer and movement of the restrainer to a retention arm or toggle-releasing position, wherein the sheath has a distal eye-contacting surface.

In certain aspects the eye-contacting surface has an outer perimeter that defines a circle or modified circle, said surface being annular or in the form of a perforated concave disc, discoid or 3-dimensional annulus surface, with the needle defining the centre of said circle, although other shapes may be used. Where the eye-contacting surface is annular or in the form of a perforated concave disc, discoid or 3-dimensional annulus surface, the diameter of the circle may be between 4 to 10 mm, more preferably between 6 mm and 8 mm, so as to provide a ready measurement of 2-5, preferably 3-4 mm from the limbus to the Pars plana, to ensure injection in the vitreous body of the eye. Where other eye-contacting surface shapes are used, this should still provide at least one eye-surface contacting edge that is 2-5 mm, preferably 3-4 mm from the needle to allow for correct positioning of the needle for intravitreal injection.

In certain aspects the sheath has an external pad which is substantially coterminous with the eye-contacting surface for applying pressure next to the injection site after injection. The external pad may be a peripheral extension to the perimeter of the eye-contacting surface, or may extend from the external surface of the sheath.

According to certain embodiments, injection devices according to the present invention may be for single use or for carrying out multiple injections, either of the same formulation or different formulations. Where the devices are for multiple injections, the needle hubs may be replaceable, the formulation chambers may be replaceable, or assembled elongated housings, complete with formulation chamber and needle assembly (typically also with a cap) may be replaceable. Where the devices are for single use, or the assembled actuation housing with formulation chamber is for single or limited multiple use, the formulation chamber may be integrally formed with the elongated housing. Where the devices are for single use the needle may be integral with the elongated housing as well or it could be part of a separate replaceable needle assembly or hub. In certain aspects the elongated housing is adapted to receive pre-filled formulation chambers, where a needle assembly is integrally formed with the syringe body, or where the elongated housing is adapted to have a needle assembly or needle hub attached to it such that the needle is in fluid communication with the formulation chamber.

According to another embodiment of the present invention there is provided a needle hub adapted to be attached to an injection device according to any of the above described injection device embodiments and comprising a needle and a body with the injection end of the needle protruding distally from it, the proximal end of the needle hub being adapted to connect to the engagement so that the needle can operatively communicate with the formulation chamber, the needle hub also including a sheath enshrouding the distal needle and main body, the proximal end of the sheath being operatively adapted, once the hub is connected to the injection device, to: retain the main body of the needle assembly within the sheath; to engage a sleeve of said injection device; and to fit and slide relative to the injection device. The body of the needle assembly may be integrally formed or overmoulded, or may be a separably or permanently attached component. The body may further have a proximal end having a proximal end of the needle protruding proximally from it and a main body with the injection end of the needle protruding distally from it, the proximal end of the body being adapted for engagement with the elongated housing or the formulation chamber, optionally via a snap, tight fit, screw or Luer lock mechanism. The needle hub may optionally also comprise a cap which fully enshrouds at least the distal end of said needle hub.

According to an alternative needle hub embodiment, there is provided a needle hub for attachment to an injection device according to any of the above described injection device embodiments and comprising a main body having a proximal body and a distal body, the main body being either connected to a member adapted to engage the distal end of the elongated housing or formulation chamber or having a proximal end adapted to engage the distal end of the elongated housing or formulation chamber, the proximal body having a proximal needle extending proximally from it, the distal body comprising a distal needle extending distally from it, the proximal and distal bodies being adapted to connect together with a filter enclosed between them and between the two needles, the needle hub also comprising a sheath enshrouding the needle and main body, the proximal end of the sheath being adapted to: retain the main body of a needle assembly within the sheath when not in use; to engage the sleeve of said injection device; and to fit and slide within the elongated housing. Such an embodiment allows for avoidance of microparticulate matter that may be present in the formulation in the formulation chamber and/or microparticulate matter that may arise as a result of the proximal needle piercing the distal seal on the formulation chamber. The proximal and distal needles may be of the same gauge (outer diameter) or may be different, with the proximal needle having a greater or smaller gauge than the distal needle. However, because the proximal needle is not involved in the injection, and is therefore not the subject of limitations for comfortable intravitreal injection, it may be of any desired gauge, whereas where the needle hub is for intravitreal injection, the distal needle may preferably be no larger than gauge 25 (approx. 0.5 mm outer diameter) or finer, preferably gauge 27 (approx. 0.4 mm outer diameter) or finer. The needle hub may optionally also comprise a cap which fully enshrouds at least the distal end of said needle hub, and typically will comprise such a cap when distributed. Optionally the cap also includes means to ensure that the cap does not rotate axially with respect to said main body when connecting the needle hub to said injection device.

According to certain aspects the needle hub is for intravitreal injection and has a distal eye-contacting surface, as described earlier. In certain aspects the sheath is substantially cylindrical for most of its length, with a frustoconical distal end.

The needle hub may comprise a sheath which has an external pad which is substantially coterminous with the eye-contacting surface for applying pressure next to the injection site after injection. The external pad may be a peripheral extension to the perimeter of the eye-contacting surface, or may extend from the external surface of the sheath.

The sheath may also comprise features that inhibit axial rotation of the sheath relative to the cap while securing the needle hub to an injection device according to the present invention.

According to another embodiment of the present invention there is provided a needle hub adapted to be attached to an injection device, the injection device including a formulation chamber and an engagement allowing connection to a formulation chamber, the needle hub comprising a needle and a body with the injection end of the needle protruding distally from it, the proximal end of the needle hub being adapted to connect to the engagement so that the needle can operatively communicate with the formulation chamber, the needle hub also including a sheath enshrouding the distal needle and main body, the proximal end of the sheath being operatively adapted, once the hub is connected to the injection device, to: retain the main body of the needle assembly within the sheath; to engage a sleeve of said injection device; and to fit and slide relative to the injection device, said needle hub optionally also comprising a cap which fully enshrouds at least the distal end of said needle hub.

According to another embodiment of the present invention there is provided an actuator mechanism for an injection device, the mechanism having a plunger, the distal end of the plunger being adapted to engaging a stopper, and the plunger being in communication with a driving spring which biases the plunger distally when the actuation mechanism is primed for injection, wherein the plunger is held in position against said stress by a retention arm or toggle having at least one first surface adapted to engage an opposed complementary second surface to hold said plunger in an unactuated position when the actuation mechanism is primed for injection, the first and second surfaces being held in an engaged position by a restrainer which translates from an unactuated position or configuration to an actuated position or configuration when triggered, thereby allowing uncoupling of the engagement between the first and second surfaces and release of the plunger to move distally.

According to another embodiment of the present invention there is provided an elongated housing assembly for use with any one of the above described injection device embodiments and comprising proximal and distal ends and which defines an inner lumen between said proximal and distal ends which includes or is adapted to receive and hold a formulation chamber relative to said actuation mechanism, said formulation chamber also having corresponding proximal and distal ends, and having a stopper between its proximal and distal ends for retaining formulation in said formulation chamber, and wherein said elongated housing also comprises a sleeve which is at least proximally slidable between the elongated housing and the formulation chamber and is adapted to engage the restrainer of the actuation housing, and wherein insertion of a needle connected to the elongated housing into a subject results in the sleeve sliding proximally.

According to another embodiment of the present invention there is provided a dose delivery mechanism for an injection device, the injection device comprising a body with a distal end which, operatively, has a needle assembly attached thereto and a proximal end comprising an actuation mechanism with a plunger with distal and proximal ends, the injection device further including a formulation chamber also with distal and proximal ends and including a stopper between its distal and proximal ends and formulation between the stopper and the distal end of the formulation chamber, wherein the distal end of the plunger is adapted to engage the stopper, and wherein said dose delivery mechanism is adjustably coupled to the proximal end of the plunger such that a distance between the dosing mechanism and the device body, and therefore an allowable travel distance of the plunger, can be set to a specified value to set a selected volume of formulation for delivery.

According to another embodiment of the present invention there is provided a method for operating an injection device, the injection device having an elongated housing having a proximal end and a distal end, the proximal end having an actuation mechanism and the distal end having or being adapted to have a needle attached thereto, the injection device further including a formulation chamber including a stopper; the actuation mechanism including a plunger, the distal end of the plunger being adapted to engage the stopper, the plunger being biased towards the distal end when the actuation mechanism is primed for injection, the plunger being held in position against the bias by a retention arm or toggle, so as to hold the plunger in an unactuated position when the actuation mechanism is primed for injection, the plunger being held in an engaged position by a restrainer which is adapted to translate from an unactuated position or configuration to an actuated position or configuration when triggered; the method comprising bringing the device into an operative position; triggering the restrainer to release the plunger from the engaged position, the plunger then moving distally to deliver formulation from the formulation chamber.

Accordingly, suitable implementations of the present invention provide one or more of reduced trigger force, more accurate injection depth, accurate and adjustable dosing, protection of the needle and/or fluidic pathway of the device, including pre-injection priming and post-injection locked needle protection, correct positioning of injection (for intravitreal injection), dose delivery confirmation, use of pre-filled syringes or cartridges, automatic priming, or any combination thereof.

### BRIEF DESCRIPTION OF THE DRAWINGS

Illustrative implementations of the present invention will be described with reference to the accompanying figures, in which:

FIG. 1 shows an exploded view of the components of an injection device according to a first embodiment of the invention.

FIG. 2 shows a longitudinal cross section of the exploded view shown in FIG. 1.

FIG. 3 shows a side view of an injection device according to a first embodiment of the invention with a screw plug in place of a needle hub (which may be provided separately).

FIG. 4 shows a longitudinal cross section of the device as illustrated in FIG. 3.

FIG. 5 shows a side view of an injection device according to a first embodiment of the invention with a needle hub installed, the needle hub also comprising a needle sheath with Pars plana measuring rim and protective cap substantially enshrouding the sheath and needle.

FIG. 6 shows a longitudinal cross-section of the device as illustrated in FIG. 5.

FIG. 7 shows longitudinal cross-sectional details of an actuation mechanism according to a first embodiment of the present invention, as well as its interaction with the remainder of the injection device.

FIG. 8 shows longitudinal cross-sectional details of the distal end of an injection device according to an embodiment of the invention, including an attachable/detachable needle hub (with sheath and covering cap) and its interaction with the remainder of the injection device.

FIGS. 9 to 11 show longitudinal cross-sectional details and workings of an injection device according to a first embodiment of the present invention during an injection sequence (FIG. 9: pressure just applied to proximal end of the injection device against a subject to be injected; FIG. 10: injection depth achieved and plunger released; and FIG. 11: pressure removed from proximal end and sheath returned and locked into needle-enshrouding position), with FIG. 9 showing a longitudinal cross section which is longitudinally turned 90° compared to FIGS. 10 and 11.

FIG. 12 shows close-up longitudinal cross-sectional details of the proximal end of the device as shown in FIG. 11, at a slightly different angle to show details.

FIG. 13 shows external details of the actuation mechanism housing according to a first embodiment of the invention.

FIG. 14 shows close-up longitudinal cross-sectional details of the proximal end of the device according to a first embodiment of the invention when primed for injection, showing details of the interaction between the main body and cap of the actuation mechanism housing as well as details of retention arms, restrainer and plunger when primed for injection.

FIG. 15 shows details of the distal end of a preferred, intravitreal injection embodiment according to the present invention.

FIGS. 16 and 17 show details of the interaction of retention arms, the restrainer, the plunger and the driving spring when primed for injection (FIG. 16) and once injection has been performed (and the plunger has been released).

FIGS. 18 to 20 show different close-up angles of retention arms according to the present invention viewed.

FIG. 21 shows an exploded view of the relevant components of an injection device according to a second embodiment of the invention, where those components include different components to the first embodiment illustrated in FIG. 1.

FIG. 22 shows a longitudinal cross section of the exploded view shown in FIG. 21.

FIGS. 23 to 25 show longitudinal cross-sectional views of an injection device according to a second embodiment of the invention: FIG. 23 shows such a view with a screw plug in place of a needle hub, which may be provided separately; FIG. 24 shows such a view with a needle hub installed, the needle hub also comprising a needle sheath with Pars plana measuring rim and pressure pad, and protective cap substantially enshrouding the sheath and needle; and FIG. 25 shows the device of FIG. 24 with the protective cap removed.

FIGS. 26 to 31 show longitudinal cross-sectional details and workings of an injection device according to a third embodiment of the present invention during an injection sequence. FIGS. 27 and 28: pressure just applied to proximal end of the injection device against a subject to be injected, with sleeve contacting restrainer, FIG. 28 being at a slightly different angle to show details of physical interactions; FIG. 29: injection depth achieved and plunger released; and FIGS. 30 and 31 pressure removed from proximal end and sheath returned and locked into needle-enshrouding position, FIG. 31 being at a slightly different angle to show details of physical interactions.

FIG. 32 shows an actuation mechanism housing according to a third embodiment of the invention.

FIG. 33 shows an actuation mechanism housing proximal dose adjustment cap according to a third embodiment of the invention.

FIG. 34 shows the proximal end of a plunger according to a third embodiment of the invention.

FIG. 35 shows an exploded view of the components of an injection device according to a fourth embodiment of the invention.

FIG. 36 shows a longitudinal cross section of the exploded view shown in FIG. 35.

FIG. 37 shows a longitudinal cross-sectional side view of an injection device according to a fourth embodiment of the invention with a screw plug in place of a needle hub, a longitudinal cross-section of the needle hub, showing its components, being illustrated underneath.

FIG. 38 shows a longitudinal cross-sectional side view of an injection device according to a fourth embodiment of the invention with a needle hub installed, the needle hub also comprising a needle sheath with Pars plana measuring eye-contacting surface and protective cap substantially enshrouding the sheath and needle. The cross-section shown is rotated along its longitudinal axis by 45° compared to FIG. 37 and showing the sides of the sleeve leaves, whereas FIG. 37 shows longitudinal ‘ribs’ extending internally beyond the sleeve leaves to engage the formulation chamber and hold it in place.

FIG. 39 shows another longitudinal cross-sectional side view of an injection device as illustrated in FIG. 38, but rotated along its longitudinal axis by 90° compared to FIG. 38.

FIG. 40 shows a lateral plan view of an injection device as illustrated in FIGS. 37-39, but without protective sheath cap or proximal cap.

FIGS. 41 to 43 show longitudinal cross-sectional details and workings of an injection device according to a fourth embodiment of the present invention during an injection sequence (FIG. 41: pressure applied to proximal end of the injection device against a subject to be injected, with injection depth achieved and dose about to be released; FIG. 42: dose delivered; and FIG. 43: pressure removed from proximal end and sheath returned and locked into needle-enshrouding position).

FIGS. 44 to 46 show details of the interaction of retention arm, the sleeve/restrainer, and the plunger during an injection sequence (FIG. 44: actuation mechanism primed for injection, with retention arm and sleeve/restrainer in drive member-restraining position; FIG. 45: actuation mechanism triggered, with retention arm and sleeve/restrainer in drive member-releasing position; FIG. 46: pressure removed from proximal end and sleeve returned to its distal position, with retention arm fully deflected outwards, locking the sleeve and sheath in distal, needle-enshrouding position).

FIG. 47 is a transparency showing the actuation housing and its interaction with the retention arm, the driving spring, and plunger.

FIG. 48 is a transparency showing the actuation housing and its interaction with the retention arm, the first spring, and plunger with sleeve/restrainer and sleeve spring in plunger-retaining position.

FIG. 49 is a transparency showing the actuation housing and its interaction with the retention arm, the driving spring, plunger, sleeve/restrainer and sleeve spring in plunger-retaining position, and proximal cap positioned on top of the actuation housing.

FIG. 50 shows details of a preferred sleeve/restrainer according to the invention.

FIG. 51 shows two views of details of an embodiment of a retention arm according to the invention.

FIGS. 52 to 56 show details of the interaction of retention arm, the plunger, the proximal cap and the actuation housing during a priming, ‘0’ calibration, dose setting and ‘dose delivered’ sequence (FIG. 52: injection device assembled, but without needle in place, with formulation chamber under stress; FIG. 53: formulation chamber seal perforated by needle, releasing some stress on formulation chamber and allowing proximal cap to come to rest on complementary surface of actuation housing; FIG. 54: proximal cap turned to release remaining stress on formulation chamber, and bringing retention arm retaining ledge and complementary plunger ridge into engagement, and thereby ‘zeroing’ the dosing system—proximal cap still resting on complementary surface of actuation housing; FIG. 55: proximal cap further rotated and lifting proximally off complementary surface of actuation housing thereby allowing dose setting as determined by distance proximal cap is lifted off complementary surface of actuation housing; FIG. 56: dose delivered, with proximal cap returning to rest on complementary surface of actuation housing).

FIGS. 57 and 58 show details of a preferred actuation housing according to the invention, FIG. 57 providing a side plan view and FIG. 58 providing a view into the inside of the actuation housing as viewed from the distal end.

FIG. 59 shows details of the insides of a preferred proximal cap according to the invention as viewed from the distal end.

FIG. 60 shows details of the interactions between the actuation housing, proximal cap and plunger.

FIG. 61 shows an exploded view of the components of an injection device according to a fifth embodiment of the invention.

FIG. 62 shows a longitudinal cross section of the exploded view shown in FIG. 61.

FIGS. 63 to 69 show longitudinal cross-sectional details and workings of an injection device according to a fifth embodiment of the present invention during priming (FIGS. 63 to 65), dose setting (FIG. 66) and injection (FIG. 67: pressure applied to distal end of the injection device against a subject to be injected, with injection depth achieved and dose being delivered; FIG. 68: dose delivered; and FIG. 69: pressure removed from proximal end and sheath returned and locked into needle-enshrouding position).

FIG. 70 shows a longitudinal cross-sectional side view of the four main components of an injection device according to a fifth embodiment of the invention comprising: an actuation mechanism 20d and components; formulation cartridge 240 and components; elongated housing 10d and components; and needle hub 150d and components.

FIG. 71 shows an exploded view of actuation mechanism 20d (as shown in FIG. 70) and components.

FIGS. 72 to 76 show details of restrainer 130d, retention toggle 120d, plunger (plunger) 80d according to a fifth embodiment of the invention and their interaction/connections.

FIGS. 77 to 79 show details of the interaction of retention toggle 120d, sleeve leaves 76d, restrainer 130d, and plunger 80d during an injection sequence (FIG. 77: actuation mechanism primed for injection, with retention toggle, sleeve and restrainer in plunger-retaining position; FIG. 78: actuation mechanism about to be triggered, with sleeve and restrainer only just in drive member-releasing position; FIG. 79: actuation mechanism triggered, with toggle rotated about axis 121d and sleeve and restrainer in drive member-release position.

FIGS. 80 to 82 show external and internal details of an actuation housing 110d according to a fifth embodiment of the present invention.

FIGS. 83 and 84 show external and internal details of a proximal cap 290d according to a fifth embodiment of the present invention.

FIG. 85 shows an exploded view of elongated housing 10d (as shown in FIG. 70) and components.

FIGS. 86 and 87 show external and internal details of an elongated housing 10d according to a fifth embodiment of the present invention.

FIGS. 88 and 89 show external and internal details of a sleeve 70d according to a fifth embodiment of the present invention.

FIGS. 90 and 91 show external and internal details of a cartridge centering/spring retaining plug 440 according to a fifth embodiment of the present invention.

FIG. 92 shows a longitudinal cross-sectional side view of an elongated housing 10d according to a fifth embodiment of the present invention (as shown in FIG. 70), showing its components and their relative positioning.

FIGS. 93 to 95 show details of a needle hub according to another specific embodiment of the present invention.

FIGS. 96 and 97 show details of another specific embodiment of a dose-setting mechanism according to the present invention: FIG. 96A—external details; FIG. 96B longitudinal cross-section showing internal details; FIG. 97 partial cut-away section showing further details.

FIG. 98 show details of an alternative dose-setting mechanism according to an embodiment of the present invention.

FIGS. 99 and 100 shows details of a self-setting retention arm mechanism according to an embodiment of the present invention.

FIGS. 101 and 102 show details of a camming retention arm (not requiring a biasing spring for operation) and its operation.

FIGS. 103 to 107 show details of alternative plunger driving mechanisms for use in various embodiments according to the present invention.

FIG. 108 show details of the use of a multi-use injection device with replacement of elongated housing (complete with needle assembly and formulation chamber) according to an embodiment of the present invention.

### DEFINITIONS

As used herein, the term “adaxial” means towards or facing an axis. The term “abaxial” means away from or facing away from an axis.

As used herein, the term “distal” means the end or direction of injection. The term “proximal” means the end or direction toward the rear of the injector or toward the rear of the injection member and/or injector and/or injection member.

As used herein, the term “engage” means that two or more items interact such that one item acts on one or more other items with which it ‘engages’ and the items that ‘engage’ may connect with one another or may simply make contact with one another. Corresponding terms such as “engages”, “engaged” and “engagement” are intended to have corresponding meanings.

### DETAILED DESCRIPTION

The present invention will now be described by way of reference to various specific embodiments exemplifying the invention which is, however, not to be so limited, as various modifications may be made to the specific embodiments described herein without departing from the inventive concept illustrated by them. It will in particular be understood that the various embodiments are intended to be illustrative and not limitative. In particular, it will be understood that the disclosure encompasses the substitution of equivalent mechanical structures, for example kinematic inversions, alternative bias means, and variations in geometry and materials. It will also be appreciated that devices intended for different volumes or for injectable materials of different viscosity or consistency may require corresponding adaptations to the device structures and dimensions.

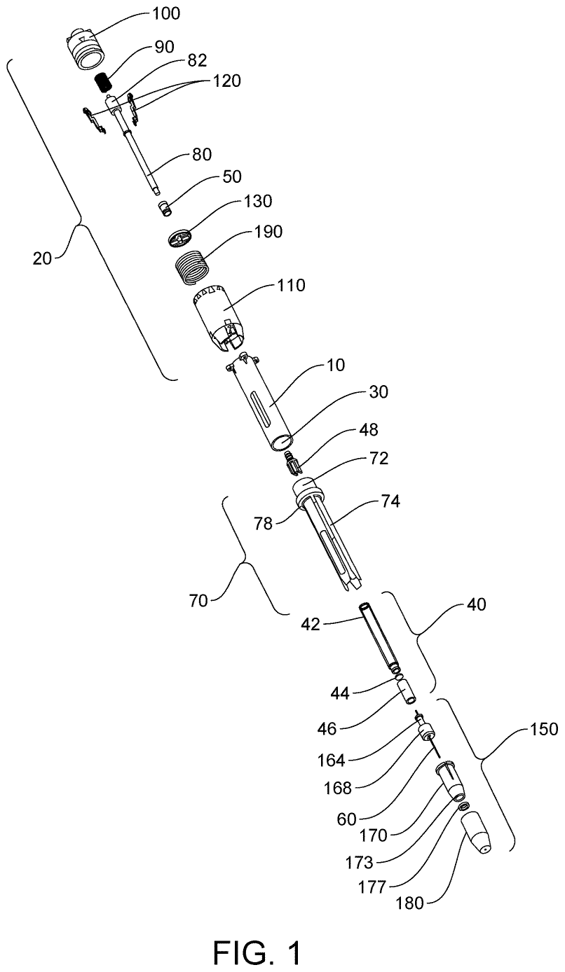

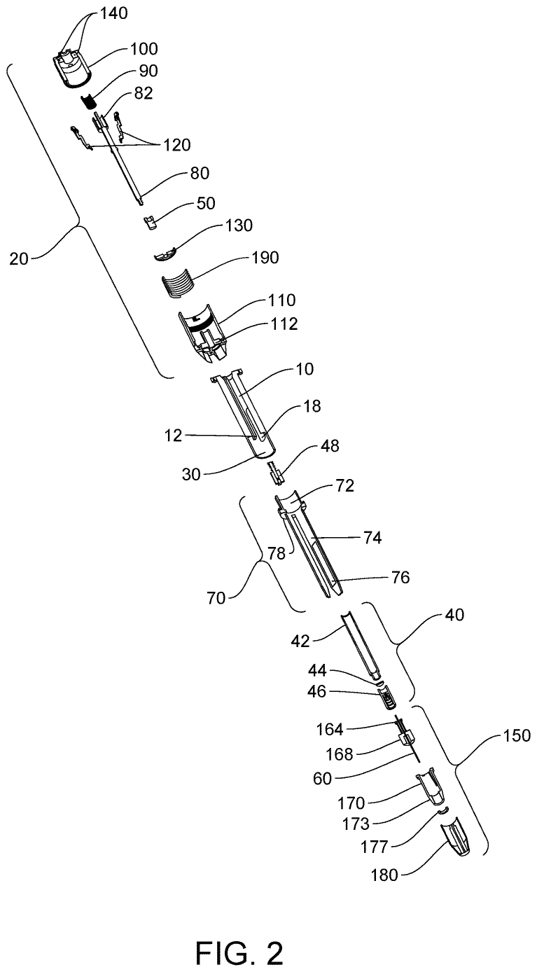

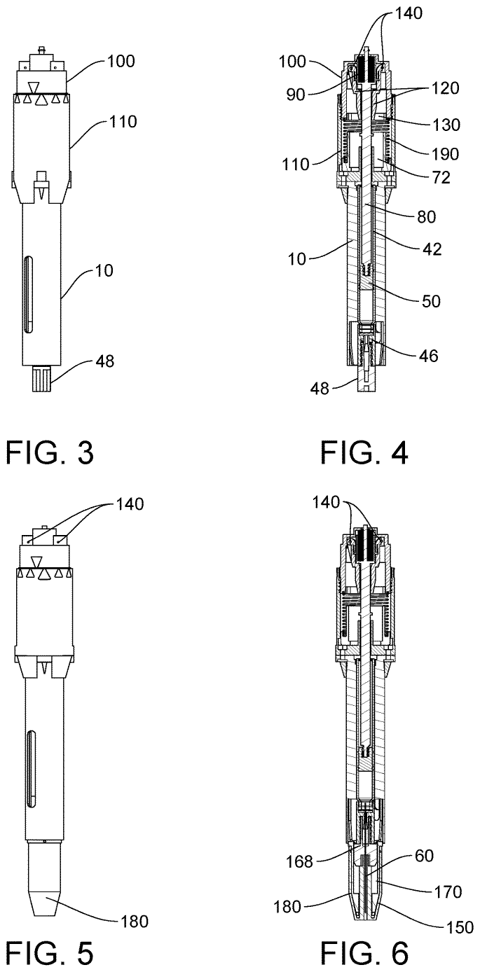

Having reference to FIGS. 1 to 6, a first embodiment of an injection device according to the present invention comprises a substantially hollow elongated housing 10 having proximal and distal ends, with an actuation mechanism 20 engaged at its proximal end, the actuation mechanism 20 comprising an actuation housing defined by proximal cap 100 and distal body portion 110. The elongated housing 10 comprises internally extending longitudinal ribs 12 which define an inner lumen 30 adapted to receive and secure a formulation chamber 40 containing, or adapted to contain an active agent for injection into a subject and the formulation chamber also comprises corresponding proximal and distal ends. Stopper 50 is provided at least at the proximal portion of chamber 40 for retaining active agent therein. The distal end includes, or is adapted to be in fluid communication with, a needle 60. The elongated housing also includes a sleeve 70 with leaves 74 which are positioned between the ribs, so that sleeve 70 is at least proximally slidable with respect to elongated housing 10.

In certain embodiments proximal cap 100 may engage with body portion 110 via complementary screw threads 105 and 118 on proximal cap 110 and body portion 110 respectively, allowing for adjustment of driving member or plunger 80 (and stopper 50) relative to formulation barrel 42, and therefore for priming of needle 60 and adjustment of dosage to be delivered.

Elongated housing 10, actuation housing components 100 and 110 and sleeve 70 can comprise any suitable material as used in the art, including suitable polymeric materials, metals and/or metal alloys. In certain aspects, elongated housing 10, actuation housing components 100 and 110 and sleeve 70 may be constructed of a polymeric material known to one having ordinary skill in the art, including but not limited to polycarbonate (‘PC’), acrylonitrile, butadiene, and styrene (‘ABS’) blends, polyethylene, polystyrene, or polyvinyl chloride. Such material may be manufactured into the aforementioned components by suitable means known to one having skill in the art, such as moulding.

Each of the elongated housing 10, sleeve 70, proximal cap 100, and body portion 110 may conveniently have at least internal profiles that are hollow cylindrical (optionally with two or more stepped inner diameters, and having additional features as described further below), especially elongated housing 10 and sleeve 70, given the cylindrical profile of commercially available formulation chambers. However, external profiles are not necessarily so limited, and may include shaping that makes the injection device more ergonomic or easier to manufacture or package, and even the inner profiles may be other than cylindrical, although this may come with manufacturing, ease of assembly and/or functionality complications.

In the specific embodiment illustrated in FIGS. 1 to 14 sleeve 70 comprises a hollow cylindrical proximal body 72 which is housed within the actuation housing defined by proximal cap 100 and distal body portion 110, with sleeve leaves 74 extending distally from body 72, through corresponding slots in baseplate 112 of housing body portion 110 and into lumen 30 of elongated housing 10. One or more sleeve leaves 74 may be omitted or comprise a window 76 which omission or window may coincide with a window 18 in elongated housing 10 such that the contents of formulation chamber 40 (if installed) can be viewed. In an alternate embodiment, the body of the elongated housing or other components may be composed of materials which are transparent or translucent, thus facilitating viewing of the contents of the formulation chamber or other internal components.

Elongated housing 10 and actuation mechanism 20 may engage with one another by any suitable means, such as those known in the art, which will ensure that in use they remain in a fixed positional relationship relative to one another. For example, the mutually compatible surfaces may be welded, crimped, snapped, screwed or glued together.

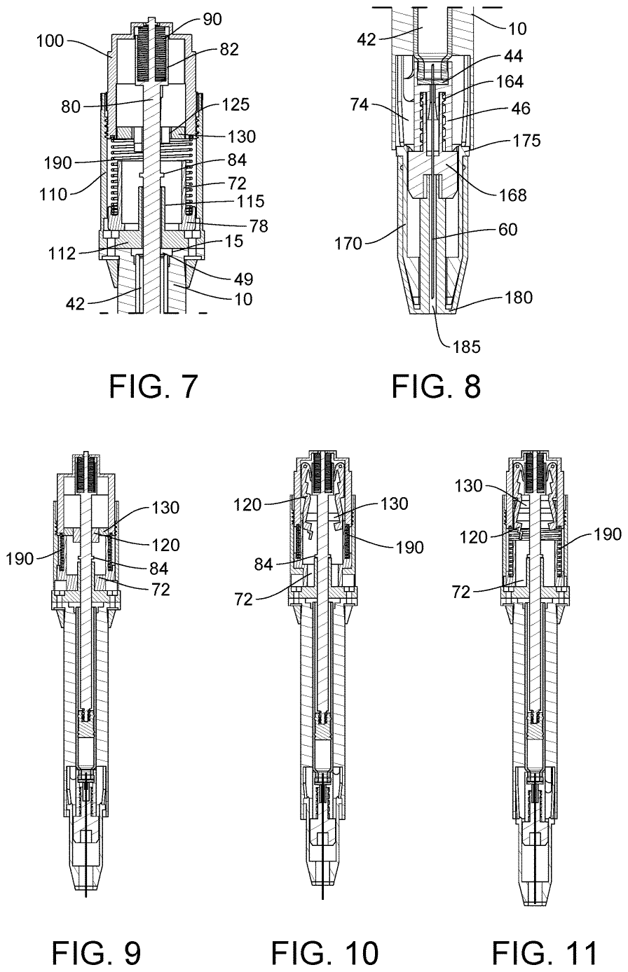

According to an embodiment, actuation mechanism 20 may comprise a driving member or plunger 80 the distal end of which engages with, or is adapted to engage with stopper 50 of the formulation chamber. As well as interlock, overlap, screw into or onto, amongst others, ‘engage’ and corresponding terms may include mere pushing against, such that plunger 80 may be adapted to simply come up against and push stopper 50. At its proximal end plunger 80 engages with actuation mechanism housing cap 110, with at least one driving spring 90 positioned between cap 100 and plunger 80. Plunger 80 may comprise any suitable means for holding driving spring 90 between plunger 80 and cap 100, such as spring retaining cup or ridge 82 shown in FIGS. 1, 2, 4 and 6 (as well as FIGS. 7, 9-12, 16 and 17) formed close to the proximal end of plunger 80.

Having reference to FIGS. 4, 6, 7, 9-12, 14, 16 and 17, when an injection device according to the present invention is put together, with formulation chamber 40 in place and plunger 80 engaged with stopper 50, and the device is primed for injection, driving spring 90 is held under stress with plunger 80 held in position by retention arm 120. To best control retention arm release force (i.e., force that needs to be applied against a surface, such as the eye, in order to actuate the injection device), a retention arm 120 is provided with outwards bias (i.e., away from plunger 80), with retention arm being held in plunger engaging position against the bias by a restrainer 130 which is at least proximally slidable, restrainer 130 being in restraining position when distal (FIGS. 4, 6, 7, 9, 14 and 16) and in releasing position when proximal (FIGS. 10, 11, 12 and 17). In use, restrainer 130 is driven proximally by sleeve 70 when sleeve 70 is slid proximally relative to elongated housing 10 by applying distal pressure on the injection device against a surface to be injected, thereby releasing retention arm 120 to deflect away from plunger 80, allowing driving spring 90 to drive plunger 80 and stopper 50 distally.

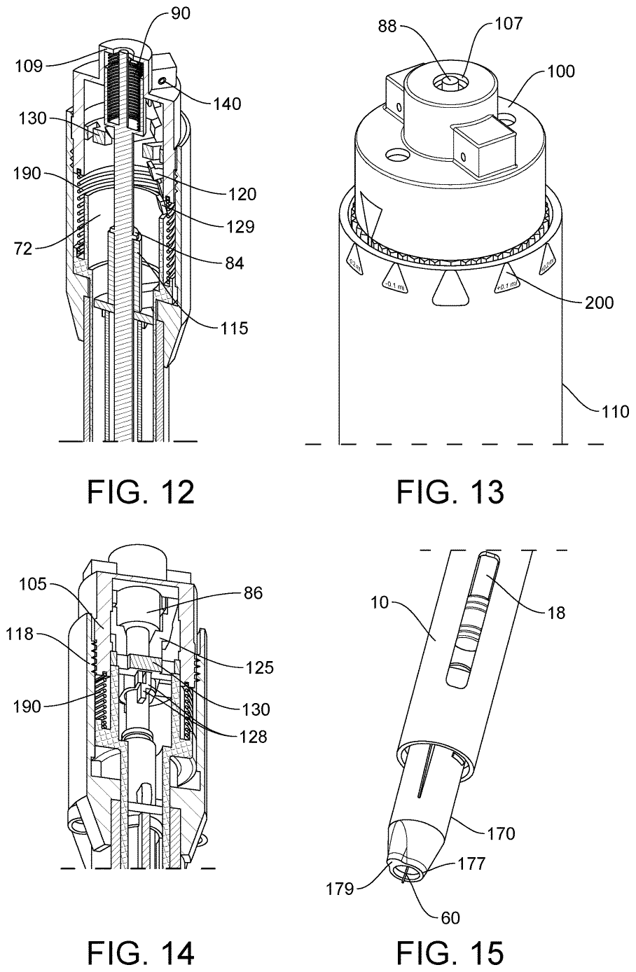

Having reference to FIGS. 12, 14, 16-20, 41-47 and 51, preferred forms of retention arm 120, 120c and restrainer 130 and 130c are illustrated although once given the benefit of this disclosure other suitable means/configurations will be readily apparent to those skilled in this area, and the invention is not to be seen as being limited to the particular preferred forms of retention arm or toggle and restrainer illustrated. Alternative retention arms or toggles and complementary restrainers may, for example, comprise different configurations and/or single or three or more retention arms/toggles held against their bias by similar restrainers such as restrainer 130 as illustrated, or one or more different means, other than a shaped disc as illustrated.

Having reference to the retention arm 120 as illustrated in FIGS. 12, 14, and 16-20, each comprises an extended ‘arm’ with a pivot/fulcrum point 121 at the proximal end (like a shoulder) adapted to engage a pin or axle 140 (shoulder joint) provided at the proximal end of proximal cap 100. Alternatively, pin(s) or axle(s) 140 may be formed separately or even form part of retention arm 120 and engage with proximal cap 100, or corresponding part of the actuation mechanism housing via corresponding joints or fulcrum points. In one embodiment, in the ‘armpit’ portion of retention arm 120, adaxially situated is a spring arm 123 (which may be formed integrally with retention arm 120 or separately, and may be made of a stiff, elastomeric or springy material, which may be a the same or a different material than the rest of retention arm 120) with corresponding recess 124. In an alternative embodiment a spring or spring arm may be attached to the side of plunger 80, or even be an integral part of plunger 80, and push against retention arm 120, in certain embodiments against a surface of retention arm 120 corresponding with recess 124. Moving distally along the ‘arm’ of retention arm 120 is a step in or axially oriented ‘elbow’ forming an inwardly directed ledge 125 which, when in plunger-engaging and retaining position, is substantially perpendicular to the axis of plunger 80 and adapted to engage an opposed ridge or ledge 86 formed on plunger 80, and which may conveniently be the lower, outer edge of spring retaining cup 82. Retention arm 120 extends distally beyond inwardly directed ledge 125 (thus forming the equivalent of a ‘forearm’ in the ‘arm’ analogy), ending with a ‘hand’ comprising a concave flange 127 adapted to rest against the shaft of plunger 80, concave flange 127 having further flanges or tabs (retention tabs) 128 extending outwardly either side of it, one of retention tabs 128 (the right hand side one, looking from the adaxial side, in this example) having a further distally extending flange/tab 129 (‘sheath locking tab’). Retention arm 120 may be made of any suitable material as used in the art, including suitable polymeric materials, metals and/or metal alloys and, as mentioned above, may be made of a combination of any such materials. Where spring arm 123 is an integral part of the whole retention arm 120, retention arm 120 may be made of a suitable firm, elastomeric polymeric material such as polycarbonate, polyoxymethylene, nylon and the like. Triggering pressure (i.e., the amount of proximally directed force that needs to be applied by sleeve 70 against restrainer 130, and therefore distal force that needs to be applied against a surface to be injected) using such a retention arm is proportional to the ratio a:b between the distance “a” between pivot point 121 and the proximal surface of tab 128 and the adaxial extension distance “b” of the edge of ledge 125 compared to pivot point 121, so for a given bias strength provided by spring arm 123, the longer “a” is, or the smaller “b” is, the smaller the force required to trigger the device, allowing for less discomfort to a subject being injected. Therefore, importantly for certain applications such as intraocular injections, the triggering pressure can be adjusted through adjustment of the ratio a:b in this particular embodiment, and through adjustment of equivalent mechanical advantage ratios for other injection device embodiments described below.

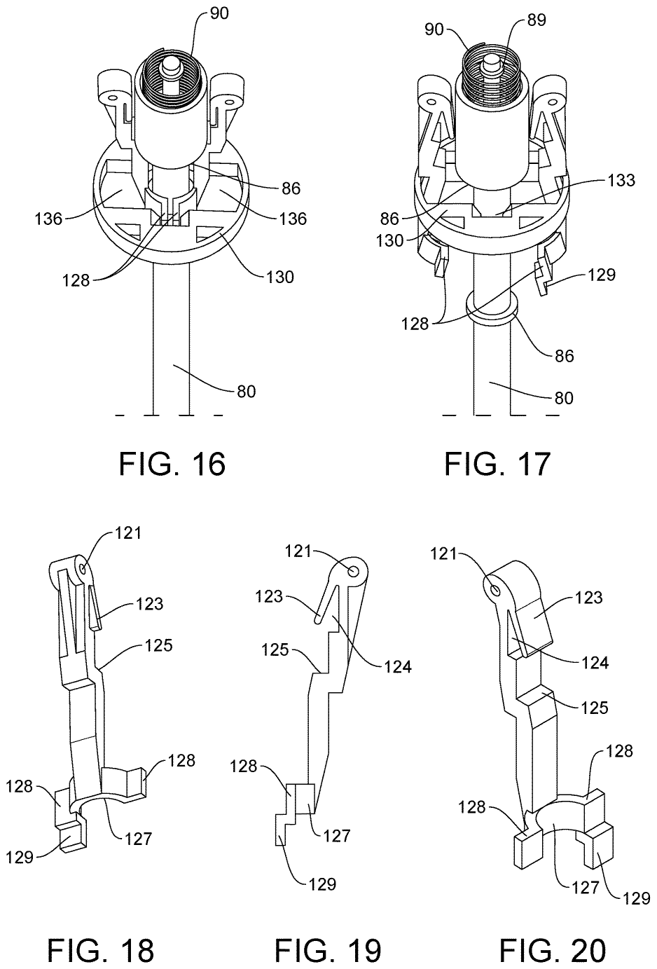

Having reference to the restrainer 130 as illustrated, this is provided as a flat disc which may be any suitable thickness, but typically from about 2 mm to about 50 mm, typically from about 5 mm to about 20 mm, and may be made of any suitable material as used in the art, including suitable polymeric materials, metals and/or metal alloys. Having reference to FIGS. 16 and 17, in one embodiment restrainer 130 may have suitable spaces cut into it: i) opposed retaining channels 133 to hold the opposed pairs of retention tabs 128 to hold the pair of retention arm 120 against plunger 80 and against bias generated by compression of spring arm 123 into recess 124 when in a retaining position; ii) releasing channel 136 to provide space for retention arm to deflect away from plunger 80 when restrainer 130 is slid proximally to release retention tabs 128; and iii) space created at the cross-section of opposed retaining channels 133 and releasing channels 136 to allow restrainer 130 to fit over plunger 80 and move freely along at least that part of plunger 80 engaged by the lower portion of retention arm 120 (below ledge 125).

The embodiment illustrated in FIGS. 1-12 is adapted to utilise a pre-filled needleless formulation chamber 40, which may optionally be vacuum or voidless filled, and to have a separately provided needle hub 150 attached, via any suitable mechanism known in the art, such as tight fit, snap, Luer lock, bayonet or screw mechanisms. However, formulation chambers, pre-filled or not, with integral or otherwise attached needles may also be employed, as described below. In the embodiment illustrated in FIGS. 1-12, and with particular reference to FIGS. 1-4, pre-filled formulation chamber 40 as may, for example, be provided separately or form an integral part of the injection device as provided, comprises a cylindrical barrel 42 (which may be made of glass or a suitable polymeric material, optionally silanized or siliconized) with seal 44 and barrel connector 46 (which may be formed integrally with barrel 42, if made of polymeric material, or may be formed separately and be attached to the distal end of barrel 42, such as by gluing, snapping, crimping or other suitable means), as well as a stopper 50, with formulation comprising active agent being contained within barrel 43 between seal 46 and stopper 50. Pre-filled needleless formulation chambers such as illustrated in FIGS. 1-12 also have a barrel flange at the proximal end of barrel 42 projecting axially outwards and typically used for providing purchase for a forefinger and middle finger for holding the syringe while applying pressure on the proximal end of a plunger, to perform injection. However, in the embodiment illustrated in FIGS. 1-12, and especially as illustrated in FIGS. 7-12, according to one embodiment formulation chamber 40 may be retained in the injection device in the lumen formed by elongated housing 10, with barrel flange 49 at the proximal end of barrel 42 sitting in a recess 15 formed in the proximal end of elongated housing 10 and which, together with baseplate 112 of actuation housing distal body portion 110, secures formulation chamber 40 in position within the injection device. A close fit of formulation chamber 40 within elongated housing 10 provides further rigidity to the retention of formulation chamber 40 within the injection device. Typically a pre-filled formulation chamber 40 will be provided a syringe cap 48 screwed into barrel connector 46 to ensure sterility and cleanliness within the barrel connector until syringe cap 48 is removed prior to attachment of needle hub 150. In other embodiments plunger 80 may assist in holding formulation chamber 40 in place by applying pressure against stopper 50.

In the embodiment described immediately above, and with reference to FIGS. 1, 2 and 8, a needle hub 150 may comprise a needle 60 extending from both sides of an overmoulded body 160 having a proximal connector end 164 adapted to engage and connect with barrel connector 46 and having a shorter end of needle 60 extending proximally therefrom sufficiently to pierce seal 44 when hub 150 is fully connected with barrel connector 46, and a main substantially cylindrical body 168 adapted to fit closely, but slidably within sheath 170 which has a proximal end 175 adapted to clip around body 168 thereby retaining body 168 within sheath 170, but with connector end 164 protruding proximally from sheath 170. In an alternative embodiment needle hub 150 is made in two parts, each part with an over moulded needle (a proximal needle to pierce seal 44 or 246, and a distal needle for performing the injection) which are then joined together (needles pointing in opposing directions) with a filter between them. This would be so that any particles that may be generated in piercing the prefilled cartridge or prefilled syringe, or even any microparticulate matter present in the formulation are filtered out. Proximal end 175 is also adapted to engage sleeve 70 when hub 150 is fully connected with barrel connector 46. When not in use, hub 150 (whether attached to connector 46 or not) may comprise a cap 180 ideally adapted to clip onto sheath 170. In certain embodiments cap 180 may comprise a guide 185 for securing and protecting needle 60 within the needle hub. Alternatively, or in addition, the distal end or surface 173 of sheath 170 may comprise a guide for securing and protecting needle 60 within the needle hub. Having reference to at least FIGS. 1, 2 and 15, distal surface 173 of sheath 170 may act as the surface for contacting a subject, or body part thereof, for injection. Such a contacting surface of said sheath may, in further embodiments, comprise a pad 177 made of softer and/or spongier and/or adherent material than sheath 170, such as silicone or rubber or an absorbent material. In further embodiments such distal surface 173 of sheath 170 may be provided with features such as protrusions, ridges and/or other textural elements. In yet further embodiments, the contacting surface of the distal surface of sheath may include a combination of the aforementioned features or materials. Such materials and features may assist in holding the distal surface in position (for example in the correct injection position on the eye surface for intraocular injection) during the injection procedure, and may further assist the clinician in controlling or retaining the position of the eye in a desirable position during the injection. Although not shown, needle hub 150 may comprise a biasing means, such as a spring, within sheath 170, which urges sheath 170 distally with respect to body 168 and therefore needle 60, to keep needle 60 sheathed when not in use. Sheath 170 may be substantially hollow cylindrical for all or most of its length, although other shapes may be contemplated. In certain embodiments sheath 170 may have a frustoconical distal end. Where sheath 170 and corresponding cap 180 are substantially cylindrical in shape, sheath 170 or cap 180 may comprise means to avoid axial rotation of cap 180 relative to sheath 170, to allow use of rotational pressure on cap 180 to engage needle hub 150 with barrel connector 46. Such means may, for example, comprise complementary longitudinal (i.e., distal-proximal extending) ridges and grooves, or introduction of some form of asymmetry in sheath 170 and cap 180 sufficient to stop axial rotation of cap 180 relative to sheath 170. Further alternative means may, for example, comprise: a pin or stiff material slip that passes through corresponding holes formed in sheath 170 and cap 180; or ribs extending from hub body 168 through corresponding slits in sheath 170 and engaging with corresponding slots in cap 180. In certain aspects cap 180 is adapted to holding the remaining components of needle hub 150 rigidly in place relative to each other to enable handling and attachment of the hub to barrel connector 46, such adaptations including, for example, clippable flanges, interacting complementary fins and slots or grooves and ridges, biasing means, and the like. In certain aspects cap 180 may be closed and have a distal internal void large enough to take a priming volume or dose adjust volume of formulation and the void may also have an absorbent pad to absorb that volume.

Instead of a needleless formulation chamber 40 as per the embodiment illustrated in FIGS. 1-12, a formulation chamber with needle (integral with barrel or not) may also be used. In such an embodiment there would be no need for a needle hub. However, in certain embodiments a sheath may be provided as an extension of sleeve 70, and this may comprise a cap covering the end of the injection device when not in use.

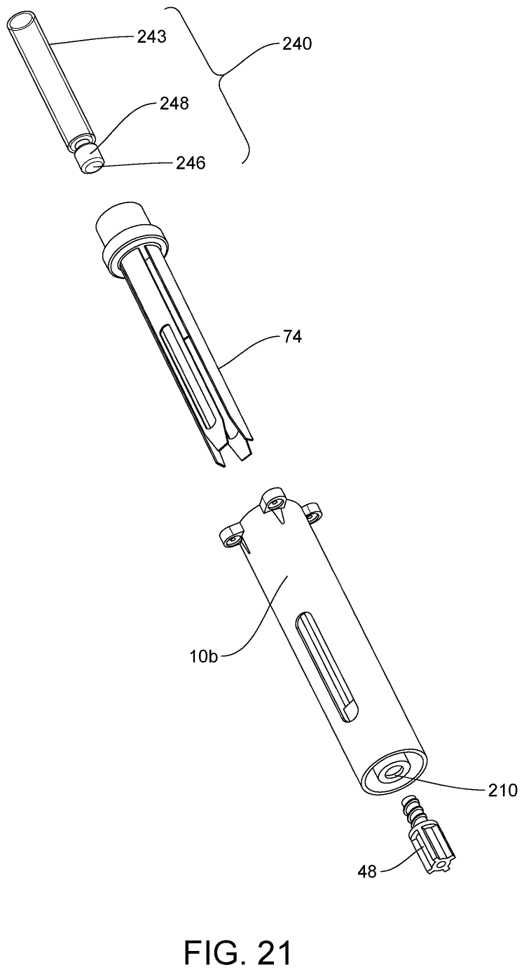

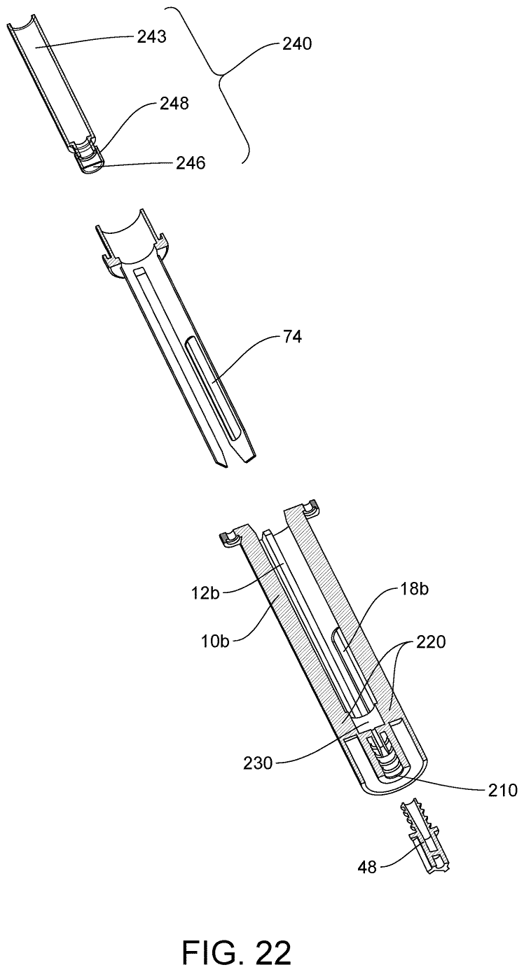

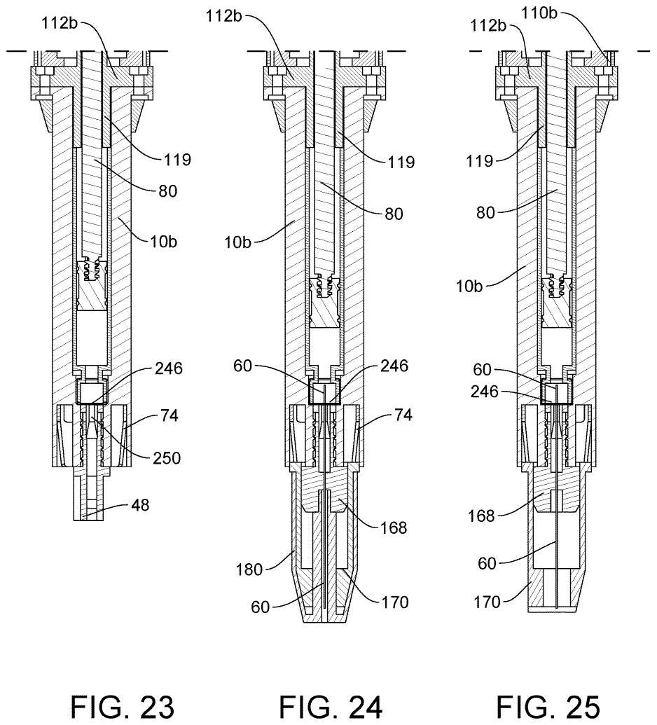

Readily available formulation chambers also include replaceable or disposable cartridges, which typically are not provided with barrel connectors but, instead, comprise a barrel with a seal at the distal end that is held in position by a crimped ring, or similar, a stopper towards the proximal end, and no barrel flange. Having reference to FIGS. 21 to 25, a second embodiment of injection devices according to the present invention is provided which encompasses the use of such replaceable or disposable cartridges, which involves a change in the configuration of elongated housing 10, and FIGS. 21 to 25 provide the details of those changes. In particular, because replaceable or disposable cartridges do not have a barrel connector, according to the present embodiment a needle hub connector 210 is provided as an internally threaded collar supported by radially intruding extensions 220 of elongated housing 10b. Radially intruding extensions 220 provide slots between them, corresponding with equivalent slots in base plate 112 of body portion 110, for sleeve leaves 74 to move within, allowing for the use of the same sleeve 70 as described above. The proximal end of needle hub connector 210 provides a recessed seat 230 for accommodating the distal end of replaceable or disposable cartridge 240 (comprising barrel 243 which may be made of glass, optionally silanized, or a suitable polymeric material, optionally siliconized, seal 246, typically but not necessarily made of synthetic rubber, such as butyl rubber, and crimped ring 248, typically but not necessarily made of aluminium, which holds seal 246 in place against the distal rim of barrel 243). Between recessed seat 230 and hub connector 210 is a channel 250 allowing for the proximal end of needle 60 to pass through from connector hub 210, through seal 246 and into the lumen of barrel 243. In one embodiment, in order to secure replaceable or disposable cartridge 240 into position within elongated housing 10b, the body portion of the actuation housing may be modified to provide body portion 110b having a modified base plate 112b comprising a distally extending collar 119 which surrounds plunger 80 and extends into the lumen within elongated housing 10b and sleeve 70, said collar extending into said lumen the correct distance to ensure that replaceable or disposable cartridge 240 is secured in said lumen such that it is substantially immobilised therein. Having reference to FIGS. 23 to 25, needle hub 150, syringe cap 48, as well as the remainder of actuation mechanism 20 (other than body portion 110b) can be the same as, or similar to those as already described above, and as shown in FIG. 22, elongated housing 10b may include the option of a window 18b which coincides with a window 76 in sleeve leaf 74, such that the contents of replaceable or disposable cartridge 240 (if installed) can be viewed.

Having described the main components of certain injection devices according to the present invention, its mechanics during use will now be described.

Firstly, and with reference to FIGS. 1 to 20, the device may be provided as a single-use, pre-packaged sterile device ready for injection, or it may be provided without formulation chamber, either embodiment optionally without needle hub 150 (provided separately in a sterilised packaged form). If a formulation chamber 40 is provided separately, ideally pre-filled, actuation mechanism 20 with sleeve 70 is separated from elongated housing 10 and formulation chamber 40 is inserted into elongated housing 10 with barrel flange 49 seated into recess 15. Sleeve leaves 74 are then positioned around barrel 42 and elongated housing 10 and actuation mechanism 20 are brought together and fixed in position, at the same time engaging the distal end of plunger 80 with stopper 50. A needle hub 150 is then connected to barrel connector 46 (if not already connected, or if a formulation chamber with integral needle hub is not being used), with plug 48 being removed first.

If the formulation chamber is vacuum filled, this assembly will result in positive pressure within formulation chamber 40 and, once the proximal end of needle 60 pierces seal 44 the positive pressure within formulation chamber 40 will result in expulsion of formulation from barrel 42 into needle 60, resulting in a self-priming system.

Once the injector is assembled, restrainer 130 will be in its distal, restraining position, with opposed pairs of retention tabs 128 of retention arm 120 held substantially together in retaining channels 133 (against abaxial bias resulting from compression of spring arms 123 into recesses 124), and substantially against plunger 80, with inwardly directed ledge 125 engaging with opposed ridge/ledge 86 of plunger 80, thereby holding the plunger in ‘primed’ position against bias provided by driving spring 90.

If the system is not self-priming, according to one embodiment the system may be primed by adjusting the position of proximal cap 100 relative to body portion 110 by screwing proximal cap 100 into body portion 110 sufficiently to prime needle 60. In certain embodiments the dosage to be delivered during injection may also be controlled by adjusting position of proximal cap 100 relative to body portion 110 and therefore the travel distance of plunger 80 during injection.

It will be appreciated that operation of both the self-priming mechanism, or the dosage adjustment by operation of cap 100, cause a quantity of the supplied dosage in the formulation chamber to be expelled from the injection device.

Once the system is primed, and dosage set if required, the distal surface of sheath 170 (or pad 170 if provided) is placed against the surface to which injection is to be made and pressure applied in a distal direction. This pressure results in sheath 170 sliding proximally into elongated housing 10 and against the distal ends of sleeve leaves 74 and corresponding proximal sliding of sleeve 70 up into the actuation housing defined by proximal cap 100 and distal body portion 110 and against bias provided by at least one sleeve spring 190 between the actuation housing and spring-engaging ridge or cup 78 on the outer circumferential edge of sleeve body 72. As sleeve 70 moves proximally into the actuation housing its proximal surface engages the distal surface of restrainer 130, pushing restrainer 130 proximally. Once restrainer 130 has moved the required distance proximally (as a result of needle 60 reaching the desired injection depth) retention tabs 128 are no longer held together and against plunger 80 in channels 133 and, as a result, the bias provided by spring arms 123 deflects the ‘arms’ of retention arm 120 away from plunger 80 and into the space provided by releasing channel 136 (with the abaxial deflection of the distal ends of retention arm 120 being limited by the inner wall of sleeve 70), breaking the engagement of ledge 125 and ridge/ledge 86 and releasing plunger 80 which, under pressure from first spring 90, is driven distally, resulting in formulation being expelled from the distal end of needle 60.

In certain embodiments plunger 80 has a ridge 84 and base plate 112 of body portion 110 has a proximally extending collar surrounding a portion of the length of plunger 80 and providing an upper surface 115 and together, ridge 84 and surface 115 define the end travel of plunger 80 during injection, when ridge 84 reaches, and is stopped by surface 115. This, especially together with adjustment of proximal cap 100 relative to body portion 110, allows for a defined travel distance for plunger 80, and therefore stopper 50, during injection and therefore for a delivery of a defined dosage. In certain embodiments proximal cap 100 and/or body portion 110 may have dosage markings 200 calibrated for particular formulation chambers.

Once injection has been performed, distally applied pressure is eased or removed and, as a result, sleeve spring 190, applying pressure on ridge or cup 78 of sleeve 70, which is no longer opposed, urges sleeve 70 distally, which in turn results in sheath 170 being urged distally to once again cover needle 60. Also, as a result of sleeve 70 being urged distally, its inner wall no longer limits the further abaxial travel of retention arm 120, which then deflects further abaxially towards the walls of body portion 110 of the actuation mechanism housing (and radially outward of pivot point 121), with sheath locking tabs 129 then obstructing potential proximal travel of sleeve 70 and therefore of sheath 170, thereby locking sheath 170 into a needle covering position, making the device safe (FIGS. 11 and 12; see also FIGS. 30 and 31). The proximal end of the sleeve may be adapted to better engage the sheath locking tabs/surfaces of the retention arm or toggle, and therefore more strongly lock the sleeve and thereby the sheath in a needle-covering position. Such adaptations may, for example, include notches in the sleeve, or even the proximal edge of the sleeve having flexible fingers, for engaging the sheath locking tabs or surfaces of the retention arm or toggle, although several other such means will be readily apparent to those skilled in this art.

With reference to especially FIGS. 12, 13, 16 and 17, a convenient adaptation of the injection devices according to the invention may be provided which allows sensing of the injection mechanism having been triggered. In particular, plunger 80 may comprise an extension 88 which extends proximally through a collar 107 at the proximal end of actuation housing cap 100, and said extension moves/recedes into cap 100 when the injection device is actuated, providing a signal that the device has in fact been actuated. Alternatively, extension 88 may comprise a ridge 89, and collar 107 may comprise a thin flap, flaps or ridge 109 of suitably stiff, elastomeric or springy material (and may be formed integrally with cap 100 or separately, and may be made of the same or different material to cap 100) extending adaxially to within the diameter of the abaxial extension of ridge 89 and, when injection is performed, with plunger 80 being driven distally, ridge 89 is driven past flap, flaps or ridge 109 resulting in deflection of flap, flaps or ridge 109 in such a manner as to result in a noise, such as a ‘click’ signalling that the injection mechanism has been triggered.

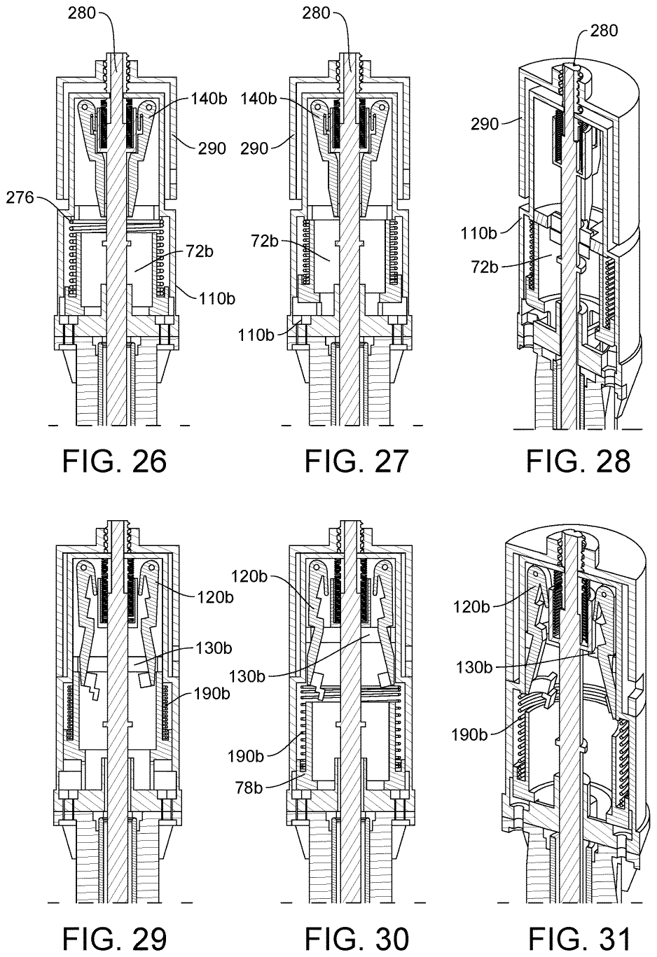

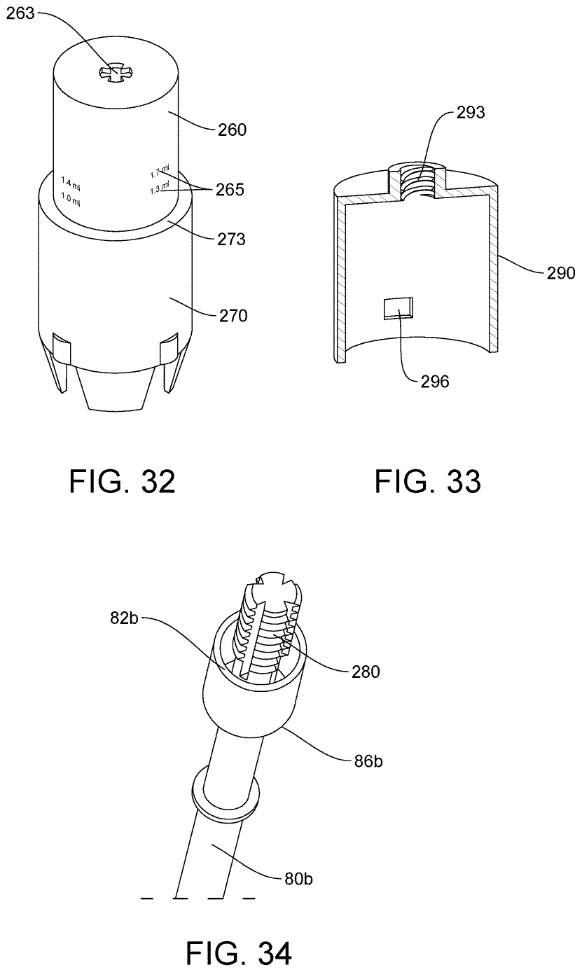

In a third embodiment of injection devices according to the present invention, an alternative configuration of the actuation mechanism to control the dose of formulation dispensed by the device during injection is provided. Referring to FIGS. 26 to 34, compared to the previously described embodiments, the actuation mechanism housing is modified such that the actuation mechanism housing is defined by body portion 110b, which comprises a proximal portion 260 and a distal portion 270, wherein proximal portion 260 has a narrower diameter and inner bore than distal portion 270 and houses retention arm 120b (which may be the same as retention arm 120 as previously described, and connected to the proximal end of proximal portion 260 via pins or axles 140b, substantially as previously described) and restrainer 130b is also slidably retained within proximal portion 260. The inner bore of proximal portion 160 is slightly greater than the outer diameter of hollow cylindrical proximal body 72b of sleeve 70b such that hollow cylindrical proximal body 72b may be slidably received by proximal portion 260 during the injection procedure and drive retaining member 130b proximally. The interface of proximal portion 260 and distal portion 270 forms an outer ledge 273 and an inner ledge 276 that, with spring retaining ridge/cup 78b holds sleeve spring 190b in position in distal portion 270. As well as modification of the actuation mechanism housing, plunger 80b is also modified compared to plunger 80 previously described, having a threaded extension 280 that engages with a corresponding thread 293 in cap 290 which in turn fits slidably over proximal portion 260 of body portion 110b. Plunger 80b is otherwise substantially the same as plunger 80 previously described, including spring retaining cup 82b and ridge/ledge 86b. However, a ridge such as ridge 84 no longer has a role, as the travel distance of plunger 80b, and therefore the amount of formulation dispensed during injection is now determined by the relationship between plunger 88b, cap 290 and proximal portion 260 of body portion 110b: once an injection device according to this embodiment is assembled, a desired dosage may be dialed up by screwing cap 290 proximally until the desired dosage marking 265 shows through window 296. This lifts cap 290 proximally off ledge 273, with the distance between the lower surface of cap 290 and ledge 273 defining the travel distance of plunger 80b, and therefore volume of formulation dispensed during injection. When the actuation mechanism is triggered, as described earlier, the lower surface of cap 290 is driven to surface 275, providing a convenient indicator that the device has been successfully triggered.

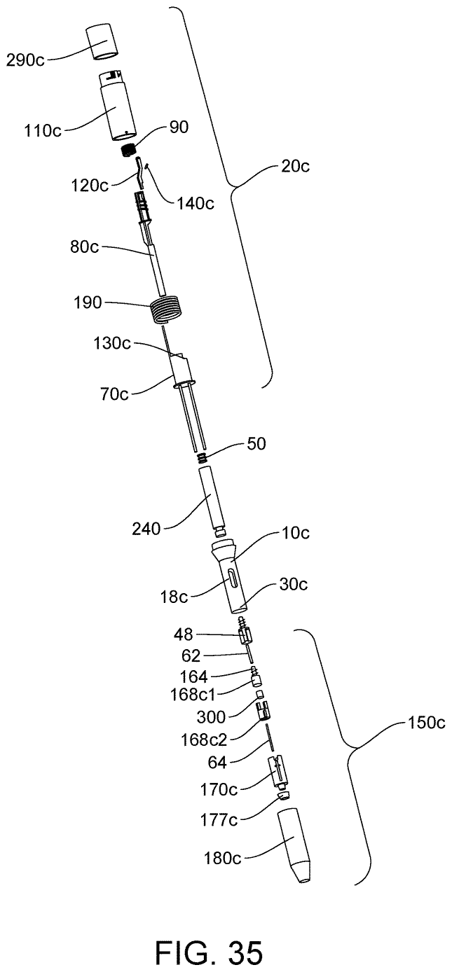

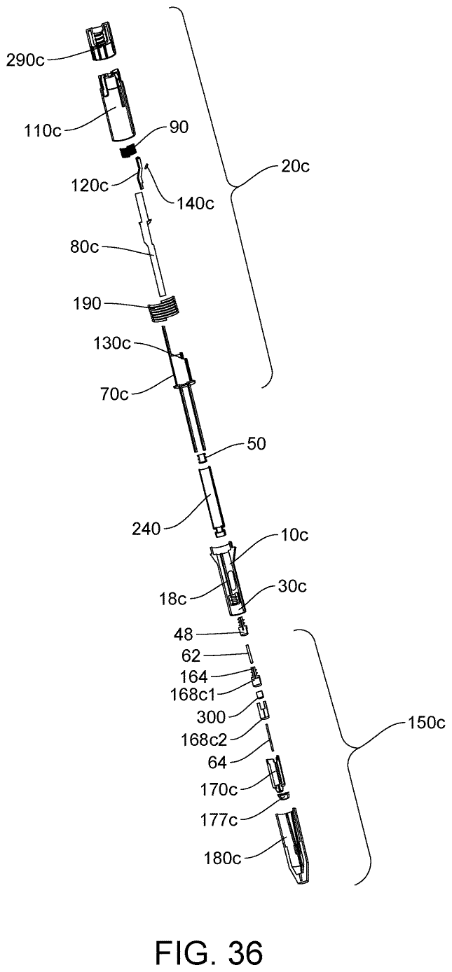

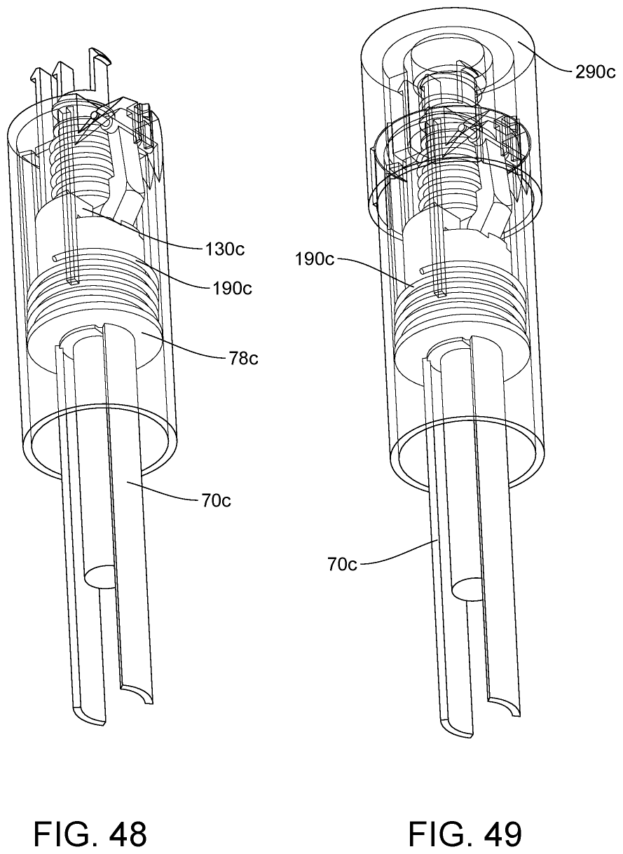

Having reference to FIGS. 35 to 60, a fourth embodiment of injection devices according to the present invention is provided which comprises a number of modifications compared to the third embodiment described above and, in particular, use of a single retention arm 120c and restrainer 130c being a proximal integral extension of sleeve 70c. This embodiment also encompasses the use of replaceable or disposable cartridges.

The injection device comprises a substantially hollow elongated housing 10c having proximal and distal ends, with an actuation mechanism 20c engaged at its proximal end, the actuation mechanism 20c comprising an actuation housing 110c. The elongated housing 10 comprises internally extending longitudinal ribs 220c which defines an inner lumen 30c adapted to receive a formulation cartridge 240 containing, or adapted to contain an active agent for injection into a subject and the formulation chamber also comprises corresponding proximal and distal ends. Stopper 50 is provided at least at the proximal portion of cartridge 240 for retaining active agent therein. Because replaceable or disposable cartridges do not have a barrel connector, according to the present embodiment a needle hub connector 210c is provided as an internally threaded collar supported by radially intruding ribs 220c of elongated housing 10c. Radially intruding extensions 220c also provide slots for sleeve leaves 74c to slide within. Compared to the earlier described embodiments, sleeve 70c may comprise only two opposite sleeve leaves 74c with opposed significant spaces between them, and elongated housing 10c may include the option of a window 18c which coincides with one of the opposed spaces (or two windows, each corresponding with one of the opposed spaces between the sleeve leaves 74c), such that the contents of cartridge 240 (if installed) can be viewed. The proximal end of needle hub connector 210c provides a radially intruding flanged surface upon which the distal end of cartridge 240 may be secured against by the force of plunger 80c pushing against stopper 50.

In the embodiment illustrated in FIGS. 35 to 60, actuation mechanism 20c may comprise an actuation housing 110c, proximal cap 290c, the proximal portion of sleeve 70c, including restrainer 130c as an integral portion of sleeve 70c, and plunger 80c, the distal end of which engages with, or is adapted to engage with stopper 50 of the formulation cartridge. At its proximal end plunger 80c is threaded and extends proximally beyond actuation housing 110c through channel 263c and engages with thread 293c in proximal cap 290c, with at least one driving spring 90 positioned between the proximal end of actuation housing 110c and plunger 80c. Plunger 80c may comprise any suitable means for holding driving spring 90 between plunger 80c and actuation housing 110c, such as ridge 82/86c shown in FIGS. 41, and 44 to 47 formed close to the proximal end of plunger 80c. Actuation mechanism 20c also comprises at least a sleeve spring 190c, as well as longitudinal ribs 330 extending internally from actuation housing 110c, and adapted to engage the proximal end of spring 190c, with the distal end of spring 190c being engaged by the proximal portion of sleeve 70c, such that spring 190c is stressed between ribs 330 and sleeve 70c thereby biasing sleeve 70c towards a distal position.

Proximal cap 290c fits over actuation housing 110c but is not fastened thereto, allowing for adjustment of proximal cap 290c relative to plunger 80c (and stopper 50), and therefore for adjustment of dosage to be delivered.

Elongated housing 10c and actuation housing 110c may engage with one another by any suitable means, such as those known in the art, which will ensure that in use they remain in a fixed positional relationship relative to one another. For example, the mutually compatible surfaces may be welded, pinned, crimped, snapped, screwed or glued together.

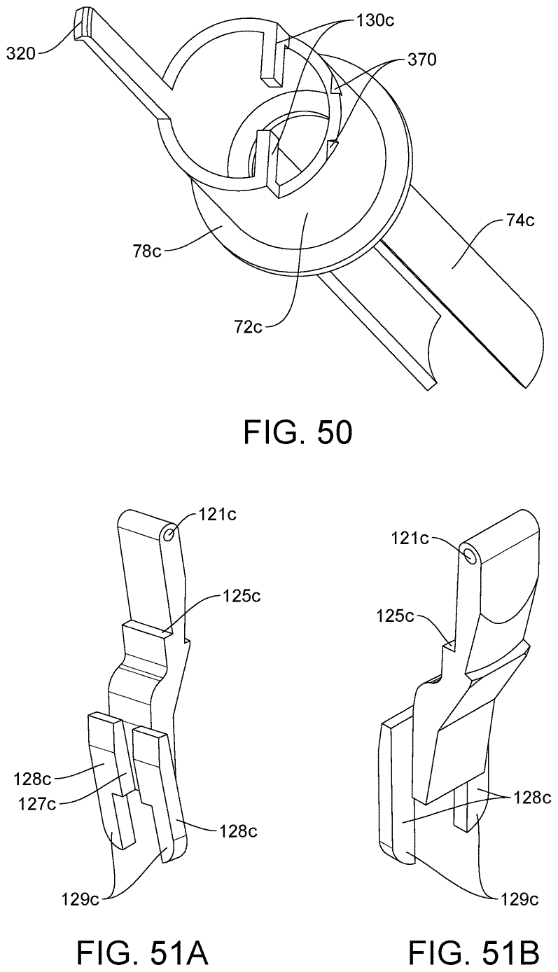

In the specific embodiment illustrated in FIGS. 35 to 60, and particularly FIG. 50, sleeve 70c comprises a hollow cylindrical proximal body 72c with restrainer 130e formed as an integral part of the proximal end of body 72c, and which is housed within actuation housing 110c, with two opposed sleeve leaves 74c extending distally from body 72c, through corresponding slots in the proximal end of elongated body portion 10c and between ribs 220c of elongated housing 10c. The gaps between sleeve leaves 74c may coincide with a window 18c in elongated housing 10c such that the contents of cartridge 240 (if installed) can be viewed. As described earlier, part or all of sleeve 70c may optionally be constructed of a transparent material such that the contents of cartridge 240 may be viewed through window 18c regardless. Sleeve 70c may optionally also comprise surfaces 370 for engaging with retention arm 120c for locking sleeve 70c in a needle sheathing position, as described later, and/or a proximally extending arm for interacting with proximal cap 290c to lock the injection device mechanism until a dosage is set for injection.

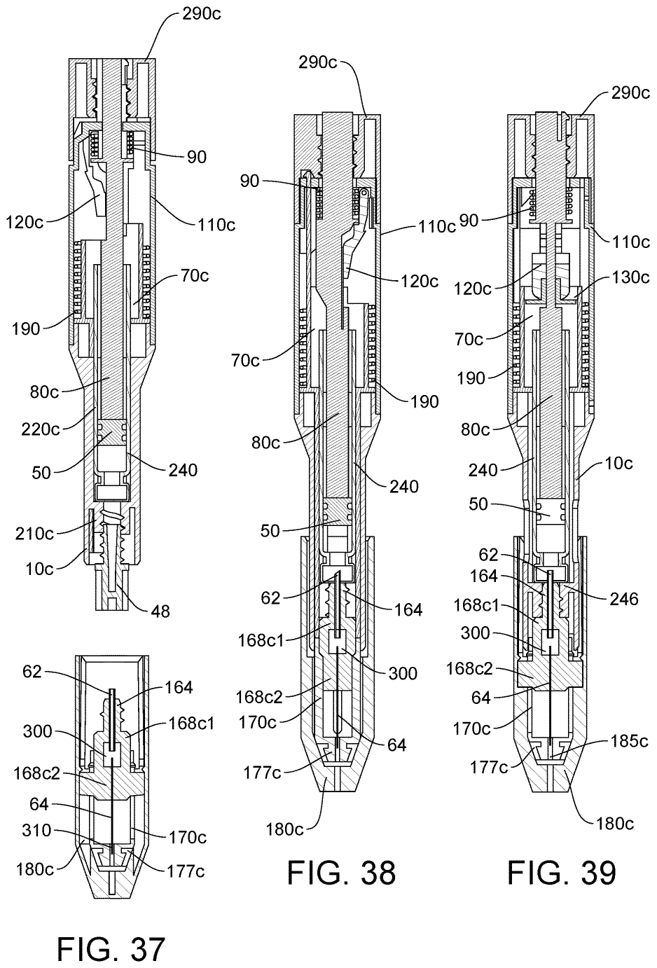

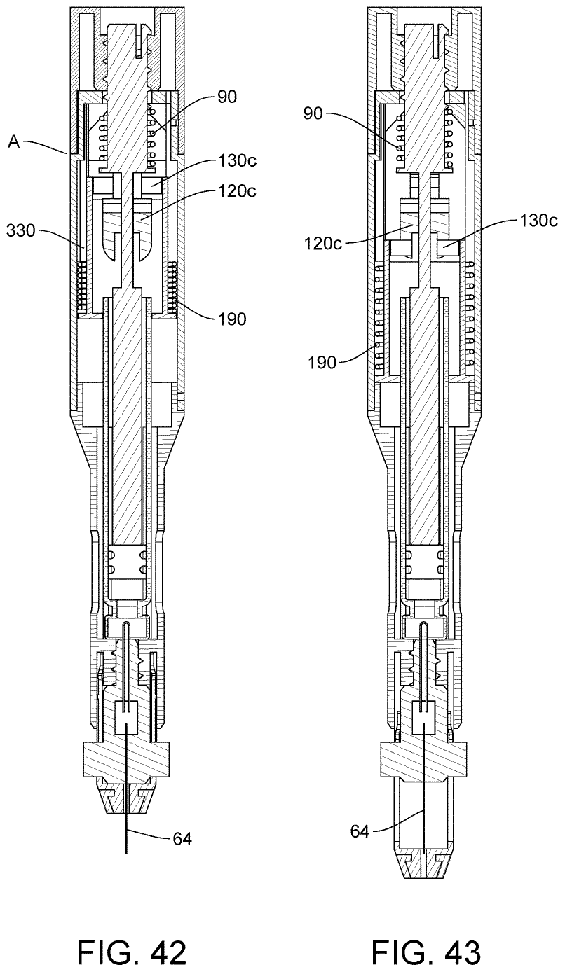

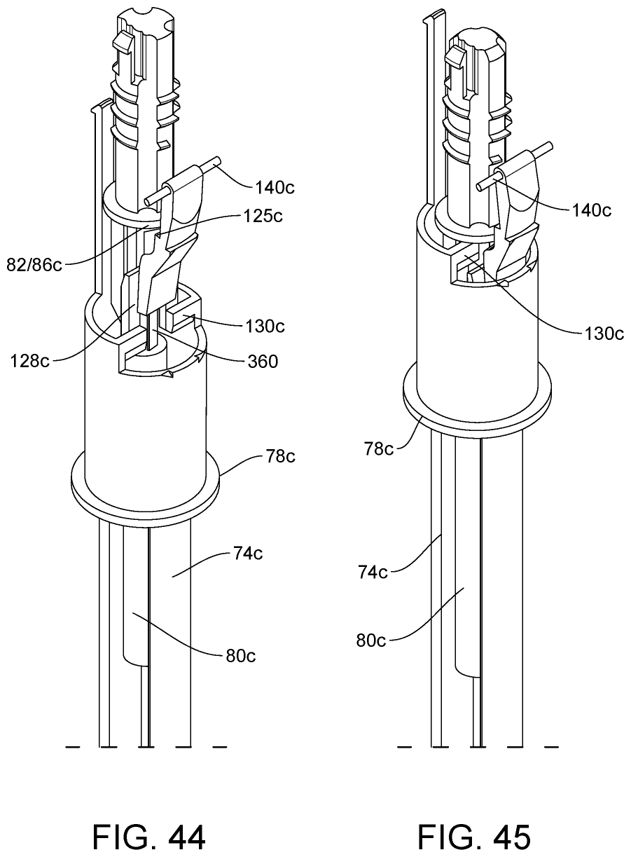

Having reference to FIGS. 37 to 43, when an injection device according to the present invention is put together, with cartridge 240 in place and plunger 80c engaged with stopper 50, with dose set and the device is primed for injection, driving spring 90 is held under stress with plunger 80c held in position by retention arm 120c, with retention arm being held in plunger-engaging position by restrainer 130c which is an integral portion of sleeve 70c and is at least proximally slidable, restrainer 130c being in restraining position when distal (FIGS. 39 and 44) and in releasing position when proximal (FIGS. 42 and 45). In use, restrainer 130c moves proximally (as part of sleeve 70) when sleeve 70 is slid proximally relative to elongated housing 10c by applying distal pressure on the injection device against a surface to be injected, thereby releasing retention arm 120c to deflect away from plunger 80c, allowing driving spring 90 to drive plunger 80c and stopper 50 distally.

Having reference to FIGS. 35-60, and particularly FIGS. 39, 41-47, 50 and 51 and 57, retention arm 120c and restrainer 130c as used in the fourth embodiment are illustrated. In particular, retention arm or toggle 120c as illustrated in FIGS. 39, 41-47 and 51, comprise a single extended ‘arm’ with a pivot/fulcrum point 121c at the proximal end (like a shoulder) adapted to engage a pin or axle 140c (shoulder joint) provided at the proximal end of actuation housing 110c. Alternatively, pin(s) or axle(s) 140c may be formed separately or even form part of retention arm 120c and engage with actuation housing 110c, or corresponding part of the actuation mechanism housing via corresponding joints or fulcrum points 390. Moving distally along the ‘arm’ of retention arm or toggle 120c is a step in or axially oriented ‘elbow’ forming an inwardly directed ledge 125c which, when in plunger-engaging and retaining position, is substantially perpendicular to the axis of plunger 80c and adapted to engage an opposed ridge or ledge 82/86c formed on plunger 80c. Retention arm 120c extends distally beyond inwardly directed ledge 125 (thus forming the equivalent of a ‘forearm’ in the ‘arm’ analogy), ending with a two-fingered ‘hand’ comprising a recess 127c with flanges 128c extending outwardly and distally either side of it and each having sleeve body engaging surfaces 129c at their distal ends. Spring 360 (which may be made of a stiff, elastomeric or springy material, including steel) may be attached to the side of plunger 80c, or even be an integral part of plunger 80c, and push outwardly against a recess in the distal portion of retention arm 120c. Triggering pressure (i.e., the amount of proximally directed force that needs to be applied by sleeve 70c against restrainer 130c, and therefore distal force that needs to be applied against a surface to be injected) using such retention arm is proportional to the ratio a:b between the distance “a” between pivot point 121e and the proximal surface of tab 128c and the adaxial extension distance “b” of the edge of ledge 125c compared to pivot point 121, so for a given bias strength provided by spring 360, the longer “a” is, or the smaller “b” is, the smaller the force required to trigger the device, allowing for less discomfort to a subject being injected

Having reference to the restrainer 130c as illustrated, this is provided as an integral component of sleeve 70c in the form of two flanges extending inwardly from the proximal end of sleeve body 72c, with the two flanges being separated by a gap that allows the body of the ‘arm’ of retention arm 120c to pass through, but not flanges 128c, although other configurations will be readily apparent to those of skill in the art.

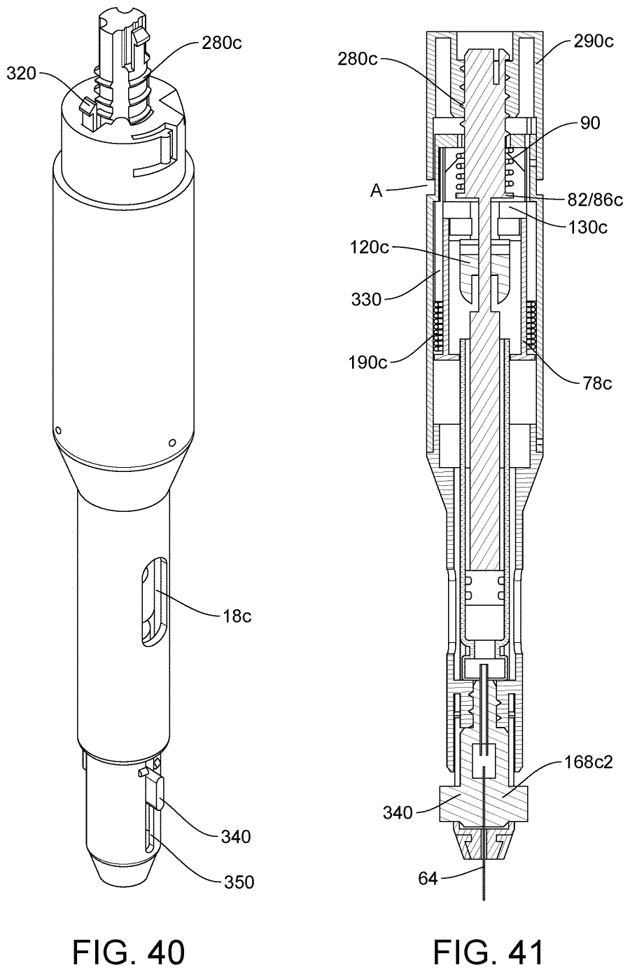

With reference to FIGS. 37 to 43, needle hub 150c, which may be used with any of the second, third or fourth embodiments, may comprise a proximal body 168c1 overmoulded, glued or otherwise attached to a needle 62 extending proximally from it and a distal body 168c2 overmoulded, glued or otherwise attached to a needle 64 extending distally from it, needle 62 being for piercing of the formulation chamber seal 246 and needle 64 being for insertion into a subject for injection. Proximal body 168c1 comprises a proximally formed connector end 164 (as previously described) adapted to engage and connect with barrel connector 210c. Bodies 168c1 and 168c2 are adapted to connect together (and may, for example, be glued, welded, or otherwise coupled), with a filter 300 enclosed between the two bodies for filtration of any particles that may be generated in piercing the prefilled cartridge or prefilled syringe, or any microparticulate matter present in the formulation. Overall body 168c is adapted to fit closely, but slidably within sheath 170c. The proximal end of sheath 170c is adapted to engage sleeve 70c when hub 150c is fully connected with barrel connector 210c. When not in use, hub 150c (whether attached to connector 210c or not) may comprise a cap 180c adapted to clip onto sheath 170c. The distal end of sheath 170c may comprise a guide 185c for securing and protecting needle 64 within the needle hub and during injection. The distal end of sheath 170c may comprise a pad 177c made of softer and/or spongier and/or adherent material than sheath 170c, such as silicone or rubber. Alternatively, the distal end of sheath 170c may be provided with a rough or even spiky surface to assist in keeping the injection device in position during the injection procedure. (for example where reasonably accurate positioning is necessary, such as in the case of intravitreal injections). In further embodiments the distal surface of sheath 170c may be provided with features such as protrusions, ridges and/or other textural elements. In yet further embodiments, the contacting surface of the distal surface of sheath may include a combination of the aforementioned features or materials. Such materials and features may assist in holding the distal surface in position (for example in the correct injection position on the eye surface for intraocular injection) during the injection procedure, and may further assist the clinician in controlling or retaining the position of the eye in a desirable position during the injection. Although not shown, needle hub 150c may comprise a biasing means, such as a spring, within sheath 170c, which urges sheath 170c distally with respect to body 168c and therefore needle 64, to keep needle 64 sheathed when not in use. Sheath 170c may be substantially hollow cylindrical for all or most of its length, although other shapes may be contemplated. In certain embodiments sheath 170c may have a substantially frustoconical distal end. Where sheath 170c and corresponding cap 180c are substantially cylindrical in shape, sheath 170c or cap 180c may comprise means to avoid axial rotation of cap 180c relative to sheath 170c, to allow use of rotational pressure on cap 180c to engage needle hub 150c with barrel connector 210c. Such means may, for example, comprise complementary longitudinal (i.e., distal-proximal extending) ridges and grooves, or introduction of some form of asymmetry in sheath 170c and cap 180c sufficient to stop axial rotation of cap 180c relative to hub 150c, such as previously described herein. As illustrated in FIGS. 37 to 43 and particularly FIG. 40, such means may comprise fins 340 extending from hub body 168c through corresponding slits 350 in sheath 170c and engaging with corresponding slots in cap 180c. In certain aspects cap 180c is adapted to holding the remaining components of needle hub 150c rigidly in place relative to each other to enable handling and attachment of the hub to barrel connector 210c, such adaptations including, for example, clippable flanges, interacting complementary fins and slots (such as illustrated fins 340 extending from hub body 168c through corresponding slits 350 in sheath 170c and engaging with corresponding slots in cap 180c) or grooves and ridges, biasing means, and the like. In certain aspects cap 180c may be closed and have a distal internal void large enough to take a priming volume or dose adjust volume of formulation and the void may also have an absorbent pad to absorb that volume.

In certain aspects a valve, for example a duck valve, as well as the filter 300 may be provided between proximal body 168c1 and distal body 168c2 of the needle hub. Such a valve may be advantageous in inhibiting undesirable leakage of formulation from cartridge 240, such as when residual pressures on the fluid or other forces exist that may move fluid out of the needle at undesirable moments in use of the device.

In certain aspects needle 64 may be made as short as possible (while still allowing for correct injection depth) to reduce formulation flow impedance.

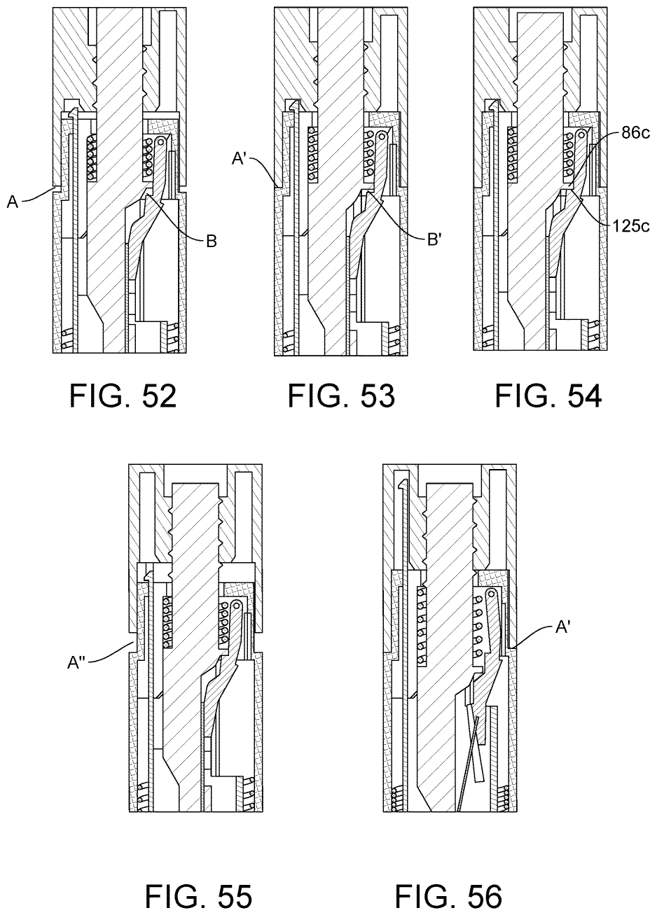

In use, an injection device according to this fourth embodiment, and with reference to FIGS. 37 to 60, the device may be provided as a single-use, pre-packaged sterile device ready for injection, or it may be provided without formulation chamber, either embodiment optionally without needle hub 150c (provided separately in a sterilised packaged form). If a formulation chamber 40 is provided separately, ideally pre-filled, actuation mechanism 20c with sleeve 70c is separated from elongated housing 10c and formulation chamber 40 is inserted into elongated housing 10c with crimped ring 248 and seal 246 resting against the radially intruding flanged surface provided by the proximal end of needle hub connector 210c. Sleeve leaves 74c are then repositioned into elongated housing 10c and actuation mechanism 20c and elongated housing 10e are brought together and fixed in position, at the same time engaging the distal end of plunger 80c with stopper 50. If the formulation chamber is vacuum or voidless filled, this assembly will result in positive pressure within formulation chamber 40. As a result, proximal cap 290c will sit elevated compared to actuation housing 110c, and in a preferred embodiment this results in a gap ‘A’ between the distal surface of proximal cap 290c and the corresponding resting surface of actuation housing 110c (FIGS. 52 and 37), as well as a gap ‘B’ between retention arm surface 125c and plunger ridge 82/86c, gap ‘A’ being smaller than gap ‘B’. Needle hub 150c is then connected to needle hub connector 210c, with plug 48 being removed first, and the proximal end of needle 62 pierces seal 246 and the positive pressure within formulation chamber 40 will result in plunger 80c and connected proximal cap 290c moving distally, with proximal cap 290c coming to rest against the corresponding surface of actuation housing 110c (i.e., gap ‘A” is reduced to a zero gap—gap ‘A” in FIG. 53) and expulsion of formulation from barrel 42 into needles 62 and 64, resulting in self-priming of the system. At the same time, gap ‘B’ between retention arm surface 125c and plunger ridge 82/86c is also reduced to a small gap ‘B”. Having reference to FIG. 54, proximal cap B290c is then turned slightly, reducing gap ‘B’ to 0, thereby calibrating the device. With reference to FIG. 55, further rotation of proximal cap 290c will move it proximally away from its complementary resting surface on actuation housing 110c, creating gap ‘A’” to set the dose travel distance, where it is ready to be triggered and deliver the set dose. Markings may be provided on the external surface of actuation housing 110c which correlate with the proximal extension of proximal cap 290c relative to actuation housing 110c, and therefore the size of gap ‘A’” and the dose equivalent to that gap and, optionally, proximal cap 290c may have one or more windows in its outer surface for viewing such markings and setting the dose, as also described earlier for the third embodiment. The marking for any given dose may extend around the full circumference of the external surface of actuation housing 110c, with regular dose markings associated therewith, to assist in dose setting while rotating proximal cap 290c relative to actuation housing 110c.

This mechanism, which is applicable to not only the fourth embodiment, but also the third embodiment (i.e., embodiments of the present invention employing an actuation housing with a proximal cap, wherein the interaction between the proximal cap and the plunger define the dosing), makes it possible to remove tolerance variability in the specific device in hand, with accuracy of the distance to travel for dose release only being affected by minimal amounts of dimension tolerance variability at dose set point, providing a significant advantage in dose accuracy capability of the device. In addition, a spring may be included between proximal cap 290c and plunger 80c to take up any slack between proximal cap 290c and plunger 80c, to negate any play and corresponding effects on dose variability.

Once the injector is primed and the dose set, restrainer 130c will be in the distal, restraining position, with retention arm 120c held against plunger 80c, with inwardly directed ledge 125c engaging with opposed ridge/ledge 82/86c of plunger 80c, thereby holding plunger in ‘primed’ position against bias provided by first spring 90.

Eye-contacting surface 177c is then placed against the surface to which injection is to be made and pressure applied in a distal direction. This pressure results in sheath 170c sliding proximally into elongated housing 10c and against the distal ends of sleeve leaves 74c and corresponding proximal sliding of sleeve 70c up into actuation housing 110c and against bias provided by second spring 190 being stressed between actuation housing ribs 330 and spring-engaging ridge 78c on the outer circumferential edge of sleeve body 72c. As sleeve 70c moves proximally into the actuation housing restrainer 130c also move proximally. Once restrainer 130c has moved the required distance proximally (as a result of needle 64 reaching the desired injection depth) retention tabs 128c are no longer held against plunger 80c and, as a result, the bias provided by spring 360 deflects the ‘arm’ of retention arm 120c away from plunger 80c (with the abaxial deflection of the distal ends of retention arm 120c being limited by the inner wall of sleeve body 72), breaking the engagement of ledge 125c and ridge/ledge 82/86c and releasing plunger 80c which, under pressure from driving spring 90, is driven distally, resulting in formulation being expelled from the distal end of needle 64 and proximal cap 290c again coming to rest against the corresponding surface of actuation housing 110c (i.e., gap ‘A” is reduced to a zero gap—gap ‘A” in FIG. 56).

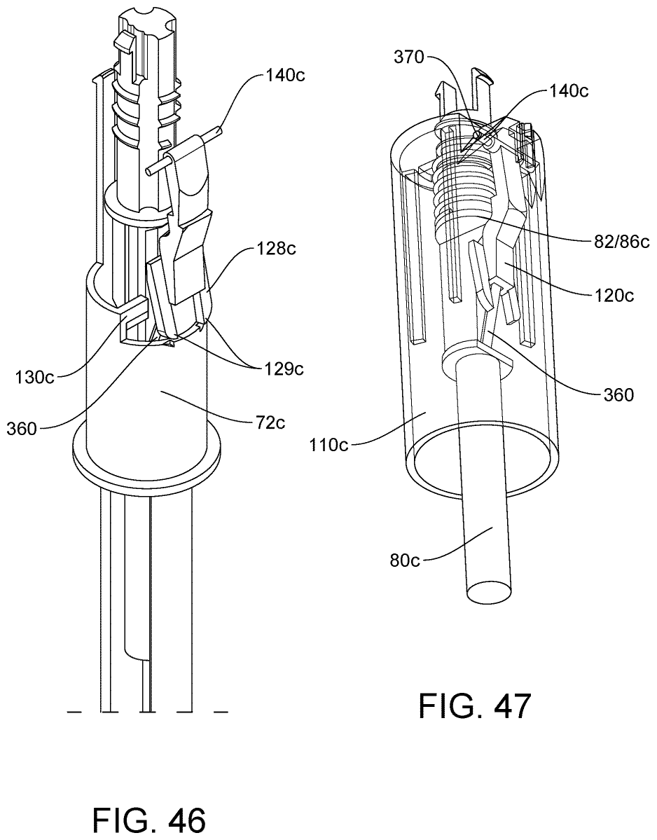

Once injection has been performed, distally applied pressure is eased or removed and, as a result, sleeve spring 190, applying pressure on ridge 78c of sleeve 70c, which is no longer opposed, urges sleeve 70c distally, which in turn results in sheath 170c being urged distally to once again cover needle 64. Also, as a result of sleeve 70c being urged distally, its inner wall no longer limits the further abaxial travel of retention arm 120c, which then deflects further abaxially towards the wall of actuation housing 110c (and radially outward of pivot point 390), with distal edges of retention tabs 129c then engaging with sleeve body 72c, in a preferred embodiment with corresponding recesses/notches 370 formed in the outer rim of the distal end of sleeve body 72c, thereby obstructing potential proximal travel of sleeve 70c and therefore of sheath 170c, thereby locking sheath 170c into a needle covering position, making the device safe (FIGS. 43 and 46). Instead of recesses/notches 370, the proximal surface of sleeve body 72c may comprise alternative means for engaging the distal edges of retention tabs 129c. For example, the proximal surface of sleeve body 72c which makes contact with the distal edges of retention tabs 129c may comprise flexible fingers which more strongly engage the distal edges of retention tabs 129c than a smooth or notched surface. Other means for ensuring strong engagement between sleeve body 72c and the distal edges of retention tabs 129c, to ensure a stronger locking of the sheath into a needle-covering position, will be readily apparent to those skilled in this art.

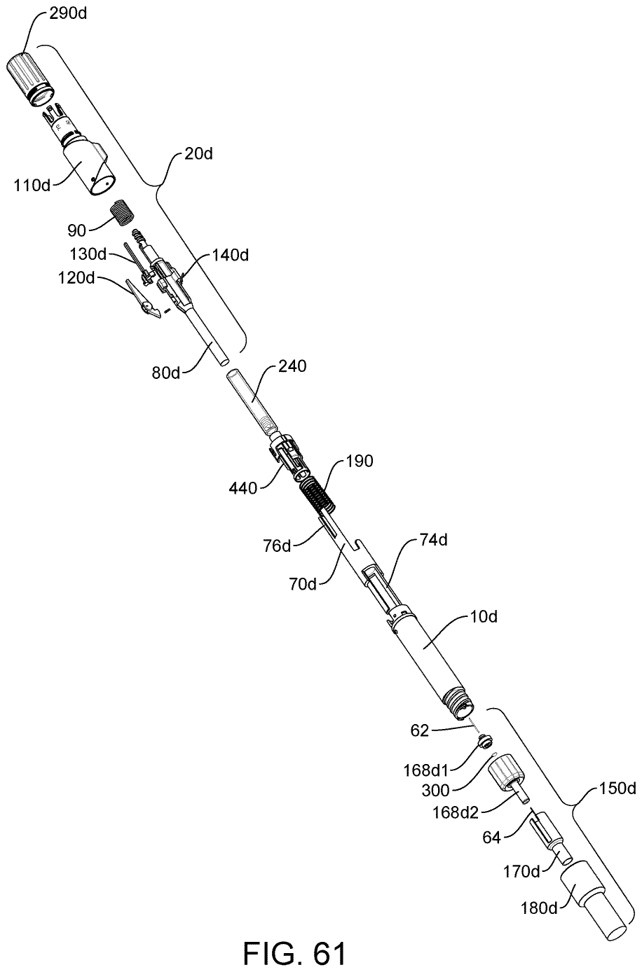

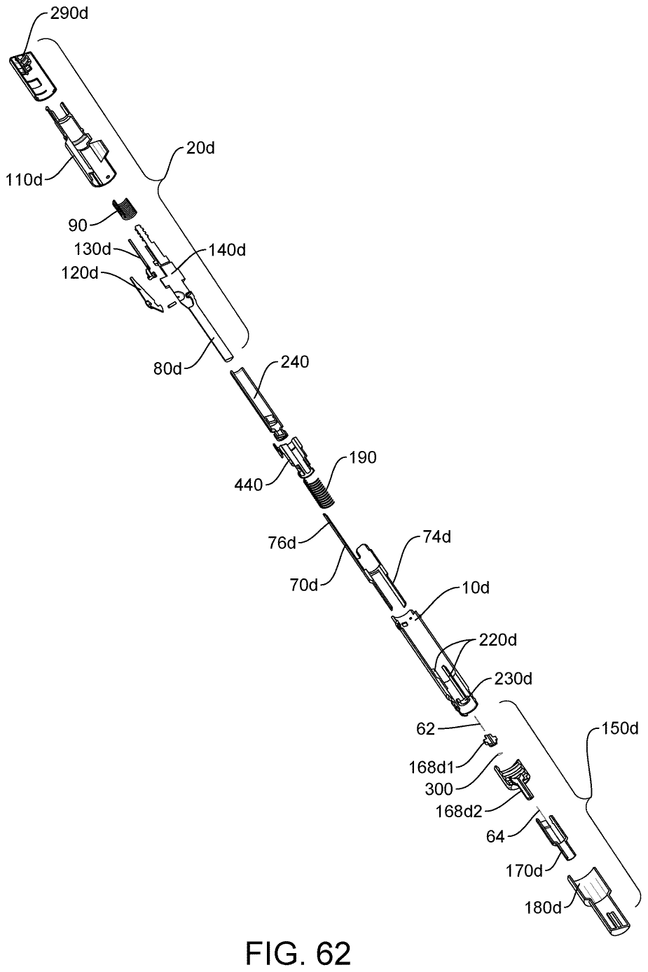

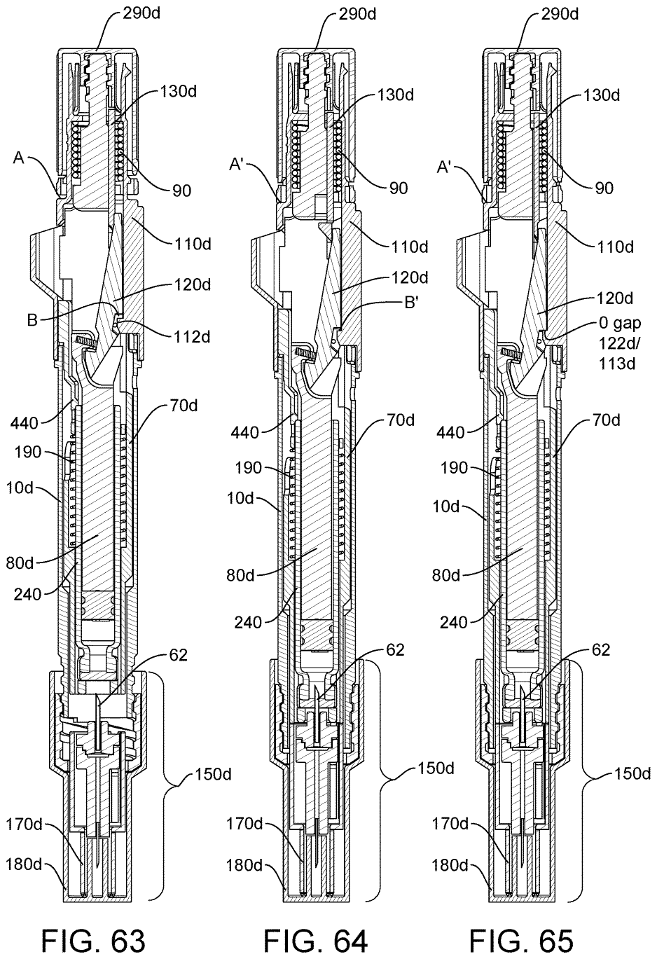

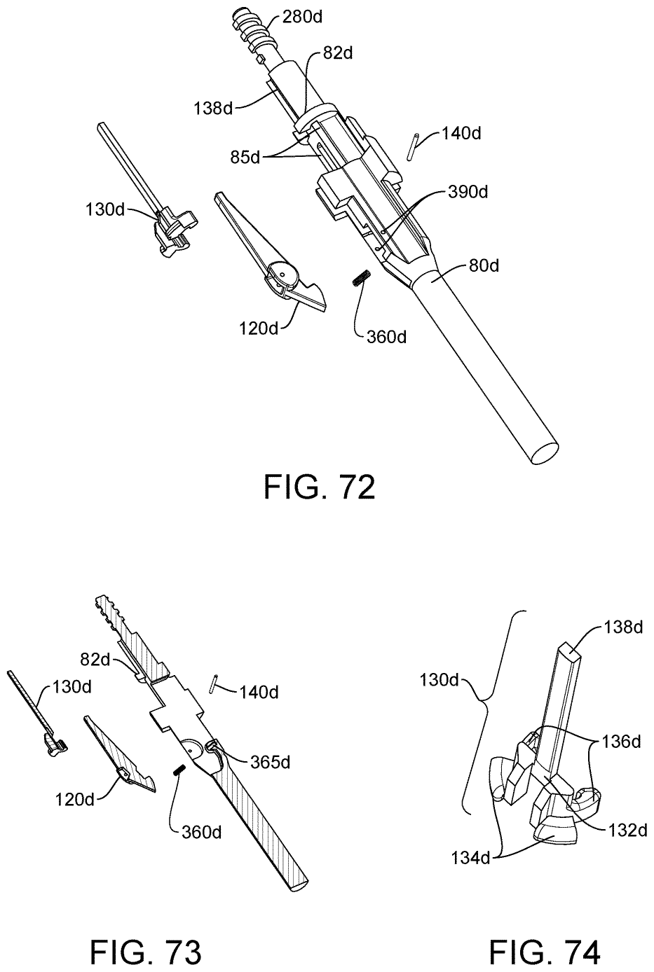

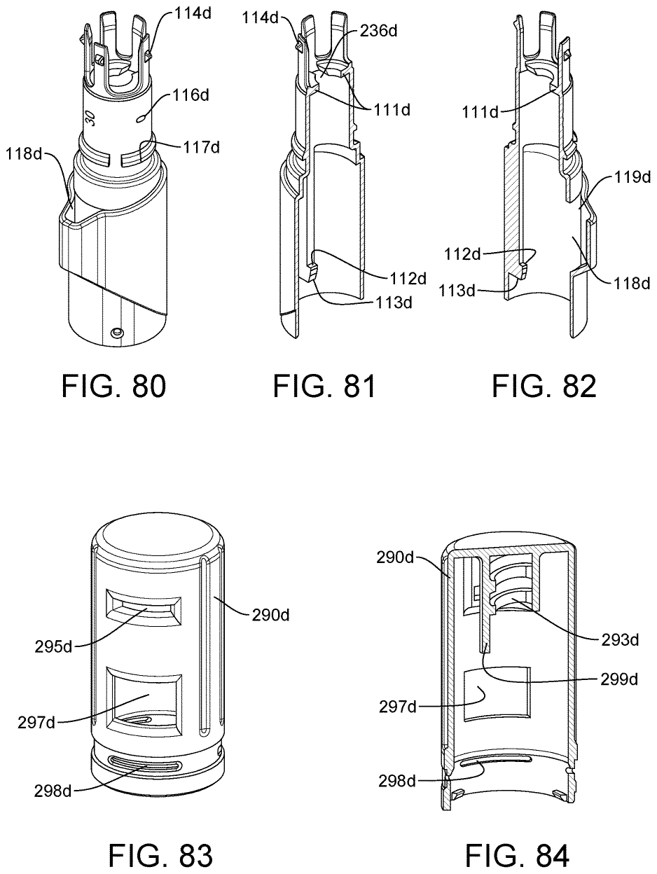

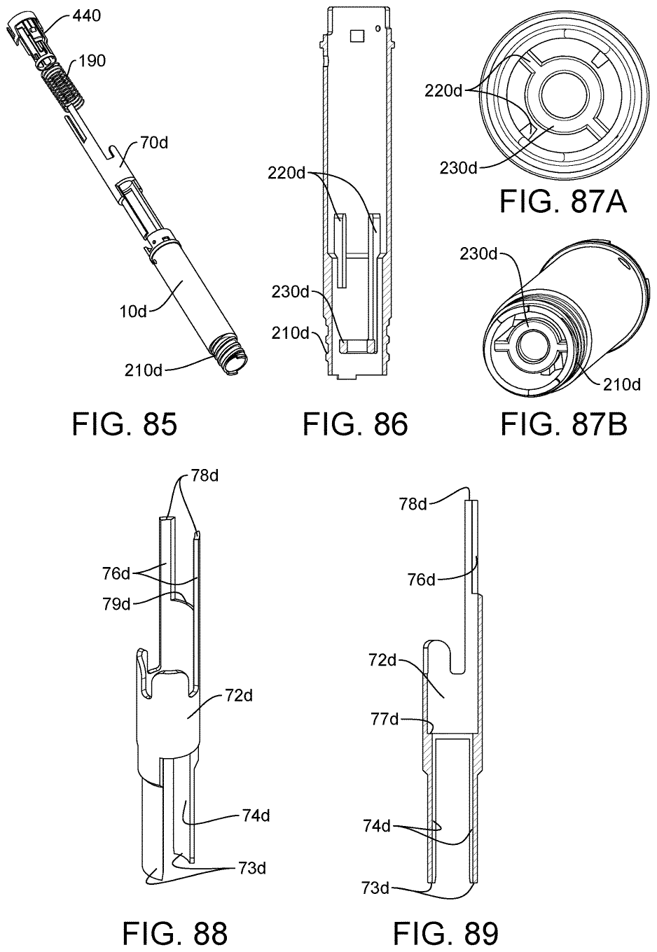

In a fifth embodiment illustrated in FIGS. 61 to 92, actuation mechanism 20d may comprise an actuation housing 110d, which comprises a proximal portion 260d and a distal portion 270d, wherein proximal portion 260d has a narrower diameter and inner bore than distal portion 270d. Distal portion 270d of actuation housing 110d is adapted to house driving spring 90, the proximal half of plunger 80d, a substantial portion of a retention toggle 120d and restrainer 130d. At its proximal end plunger 80d has a threaded extension 280d and extends proximally beyond actuation housing 110d through channel 263d and engages with thread 293d in proximal cap 290d, with at least one driving spring 90 positioned between the proximal end of actuation housing 110d and plunger 80d. Plunger 80d may comprise any suitable means for holding driving spring 90 between plunger 80d and distal surface 111d of the spring retention flange of actuation housing 110d (FIGS. 81 and 82), such as ridge 82d shown in FIGS. 72 and 73 which in that particular aspect is formed at about the proximal third of plunger 80d. In the embodiment illustrated, and with particular reference to FIGS. 63 to 69 and 80-82, actuation housing 110d also comprises a retention ledge with proximal surface 112d adapted to engage retention surface 122d of toggle 120d and a distal surface 113d adapted to engage with lock out surface 128d of toggle 120d. Actuation housing 110d may also comprise a lateral space extension 118d to provide a space for the proximal end of toggle 120d to move into once released (if the proximal end of toggle 120d is needed to be so long—see FIGS. 67 to 69). In certain aspects, an aperture may be provided in the proximal side of extension 118d and/or the actuation housing 110d such that a user may view the proximal end of toggle 120d through that aperture and ascertain that the injection device has been actuated. In certain aspects, indicators configured to indicate one or more of the actuation of the injection device, completion of the injection, confirmation of dose setting, and/or “lock out” of the device may be provided, including haptic indicators, auditory indicators, other visual indicators, one or more electronic signals optionally configured to interface with one or more other devices or applications, or other such indicators known to one having ordinary skill in the art.

In certain aspects, proximal cap 290d fits over actuation housing 110d but is not fastened thereto, threading instead onto thread 280d of plunger 80d (which projects beyond actuation housing 110d) via complementary thread 293d, allowing for adjustment of proximal cap 290d relative to plunger 80d (and stopper 50, not shown), and therefore for adjustment of dosage to be delivered, optionally monitored via markings 116d on housing 110d viewed through window 297d. Dose setting may be facilitated by way of, for example, dose snap settings provided by, for example, dose setting snaps 114d interacting with a dose setting snap recess 295d, or perhaps by way of axially extending furrows spaced radially on the inside of proximal cap 290d, with, for example, one snap per dose setting. Proximal cap 290d may also optionally comprise a window 298d through which a user may see an indicator strip 117d (optionally coloured) when the injection device is primed or when the dose has been delivered.

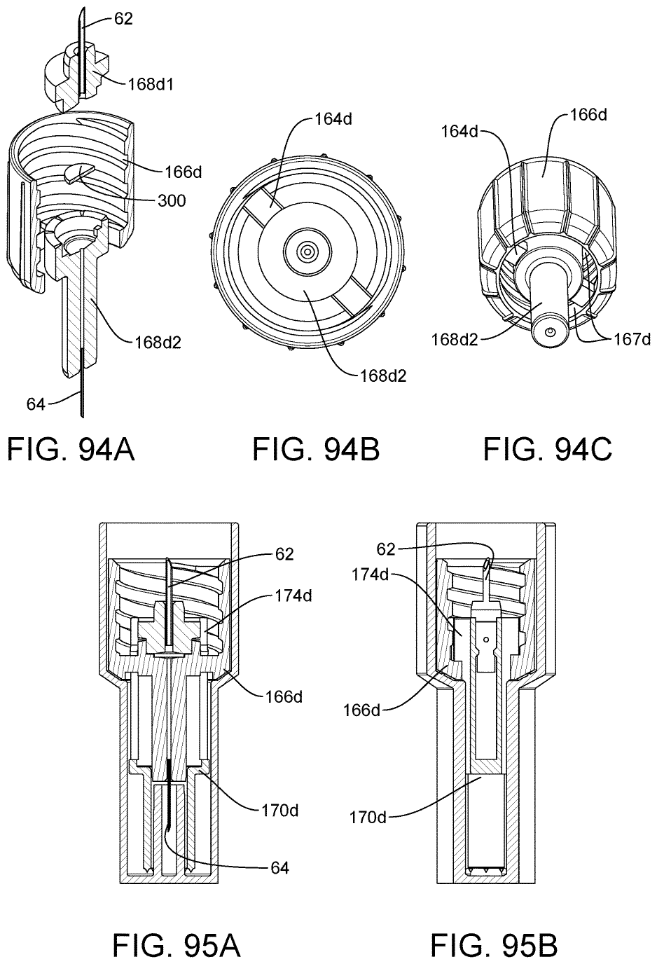

In the embodiment illustrated in FIGS. 61 to 97, and especially FIGS. 85 to 92, elongated housing 10d comprises a hollow cylinder comprising an external thread 210d at its distal end for engaging needle hub 50d. In a further aspect centering ribs 220d which extend proximally from the distal end of housing 10d for at least a portion of the length of housing 10d are provided which are adapted for holding and centering a formulation chamber, pre-filled syringe or cartridge 240 within the elongated housing. One or more of ribs 220d, preferably two or three of said ribs support a cartridge seat 230d provided in the form of a centrally ‘floating’ perforated disc as illustrated in FIGS. 86 and 87. Elongated housing 10d houses sleeve 70d, at least one sleeve spring 190 and, when fitted, formulation chamber 240, all three held in position in the elongated housing by cartridge centering/spring retaining plug 440.

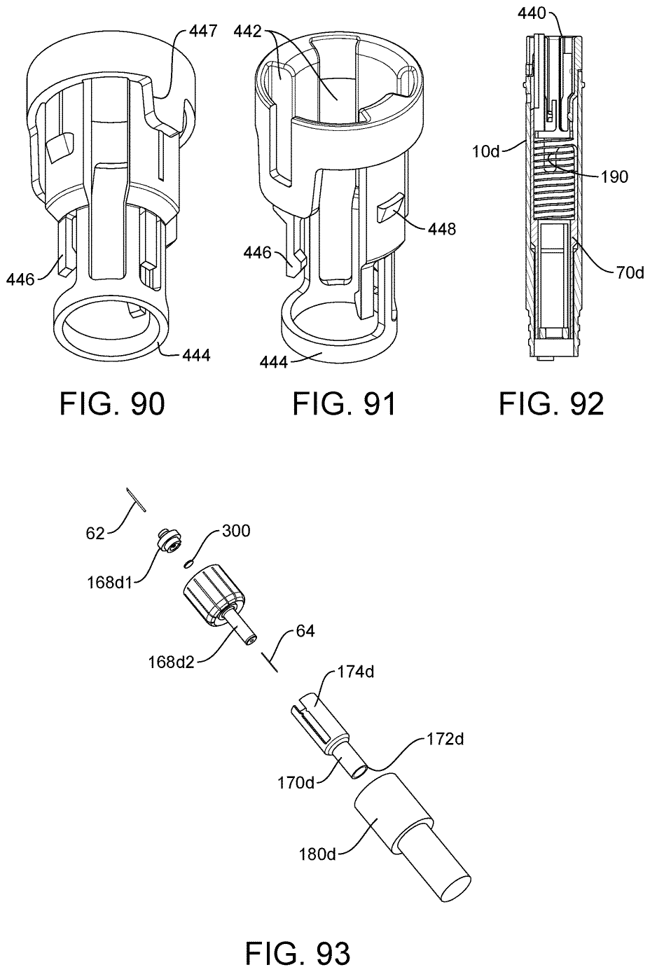

In the embodiment illustrated in FIGS. 90 and 91, cartridge centering/spring retaining plug 440 may comprise slits 442 through which leaves 76d of sleeve 70d may extend proximally, as described below, as well as spring retention/compression ring 444, formulation chamber centering ribs and arms 446, and in preferred embodiments centering/positioning features 447 and snap features 448 for securing plug 440 in position in elongated housing 10d, thereby securing spring 190 and formulation cartridge 240 in position. In certain aspects plug 440 may be a separate object as illustrated in FIGS. 90 and 91, or may be, for example, integrally formed as part of either actuation housing 110d or even elongated housing 10d.

Elongated housing 10d and actuation housing 110d may engage with one another by any suitable means, such as those known in the art, which will ensure that in use they remain in a fixed positional relationship relative to one another. For example, the mutually compatible surfaces may be welded, pinned, crimped, snapped, screwed or glued together.

In the specific embodiment illustrated in FIGS. 61 to 92, and particularly FIGS. 88 and 89, sleeve 70d comprises a hollow cylindrical central body 72d with two opposed sleeve leaves 74d extending distally from body 72d and between ribs 220d (and between housing 10d and chamber 240, if in position), said sleeve leaves comprising distal surfaces 73d adapted to interact with complementary proximal surfaces of needle sheath 170d. Optionally the gaps between sleeve leaves 74d may coincide with a window in elongated housing 10d such that the contents of formulation chamber 240 (if installed) can be viewed, such as window 18c as described in the third embodiment. Alternatively, sleeve leaves 74d or even all of sleeve 70d may be transparent. In an alternate embodiment, the elongated housing may be comprised of a transparent or translucent material to enable visibility of the formulation chamber and/or the internal mechanism contained within the elongated housing. In an embodiment sleeve 70d also comprises leaves 76d extending proximally from body 72d between elongated housing 10d and chamber 240 (if in position) and through corresponding slots 442 in plug 440 (see FIGS. 91 and 92) and comprising proximal surfaces 78d adapted for interacting with complementary surfaces on restrainer 130d and for pushing restrainer 130d proximally when pressure is applied proximally by sheath 170d to the distal surfaces 73d of sleeve 70d.

Having reference to FIGS. 63 to 69 and 77 to 79, when an injection device according to the present invention is put together, with formulation cartridge 240 in place and plunger 80d engaged with stopper 50 (not shown), with dose set and the device primed for injection, driving spring 90 is held under stress with plunger 80d held in position by retention toggle 120d, with retention toggle 120d being held in plunger-engaging position by restrainer 130d which is at least proximally slidable, restrainer 130d being in restraining position when distal (FIG. 77) and in releasing position when proximal (FIGS. 78 and 79).

Having reference to FIGS. 61-92, and particularly FIGS. 72-76, retention toggle 120d and restrainer 130d as used in the fifth embodiment are illustrated. In particular, retention toggle 120d comprises a toggle with a pivot/fulcrum point 121d adapted to engage a pin or axle 140c (shoulder joint) provided about one third of its length from its distal end. Alternatively, pin(s) or axle(s) 140d may be formed separately or even form part of toggle 120d. Pin/axle 140d connects toggle 120d to plunger 80d via corresponding joints or fulcrum points 390d. Toggle 120d also comprises an outwardly directed ledge 122d positioned slightly proximally of pivot/fulcrum point 121d and adapted to engage an opposed ridge or ledge 112d formed on actuation housing 110d. At its proximal end, toggle 120d comprises an axially internal surface 124d adapted to engage a complementary axially external surface 132d on restrainer 130d to hold toggle 120d in an actuation housing-engaging position while restrainer 130d is in its restraining position. Triggering pressure (i.e., the amount of proximally directed force that needs to be applied by sleeve 70d against restrainer 130d, and therefore distal force that needs to be applied against a surface to be injected) using such retention arm or toggle is inversely proportional to a/b where “a” is the distance between pivot point 121d and the proximal surface 124d and “b” is the adaxial extension distance of the edge of ledge 122d compared to pivot point 121d, so for a given bias strength provided by spring 90, the longer “a” is, or the smaller “b” is, the smaller the force required to trigger the device, allowing for less discomfort to a subject being injected. The distances “a” and “b” can be selected for particular injection devices for particular applications. For example, where the injection device is for intraocular injection, a low triggering pressure may be desired, so a value for a/b such that “b is equal to or substantially smaller than “a”, such as a value for a/b of greater than 1, such as between 1 and 1000, may be desired. Toggle 120d may also comprise a lockout ledge 128d adapted to engage with distal surface 113d of the retention ledge of housing 110d and may also comprise a surface 126d which engages with surface 79d of sleeve 70 to provide a further lockout feature, whereby proximal movement of sleeve 70d, and surface 79d pushing against surface 126d of retention toggle 120d, pushing it against the inner wall of the actuation housing, jamming it.

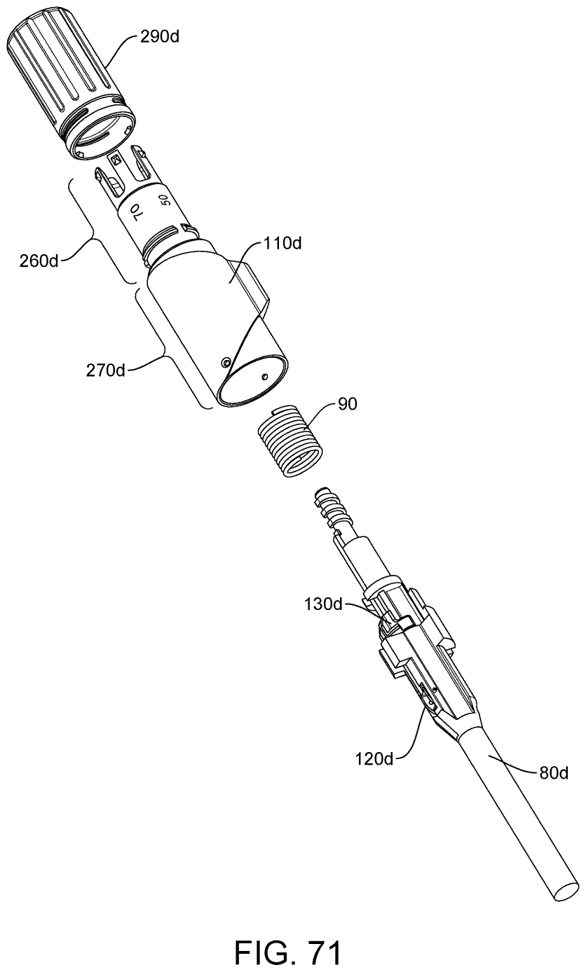

Having reference to the restrainer 130d as illustrated especially in FIG. 74, this is provided as one or more sleeve-engaging surfaces 134d connected to a toggle-retaining surface 132d. In use, restrainer 130d moves proximally when sleeve 70d is slid proximally relative to elongated housing 10d by applying distal pressure on the injection device against a surface to be injected, thereby releasing toggle 120d so as to allow surface 122d to deflect adaxially (under bias from spring 90) from ridge or ledge 112d of actuation housing 110d, allowing driving spring 90 to drive plunger 80d and stopper 50 (not shown) distally. As illustrated in FIGS. 72 to 74, restrainer 130d may comprise means for holding the restrainer in slidable connection with plunger 80d. Such means may comprise, for example as illustrated, arms 136d adapted to clip over ridges 85d on plunger 80d, thereby holding restrainer 130d against plunger 80d in a proximally-distally slidable relationship. In certain aspects restrainer 130d may also comprise a protrusion 138d adapted to pass through aperture 119d in housing 110d and engage with transportation locking bar 299d of proximal cap 290d (to prevent unintentional activation/movement) and slidably positioned in furrow 138d in plunger 80d (FIGS. 71, 72, 81 and 84). In other aspects, restrainer 130 may be formed as a ring fully encircling the plunger, removing the need for clip-on features. In yet other aspects the restrainer may have protrusions like 138d but adapted to slide in corresponding furrows in the actuation housing: many other options for arranging locking protrusions will be apparent to those working in this field given the description above, and illustrations of the intended objective.

With reference to FIGS. 93-95, needle hub 150d, which may be used with any of the second, third, fourth or fifth embodiments, may comprise a body 168d having a proximal body 168d1 having a needle 62 extending proximally from it and a distal body 168d2 having a needle 64 extending distally from it, needle 62 being for piercing of the formulation cartridge seal 246 (if present—if a pre-filled syringe is used instead of a formulation cartridge, there will be no septum to pierce), and needle 64 being for insertion into a subject for injection. Distal body 168d2 is axially centered via ribs 164d within a connector 166d adapted to engage and connect with barrel connector 210d. Bodies 168d1 and 168d2 are adapted to connect together (and may, for example, be snapped together, glued or ultrasonically welded), with a filter 300 enclosed between the two bodies for filtration of any particles that may be generated in piercing the prefilled cartridge or prefilled syringe, or any microparticulate matter present in the formulation. Sheath 170d comprises a distal subject-contacting surface 172d and proximal leaves 174d. Proximal sheath leaves 174d pass slidably through slits 167d between needle hub body 168d2 and connector 166d, and the proximal end surfaces of leaves 174d are adapted to engage the distal surfaces 73d of leaves 74d of sleeve 70d when hub 150d is fully connected with barrel connector 210d. Proximal sheath leaves 174d may also be flexible and comprise snap-tabs for securing the sheath in an interleaved relationship with barrel connector 166d (see FIG. 95). When not in use, hub 150d (whether attached to connector 210d or not) may comprise a removably attachable cap 180d. The cap may be adapted to support, protect, or both support and protect the sheath (and the patient-contacting surface) in its extended position and from contact with external surfaces. In a further embodiment, the removably attachable cap may further have a feature adapted to surround the needle when the removably attachable cap is connected to the needle hub. In certain embodiments, such a feature may be further adapted to receive volume of the formulation expelled during priming of the injector for use. The distal end of sheath 170d may comprise a guide for securing and protecting needle 64 within the needle hub and during injection. Subject-contacting surface 172d may comprise a pad made of softer and/or spongier and/or adherent material than sheath 170d, such as silicone or rubber. Alternatively, the distal end of sheath 170d may be provided with a rough or even spiky surface to assist in keeping the injection device in position during the injection procedure. In further embodiments the distal surface of sheath 170d may be provided with features such as protrusions, ridges and/or other textural elements. In yet further embodiments, the contacting surface of the distal surface of sheath may include a combination of the aforementioned features or materials. Such materials and features may assist in holding the distal surface in position (for example in the correct injection position on the eye surface for intraocular injection) during the injection procedure, and may further assist the clinician in controlling or retaining the position of the eye in a desirable position during the injection. Although not shown, needle hub 150d may comprise a biasing means, such as a spring, within sheath 170d, which urges sheath 170d distally with respect to body 168d and therefore needle 64, to keep needle 64 sheathed when not in use.

As described earlier for another needle hub embodiment, in certain aspects a one way valve, for example a duck valve may be provided between proximal body 168d1 and distal body 168d2 of the needle hub. Such a valve may be advantageous in inhibiting undesirable leakage of formulation from cartridge 240 when positive pressure exists, such as when residual pressures on the fluid or other forces exist that may move fluid out of the needle at undesirable moments in use of the device.

In certain aspects needle 64 may be made as short as possible (while still allowing for correct injection depth) to reduce formulation flow impedance. In other aspects any space between needles 62 and 64 inside the needle hub may be as wide and/or be of the same diameter as needle 62, or may have other geometric adaptations configured to reduce formulation flow impedance. In other aspects needle 62 may have a split channel to allow for the escape of any air trapped in the needle hub during priming and/or injection.

Other optional features of needle hubs according to the invention have already been described above in relation to at least the fourth embodiment and apply to this embodiment equally.

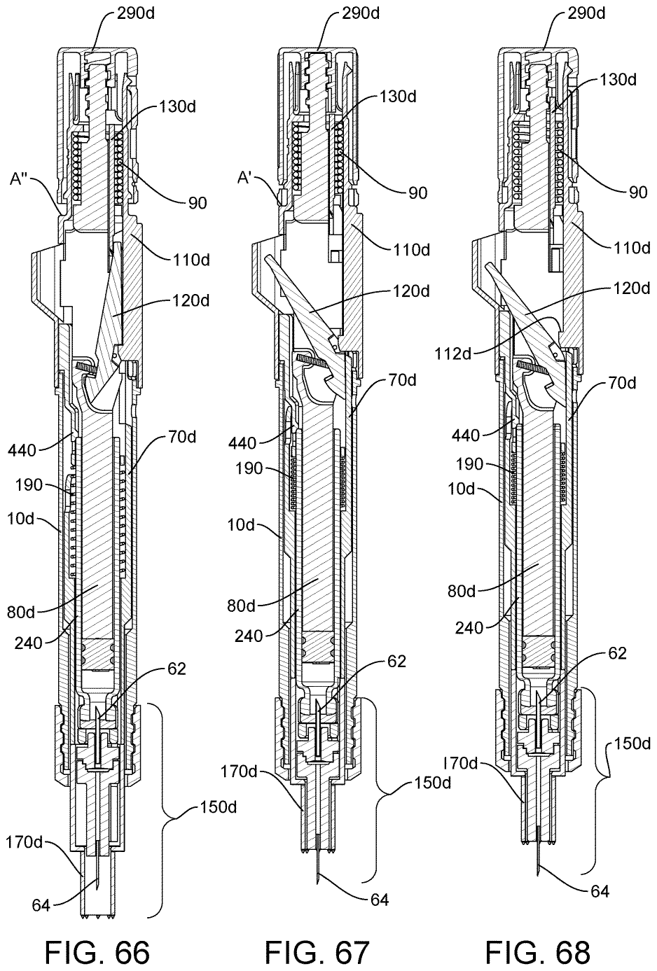

In use, an injection device according to this fifth embodiment, and with reference to FIGS. 61 to 95, the device may be provided as a single-use, pre-packaged sterile device ready for injection, or with a prefilled syringe, or with a cartridge intended to be utilized with multiple patients with separable needle hubs, or it may be provided without formulation chamber, either embodiment optionally without needle hub 150d (provided separately in a sterilised packaged form). If a formulation cartridge 240 is provided separately, ideally pre-filled, actuation mechanism 20d and optionally plug 440 (cartridge-centering arms 446 may be sufficiently flexible to allow cartridge 240 to pass through without the need to remove plug 440) are separated from elongated housing 10d and formulation cartridge 240 is inserted into elongated housing 10d with crimped ring 248 and seal 246 (as illustrated in FIGS. 21 and 22) resting against seat 230d. Plug 440 is then reinserted into the proximal end of elongated housing 10d (compressing spring 190), if it was removed, and actuation mechanism 20d and elongated housing 10d are then brought together and fixed in position, at the same time engaging the distal end of plunger 80d with stopper 50. If the formulation cartridge is vacuum or voidless filled, this assembly will result in positive pressure within formulation cartridge 240. As a result, proximal cap 290d will sit elevated compared to actuation housing 110d, and in a preferred embodiment this results in a gap ‘A’ between the distal surface of proximal cap 290d and the corresponding resting surface of actuation housing 110d (FIG. 63), as well as a gap ‘B’ between toggle surface 122d and proximal surface 112d of the retention ledge of actuation housing 110d, gap ‘A’ being smaller than gap ‘B’. Needle hub 150d is then connected to needle hub connector 210d, and the proximal end of needle 62 pierces seal 246 and the positive pressure within formulation cartridge 240 will result in plunger 80d and connected proximal cap 290d moving distally, with proximal cap 290d coming to rest against the corresponding surface of actuation housing 110d (i.e., gap ‘A” is reduced to a zero gap—gap ‘A” in FIGS. 64 and 65) and expulsion of formulation from cartridge 240 into needles 62 and 64, resulting in self-priming of the system. At the same time, gap ‘B’ between toggle surface 122d and proximal surface 112d of the retention ledge of actuation housing 110d is also reduced to a small gap ‘B” (FIG. 64). Having reference to FIG. 65, proximal cap 290d is then turned slightly, reducing gap ‘B’ to 0, thereby calibrating the device. With reference to FIG. 66, further rotation of proximal cap 290d will move it proximally away from its complementary resting surface on actuation housing 110d, creating gap ‘A’” to set the dose travel distance, where it is ready to be triggered and deliver the set dose. Markings may be provided on the external surface of actuation housing 110d which correlate with the proximal extension of proximal cap 290d relative to actuation housing 110d, and therefore the size of gap ‘A’” and the dose equivalent to that gap and, optionally, proximal cap 290d may have one or more windows in its outer surface for viewing such markings and setting the dose, as also described earlier for the third embodiment. The markings for doses may extend around the circumference of the external surface of actuation housing 110d, to assist in dose setting while rotating proximal cap 290d relative to actuation housing 110d.

This mechanism, which is applicable to not only this fifth embodiment but also the fourth and third embodiments (i.e., embodiments of the present invention employing an actuation housing with a proximal cap, wherein the interaction between the proximal cap and the plunger define the dosing), makes it possible to remove tolerance variability in the specific device in hand, with accuracy of the distance to travel for dose release only being affected by minimal amounts of dimension tolerance variability at dose set point, providing a significant advantage in dose accuracy capability of the device. In addition, a spring may be included between proximal cap 290d and plunger 80d to take up any slack between proximal cap 290d and plunger 80d, to negate any play and corresponding effects on dose variability.

In an alternative embodiment of a priming mechanism needle hub assembly 150d may also comprise a priming spring at the proximal end of proximal body 168d1 which is narrower in diameter than the aperture in seat 230d such that it pushes against cartridge 240 proximally when needle hub 150d is threaded onto barrel connector 210d. In such an embodiment plug 440 may also comprise internally protruding cartridge stops on formulation chamber centering ribs and arms 446 so that when needle hub 150d is threaded onto barrel connector 210d the priming spring pushes cartridge 240 towards the cartridge stops which also moves stopper 50 towards and eventually engaging with the distal surface of plunger 80d before the proximal surface of cartridge 240 reaches the cartridge stops providing a gap ‘x’. Further threading of needle hub 150d onto barrel connector 210d results in plunger 80d pushing on stopper 50 creating positive ‘priming’ pressure in cartridge 240 between the priming spring and plunger 80d (proportional to the spring force and deflection of the priming spring in the hub). Further threading of needle hub 150d onto barrel connector 210d results in further compression of the priming spring and needle 62 piercing septum 44 with formulation then passing through the needle hub to allow pressure equalisation, which also allows further proximal travel of cartridge 240 until its proximal surfaces reach the cartridge stops on plug 440 (i.e., reducing gap ‘x’ to 0), with a concomitant amount of formulation passing through the needle assembly, thereby priming the needle hub 150d. In such a system the positive pressure generated in cartridge 240 between the priming spring and plunger 80d is smaller than would be applied by spring 90 in the embodiment described earlier, reducing delayed pressure-related leakage after piercing of septum 246 (which may occur especially with viscous formulations) and to also prevent loading of the stopper and, thereby preventing pressurization of the fluid and subsequent, sustained loading of the septum during storage. In other aspects needle 62 may have a split channel to allow for the escape of any air trapped in the needle hub during priming and/or injection.

Once the injector is primed and the dose set, restrainer 130d will be in the distal, restraining position, with toggle 120d held so as to have toggle surface 122d engaged with proximal surface 112d of the retention ledge of actuation housing 110d, thereby holding plunger 80d in ‘primed’ position against bias provided by first spring 90.

Patient-contacting surface of sheath 172d is then placed against the surface to which injection is to be made and pressure applied in a distal direction. This pressure results in sheath leaves 174d sliding proximally into elongated housing 10d and against the distal ends 73d of sleeve leaves 74d and corresponding proximal sliding of sleeve 70d and sleeve leaves 76d up into actuation housing 110d and against bias provided by sleeve spring 190 being stressed between spring retention ring 440 and spring-engaging ridge 77d on the inner circumferential edge of sleeve body 72d. As sleeve 70d moves proximally into the actuation housing, proximal surfaces 78d of sleeve leaves 76d also move proximally into the actuation housing 20d, eventually engaging surfaces 134d of restrainer 130d sliding it proximally. Once restrainer 130d has moved the required distance proximally (as a result of needle 64 reaching the desired injection depth) retention surface 132d no longer engages surface 124d of toggle 120d, allowing toggle 120d to rotate about axis 121d (as a result of bias applied by spring 90) and disengaging surfaces 122d and 112d, thereby releasing plunger 80d to be driven distally under the bias provided by spring 90, resulting in formulation being expelled from the distal end of needle 64 and proximal cap 290d again coming to rest against the corresponding surface of actuation housing 110d (i.e., gap ‘A” is reduced to a zero gap—gap ‘A” in FIG. 65).

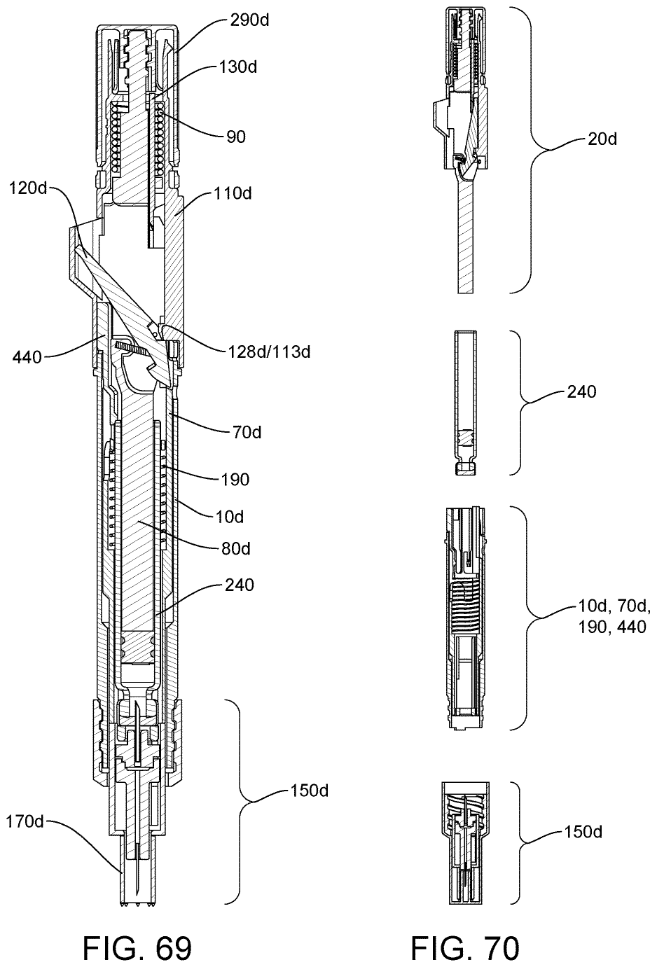

Once injection has been performed, distally applied pressure is eased or removed and, as a result, sleeve spring 190, applying pressure on ridge 77d of sleeve 70d, which is no longer opposed, urges sleeve 70d distally, which in turn results in sheath 170d being urged distally to once again cover needle 64. Also, as a result of sleeve 70d being urged distally, its inner wall no longer limits the further abaxial travel of surface 126d of toggle 120d, which under the bias of spring 360d then deflects further abaxially towards the wall of elongated housing 10d (and radially outward of pivot point 121d), with surface 126d then engaging with surface 79d of sleeve body 72d if sleeve 70d and sheath 170d are urged proximally for any reason, thereby locking sheath 170d into a needle covering position, thereby reducing the risk of needlestick injury by disallowing the distal needle from being re-exposed to the user (FIG. 69). Other means for ensuring strong engagement between sleeve body 72d and the distal edges 126d of toggle 120d, to ensure a stronger locking of the sheath into a needle-covering position, some of which have already been discussed above in relation to other embodiments will be readily apparent to those skilled in this art.

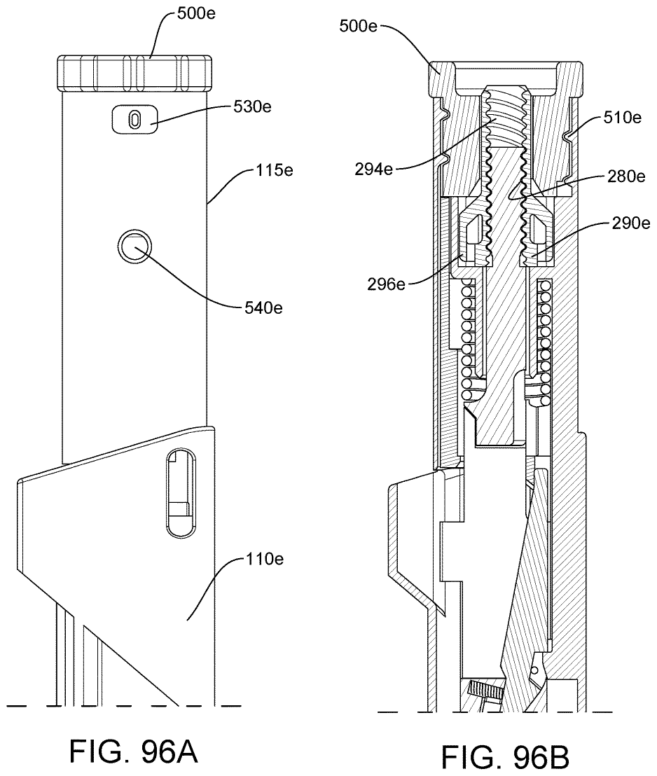

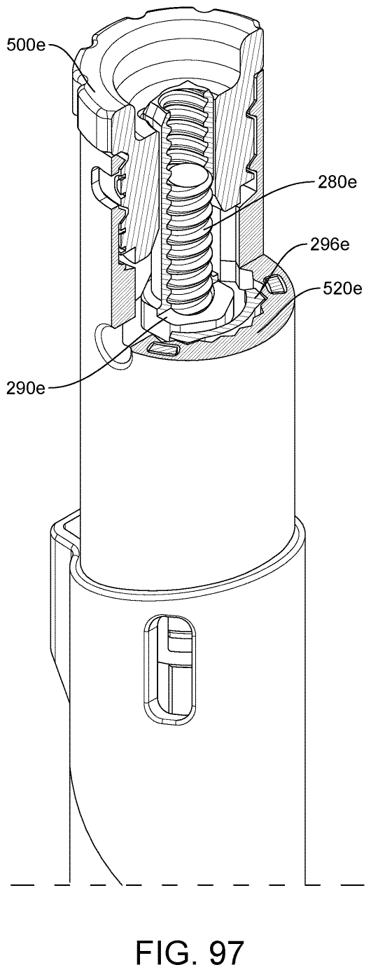

Having reference to drawings 96 and 97 an alternative embodiment relating to dosage mechanisms is illustrated which allows for greater sensitivity and accuracy in setting small doses (for example, doses of 100 μL or less). In this embodiment proximal cap 290d (as described for the fifth embodiment) is replaced by internal dose setting knob 290e with a thread 294e which threads onto thread 280e of plunger 80e (which may be identical to plunger 280d described earlier). The outside surface of internal knob 290e may be of a suitable shape such that external knob 500e slides over internal knob 290e but internal knob 290e and external knob 500e cannot significantly rotate axially relative to one another. Such shape on the outside surface of internal knob 290e may comprise square or oblong cross-section, and/or may involve splines or the like which engage with complementary inner surfaces in external knob 500. External knob 500e comprises an external surface with a thread complementary to a thread 510e on the inside surface of extended section 115e of actuation housing 110e. Thread 510e has a pitch which is significantly larger than the pitch of thread 280e/294e so that when knob 500e is turned anti-clockwise by one full turn, with concomitant turning of internal knob 290e by one full turn, knob 500e travels proximally (compared to the rest of actuation housing 110e) by a significantly greater distance than does internal knob 290e, the benefit being that a large proximal movement of knob 500e results in small proximal travel of internal knob 290e with concomitant small change in amount of dose to be delivered by the device, allowing for greater visual ease in setting small doses for injection by devices according to the present invention. For yet greater convenience, internal knob 290e may comprise flexible extended arms 296e with pips that engage with grooves 520e arranged radially and distally on the inside surface of extended section 115e, as illustrated in FIG. 97 to allow for stepped dosing measurements. As illustrated in FIG. 96A, extended section 115e may also comprise a window 530e for viewing dose settings located on the external surface of external knob 500e, and optionally a window 540e which allows for viewing of whether internal knob 290e has returned to its resting position after actuation, thereby confirming that the device has been actuated and the dose delivered. Parameters such as the number of turns of the dose setting knob to maximum dose, the pitch of the internal dose setting knob thread, diameter of housing 115e, size and number of detents 520e within the inner circumference of housing 115e may all be adjusted to tailor a given device for specific dose volumes and increments and accuracies.

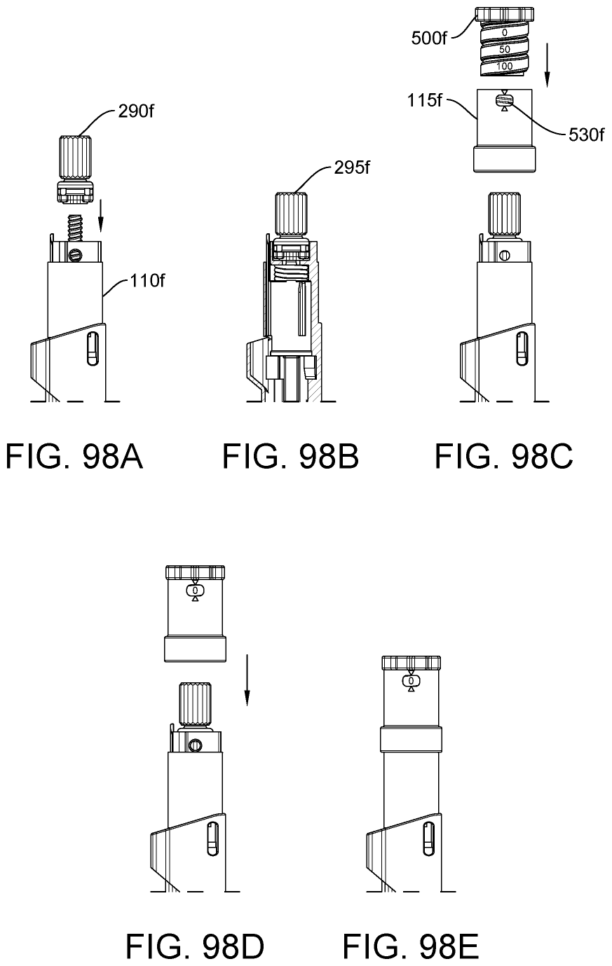

In an alternative to the embodiment illustrated in FIGS. 96 and 97, and as illustrated in FIG. 98, extended section 115e of actuation housing 110e is replaced by a separate dose delivery housing 115f into which external knob 500f threads via complementary threads as described above for components 115e and 500e, with knob 500f being adjusted until a ‘0’ reading is observable in window 530f before fitting external knob 500f over internal knob 290f, which may be the same as internal knob 290e as described above, or which may comprise a multitude of radially arranged proximally-distally extending splines 295f adapted to engage one or more complementary surfaces, such as ridges on the inside of external knob 500f. Dose delivery housing 115f is then connected to actuation housing 110f in a fixed manner/position (i.e., such that dose delivery housing 115f does not rotate or move proximally or distally significantly. The benefit of this embodiment is that, once delivery housing 115f is fixed in position on actuation housing 110f, dose external knob 500f is set to a ‘0’ setting. The ‘0’ setting is achieved by having the retention toggle surface 122d engage with actuation housing surface 112f (i.e. there is no gap between the two, they are in contact), then internal knob 290f is rotated until it contacts actuation housing 110f, or nearest detent (which could result in very small gap between 295f and 110f) or, alternatively, a very small gap may be present between surfaces 122f and 112f (because plunger 80f is raised and spring 90 compressed, raising retention toggle 120e off the ledge) which can be controlled as desired by the thread pitch on 290f. Assembly 115f/500f is then fitted over internal knob 290f as the spline pattern on 500f matches the spline pattern 295f, and the system is correctly set at 0.

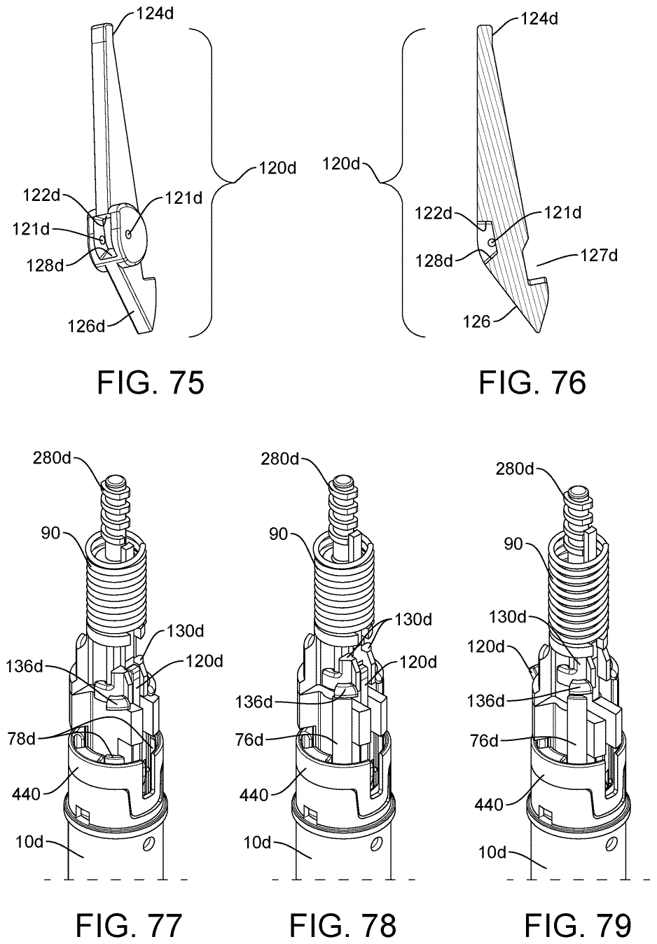

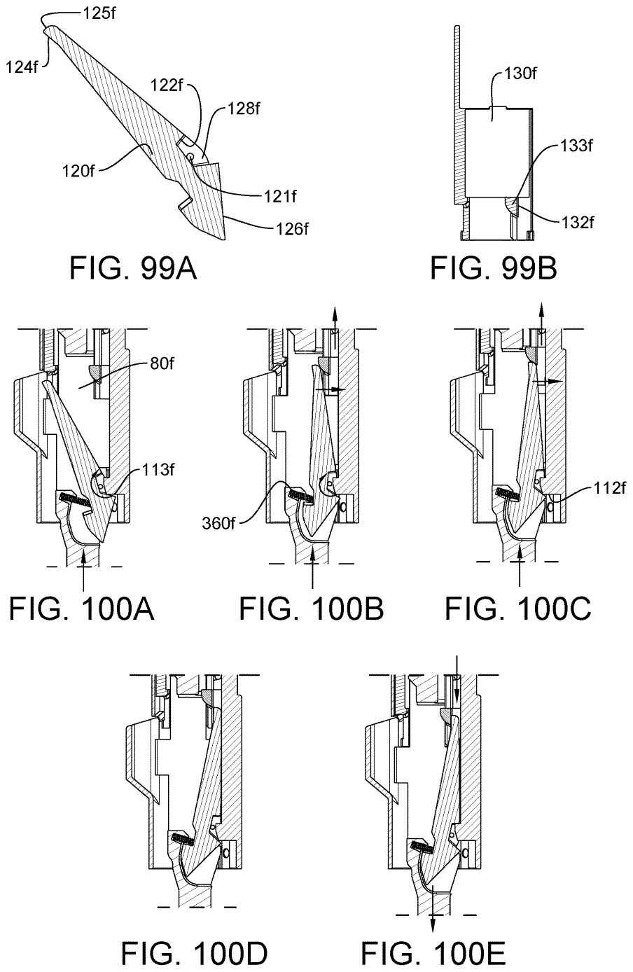

With reference to FIGS. 99 and 100, a self-setting retention arm mechanism is described, which is applicable to the embodiments having a retention toggle. In such an aspect, a slightly modified retention toggle 120f has a bevelled or rounded surface 125f and has a slightly elongated surface 128f compared to surface 128d of the retention toggle illustrated in FIGS. 75 and 76. Also, as well as a retaining surface 132f (corresponding with retaining surface 132d in FIG. 74), restrainer 130f comprises a bevelled/rounded surface 133f adapted to engage slidably with surface 125f of retention toggle 120f. Once the actuation mechanism is assembled, proximal pressure applied to plunger 80d/e/f pushes surface 128f proximally until it engages surface 113d/e/f on the inside of actuation housing 110d/e/f. Continued proximal pressure applied to plunger 80d/e/f causes retention toggle to pivot about pivot axis 121f, against bias applied by spring 360f and aligning retention toggle 120f with plunger 80d/e/f and bringing bevelled/rounded surface 125f of retention toggle 120f in contact with surface 133f of restrainer 130f, such that continued proximal pressure on plunger 80d/e/f results in abaxial pressure by surface 125f being applied to surface 133f, such that restrainer 130f is pushed proximally, against bias applied by spring 90 (not shown), until surface 125f and surface 124f have moved abaxially relative to retaining surface 132f at which point restrainer 130f is again free to move distally under its own weight (or optionally under bias applied to the proximal surface of restrainer 130f) until restrainer 130f comes to rest on a complementary surface on plunger 80d/e/f. Removal of proximal pressure on plunger 80d/e/f then allows the bias of spring 360f and spring 90 to apply pivotal pressure on retention toggle 120f such that surface 124f is pushed against retention surface 132f, stopping further pivoting of retention toggle 120f and also allows plunger 80d/e/f, restrainer 130f and retention toggle 120f to move distally under bias from spring 90 until surface 122f of retention toggle 120f comes into contact with surface 112f of actuation housing 110f, stopping further distal movement of the assembly such that the actuation mechanism is now in an activated configuration, ready for triggering.

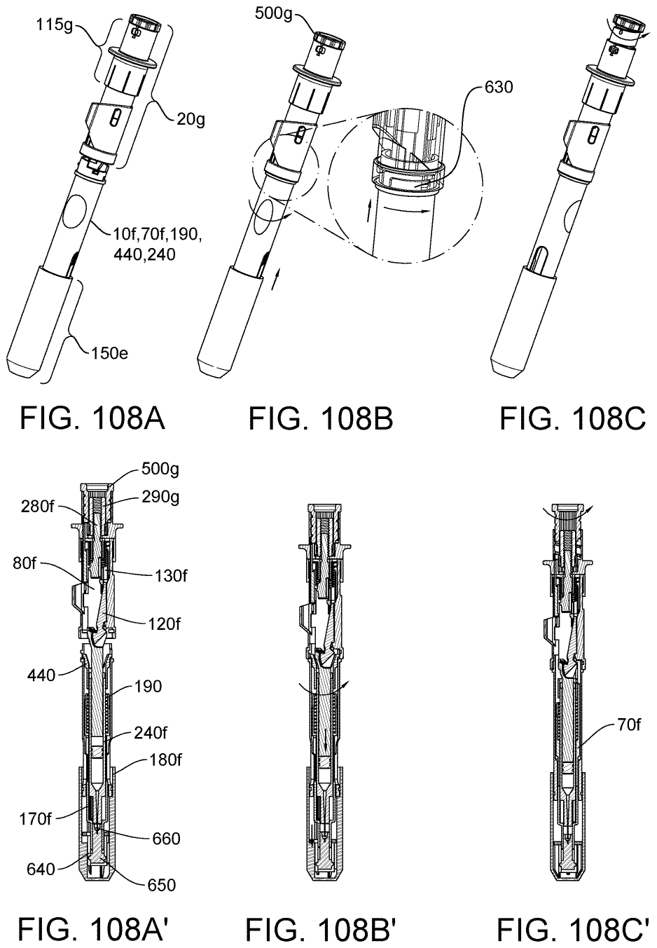

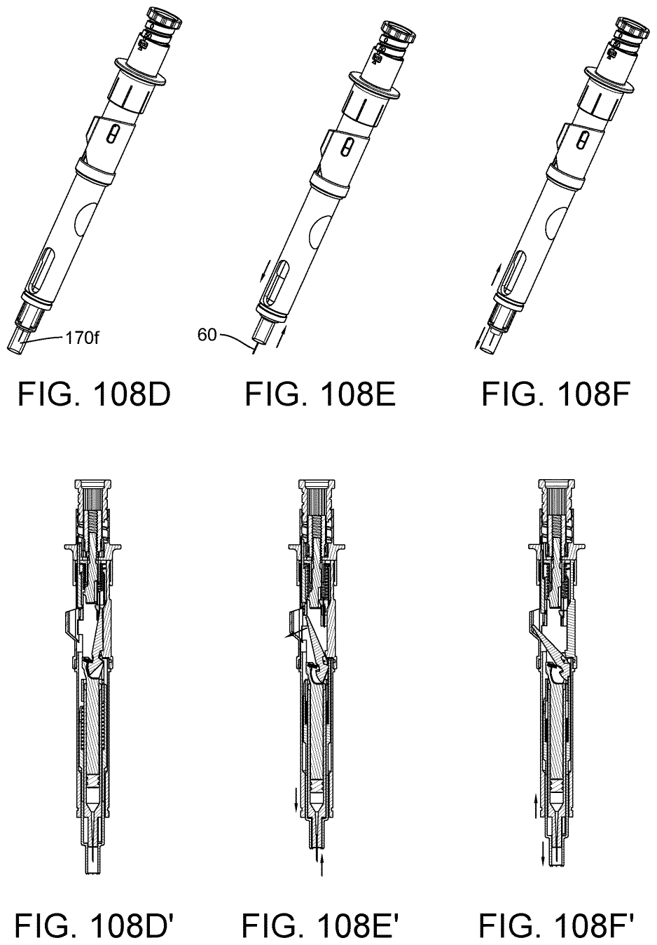

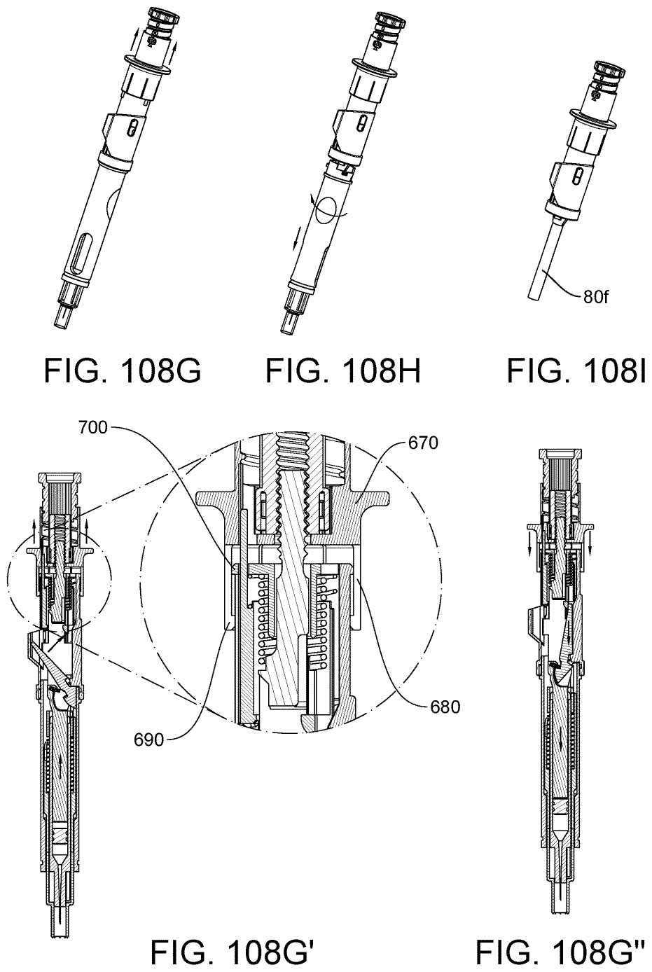

Having reference to FIG. 108, with a particular focus on FIG. 108 G′, in the context of the dosage mechanism illustrated in FIG. 98 and described above, the self-setting retention arm mechanism illustrated in FIGS. 99 and 100 and described above lends itself to an injection device which can be re-set for multiple injections using the same actuation mechanism, with the same elongated housing (with or without replacement of formulation chamber(s)), or replacing the elongated housing (again, with or without replacement of formulation chamber(s)). In the specific embodiment illustrated in FIG. 108, the formulation chamber 240f is formed integrally with the elongated housing 10f, being connected to inside of the wall of the elongated housing by axially inwardly extending ribs. However, the mechanism described here can operate with embodiments as described earlier involving separate formulation chambers, such as syringes and cartridges, which may be replaceable. Sleeve 70f is slidably positioned between formulation chamber 240f and the wall of the elongated housing 10f, and sleeve 70f is biased distally by sleeve spring 190 (held in place against sleeve 70f by spring retention ring 440) and is adapted to engage the distal end of restrainer 130f. Restrainer 130f in turn engages with the proximal surface 124f of toggle 120f which is connected to plunger 80f via pivot point 121f as described above. In the specific embodiment illustrated in FIG. 108, needle sheath 170f is formed separately to sleeve 70f, but is adapted to engage the distal end of sleeve 70f and therefore when sheath 170f is positioned against a target portion of anatomy to be injected and the injection device urged distally, sheath 170f pushes against sleeve 70f which slides proximally as a result and in turn pushes restrainer 130f proximally to release proximal surface 124f or toggle 120f which is then free to pivot from its unactuated position to its actuated position, thereby releasing plunger 80f to be impelled distally by spring 90, as described previously. Whilst the embodiment specifically illustrated in FIG. 108 has a sheath 170f formed separately to with sleeve 70f, the mechanism described here may also operate with needle sheaths that are integrally formed with sleeves 70. Furthermore, whilst the embodiment specifically illustrated in FIG. 108 has an integrally formed needle (which is glued or overmoulded into position), the mechanism described here may also operate with formulation chambers which have their own needle assemblies, or with elongated housing with detachable/replaceable needle hubs as per any of the embodiments described previously. Importantly, the injection device illustrated in FIG. 108 comprises a modification to the dose delivery mechanism illustrated in FIG. 98 and described above, the modification being that dose delivery housing 115g, as compared to dose delivery housing 115f illustrated in FIG. 98 and described above, has a circumferentially outwardly extending flange or flanges 670 which may be used to pull the dose delivery mechanism proximally to effect the toggle re-set as illustrated in FIG. 98 and described above. Such a flange is not necessary, and dose delivery housing 115f or a different modification to that shown in FIG. 108 may be equally effective (dose delivery housing 115f may for example simply be pulled proximally, and perhaps have a rough external surface to assist in this). Dose delivery housing 115g may optionally comprise a distally extending extension 680 as illustrated, which fits slidably over the actuation housing, optionally clipping over the extension housing and being secured there (such that pulling of the dose delivery housing off the injection device is impeded) via ledge 690 extending inwardly from dose delivery housing 115g and ledge 700 extending outwardly from actuation housing 110f.

Instead of circumferentially outwardly extending flange or flanges 670 to pull the dose delivery housing proximally, a twist ring could be positioned below a modification of 115g or 115f such that the dose delivery housing rests on the twist ring which engages the actuation housing via a complementary thread system such that, when the twist ring is rotated axially, it moves proximally, pushing the dose delivery housing proximally to effect the re-setting mechanism described above. When released, the twist ring may return to its starting position under pressure from the driving spring 90, or may remain in any position between it fully twisted position or its starting position, if a non-zero dose is set on the dose delivery mechanism, but the twist ring will return to its starting position if and when the actuation mechanism is triggered. With such a configuration, the pitch of the actuation housing to twist ring thread can be customised to move axially at a desired distance per angle of rotation, but the pitch must not inhibit the twist ring from rotating back to its starting position. Alternatively, or in addition, a spring could be provided to drive the twist ring back to its start position after every reset action.

Having reference now to the procedure illustrated in FIG. 108, this relates to a multi-use injection device adapted for use and replacement of pre-assembled elongation housings, having pre-filled formulation chambers (which may or may not be integrally formed with the elongated housing. As shown in FIGS. 108A and 108B (and corresponding cross-section views 108A′ and 108B′), a pre-assembled elongated housing 10f (with components 70f, 190, 440 and 240) is fitted onto plunger 80f and the elongated housing then brought together with actuation housing 20g (already pre-zeroed as described above in relation to the mechanism illustrated in FIG. 98) and connected thereto by any suitable means, such as by a twist lock mechanism as illustrated in FIG. 108B, which results in plunger 80f contacting the formulation chamber stopper, pushing it distally and associated priming of distal needle 60 (see FIG. 108B′; white arrow pointing downwards). A dose is then set by adjusting dose setting knob 500g (FIGS. 108C′ and 108C′), needle cap 180f is then removed (FIGS. 108D and 108D′), sheath 170f is positioned against a surface to be injected (not shown), distal pressure is applied to the injection device, resulting in sheath 170f, sleeve 70f and restrainer 130f sliding proximally with toggle 120f being released to pivot to its actuated position and plunger 80f being driven distally by spring 90 to deliver formulation from formulation chamber 240 (FIGS. 108E and 108E′). Once injection has been performed, distal pressure on the injection device is removed, and sleeve spring 190 then biases sleeve 70f and sheath 170f distally such that sheath 170f once again fully enshrouds needle 60 (FIGS. 108F and 108F′). At the same time toggle 120f further pivots to its fully pivoted position such that surface 126f is in contact with the inside wall of elongation housing 10f, and surface 113g (equivalent of surface 113f as illustrated in FIG. 100) of actuation housing 20g and surface 128f of toggle 120f engage, to enforce lock out, whereby proximal sliding of sleeve 70f and sheath 170f (and exposure of needle 60) is impeded. Before or after removal of the assembled, but now used elongated housing assembly, the actuation housing may then be re-activated as illustrated in FIGS. 108G, 108G′ and 108G″, with reference also to FIGS. 99 and 100 and the associated description above in relation to the mechanism. As illustrated in FIGS. 108G and 108G′, proximal pressure on optional flange(s) 670 assists in the proximal motion of dose delivery housing 115g to slide plunger 80f proximally and pivot toggle 120f back to its unactuated position and, as shown in FIGS. 108G″ and 98, relaxation of proximal pressure on dose delivery housing 115g results in sleeve 70f sliding distally and allowing restrainer 130f to again engage surface 124f of toggle 120f to hold it in its unactuated position, resulting in the actuation housing being re-set. If the assembled elongated housing was not removed before re-setting of the actuation housing, it can now be removed to provide the actuation mechanism ready for attachment of a fresh, pre-assembled elongation housing having a fresh, pre-filled formulation chamber, ready for performing another injection, as per the procedure described above.

Also having reference to FIGS. 108A to 108C (and corresponding cross-section illustrations 108A′ to 108C′), a modified needle cap assembly is illustrated which assists in avoiding loss of formulation from the formulation chamber or engaged needle and support and protect the needle when not in use. While the needle illustrated in FIG. 108 is integral with the elongation housing (being glued, overmoulded with, or otherwise fixed to the elongation housing), the cap configuration illustrated may be used with other needle assemblies, including replaceable needle hubs as also described earlier, optionally with adaptation(s) that ensure that the needle hub can be attached to the elongated housing (for example, to ensure that the cap does not slip or slide axially around the needle assembly body when the needle assembly is attached to the elongated housing by a twisting action). The cap 180f illustrated in FIGS. 108A to 108C (and FIGS. 108A′ to 108C′) has a tip section 640 distal to the elongated housing and which is at least partially held within the proximal portion of cap body 180f in a slidable relationship, such that tip section 640 can slide at least distally if not also proximally within the cap body 180f. Tip section 640 also has a seal 650 made of a pierceable material, such as a spongy, resilient or rubbery material positioned inside in a manner such that the proximal end of sheath 170f can slide between the inner wall of tip section 640 and seal 650, and seal 650 engages with needle 60 in a sealed manner (for example, needle 60 pierces seal 650 at least partially) and, at the same time, may assist in supporting needle 60 while not in use. When needle 60 is in fluid communication with a formulation chamber under pressure (such as when the formulation chamber stopper is biased distally by plunger 80f during priming), this pushes formulation through needle 60, which then results in the formulation pushing seal 650 distally, which in turn results in tip section 640 being slid distally in relation to cap body 180f, as well as priming of needle 60. Seal 650 may optionally include a space 660 at the proximal end of the seal (which may remain within the distal end of sheath 170f even after priming) which acts as a reservoir for accepting fluid expelled from needle 60, such as during priming.

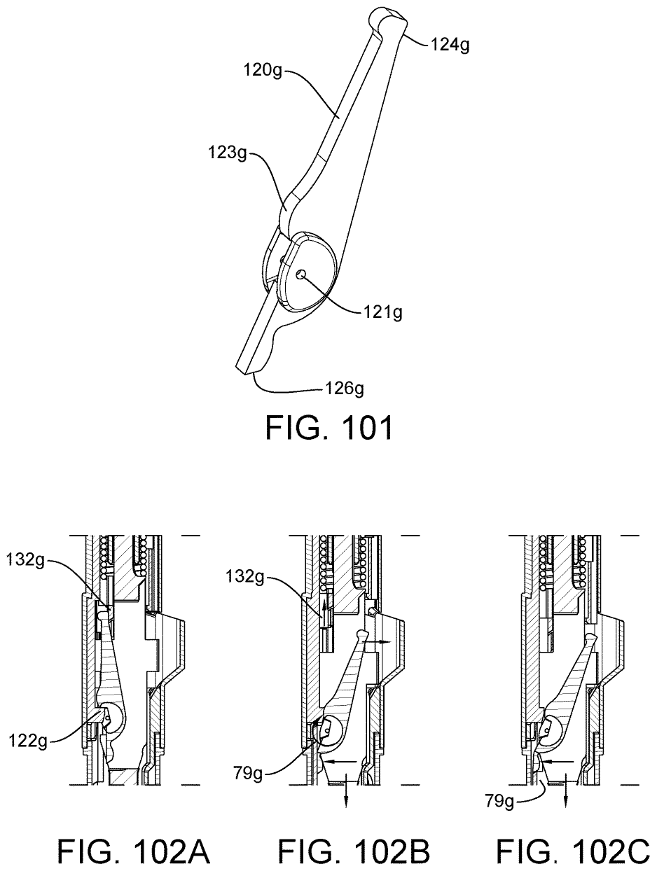

Having reference to FIGS. 101 and 102, a cammed retention toggle 120g is illustrated, which does not require a biasing spring 360d/e/f for pivoting about axis point 121g once triggered. Once the actuation mechanism is triggered by proximal movement of restrainer 130g, and therefore uncoupling of surfaces 124g and 132g, plunger 80g and connected retention toggle 120g are driven distally by spring 90, resulting in pressure between surface 123g of retention toggle 120g and surface 112g of actuation housing 110g, resulting in retention toggle 120g pivoting about axis 121g as a result of the rounded nature of surface 123g until surface 123g has pivoted adaxially to the extent that plunger 80g is free to travel distally. As a result of the pivoting of retention toggle 120g about axis 121g, lock out extension 126g pivots abaxially and comes into contact with and is pushed against the inner surface of sleeve surface 79g. According to one embodiment, lock out extension may be flexible, or at least be flexibly attached to retention toggle 120g and, as a consequence, it may be bent as a result of it being pushed against the inner surface of sleeve surface 79g but, when triggering pressure is removed from sleeve 70g, surface 79g slides distally (as a result of bias by spring 190 on sleeve 70g, not shown) and beyond lock out extension 126g, which then deflects into the space vacated by surface 79g. Further proximal pressure applied to sleeve 70 will then result in surface 79g coming into contact with, and its further proximal travel being stopped by, lock out extension 126g. Thus, if a needle hub with sheath is attached, proximal travel of the sheath is also inhibited by lock out extension 126g, thereby keeping needle 64 sheathed after the mechanism has been actuated.

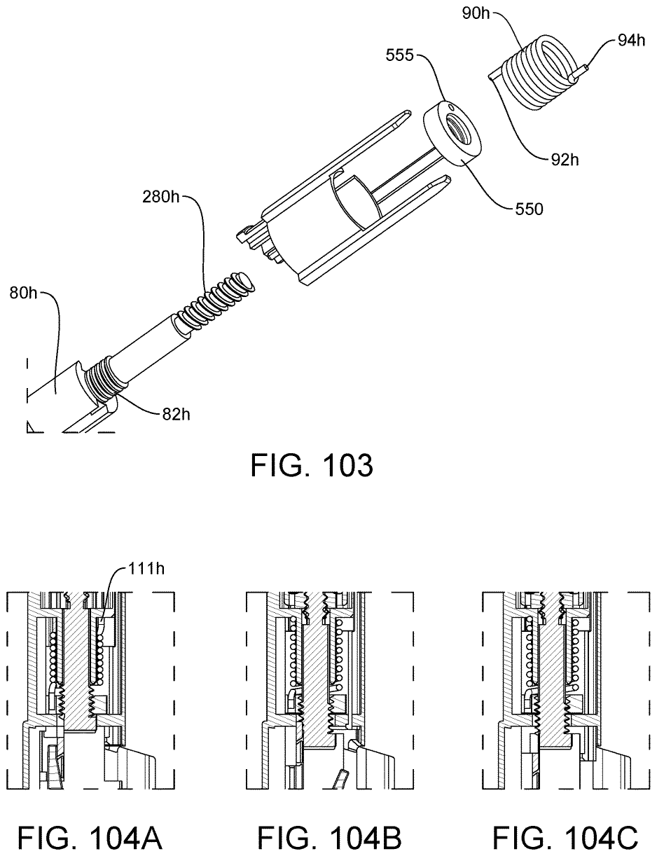

In all embodiments of injection devices according to the present invention, driving spring 90, which urges plunger/plunger 80 distally (and therefore provides the force which expels formulation from formulation cartridge or chamber 40 or 240) may be replaced by any other suitable biasing mechanism. For example, and as illustrated in FIGS. 103 and 104, a torsion spring may be used instead. In such an embodiment torsion spring 90h has proximal pin 94h which engages with a complementary slot 111h in the proximal end of actuation housing 110h and distal pin 92h which engages with complementary slot 555 in torque nut 550. Torque nut in turn engages with thread 82h on plunger 80h. When the actuation mechanism is assembled and ready for triggering, torsion spring 90h is wound up and has stored energy. When the mechanism is triggered, as described previously, torsion spring 90h unwinds, turning torque nut 550, in turn driving plunger 80h distally. The relationship between the torque diameter (defined by the position of slot 555 and the rotational axis of torque nut 550) and the pitch of thread 82h determines the mechanical advantage of the system.

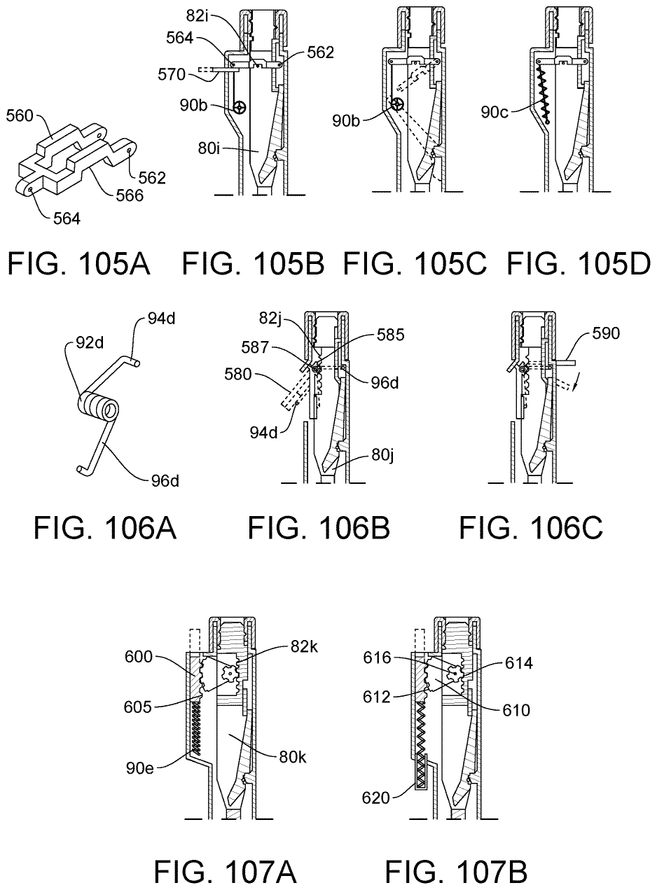

FIGS. 105 to 107 illustrate other possible mechanisms for pushing plunger 80 distally, optionally with mechanisms for securing the device and/or reducing the load on the plunger until intended to be actuated.

For example, as shown in FIG. 105, the spring or other resilient pulling means (for example a constant force spring 90b or extension spring 90c) may be offset from plunger 80i and exert bias on a lever arm 560 via point 564, the lever arm also may fit around plunger 80i and be connected to the actuation housing via axle point(s) 562. Lever arm 560 also has recessed space(s) 566 for engaging plunger pin 82i (which may extend on both sides of plunger 80i such that, when the spring or other resilient pulling means exerts its bias on point 564 of the lever arm 560, lever arm pivots distally about axle point(s) 562, and exerts distal pressure on plunger pin 82i, and therefore on plunger 80i. The leverage or mechanical advantage obtained using such a mechanism is provided by the ratio of distance between 564 and 562 and the distance between 566/82i to 562. Optionally removable tab 564 may hold lever arm in position while the device is idle (for example while being in storage or being transported), thereby inhibiting actuation of the device and exertion of pressure on plunger 80i (and formulation cartridge 240, if installed) until desired.

As shown in FIG. 106, a torsion spring 90d may be offset from plunger 80j and exert bias on a gear lever 580 having gear cogs 585 at its axle end which interact with corresponding teeth on plunger 80j. Torsion spring 90d has a body 92d, which fits around the gear lever axle 587, an arm 94d which engages the adaxial side of gear lever 580 and an opposite arm 94d which engages a complementary surface, slot or edge on the actuation housing opposite the point of connection of gear lever 580. Arm contact distance from axis of gear lever 580 versus diameter of gear teeth engaging with plunger 80j provides the mechanical advantage. When torsion spring 90d exerts its bias on gear lever 580, deflecting gear lever 580 abaxially as a result of rotation about its axle 587, gear lever cogs 585 engage corresponding teeth on plunger 80i, pushing plunger 80i distally. The abaxial deflection of gear lever 580 may also act as a visual indicator showing that the device has been actuated. Torsion spring 90d may be already wound, and gear lever 580 being under bias therefrom when the injection device is assembled or, optionally, the device may comprise a switch, such as lever 590, which may be operated to wind torsion spring 90d when ready to use the injection device.

As shown in FIG. 107, an alternative geared mechanism is illustrated where a compression spring 90e, offset from plunger 80k engages with a cogged rack 600 having cogs 605. Cogged rack 600 engages the abaxial side of gear 610 which also has an adaxial side that engages plunger 80k. The abaxial side of gear 610 engages the toothed rack cogs with corresponding cogs 612, and the adaxial side of gear 610 has cogs which engage corresponding cogs 82k on plunger 80k. When compression spring 90e exerts its bias on cogged rack 600, it pushes it proximally, thereby also pushing the abaxial side of gear 610 proximally making gear 610 pivot about its axis 616, in turn pushing its adaxial side and cogs 614 distally, thereby also pushing plunger 80k distally. Compression spring may already be compressed when assembled (and, for example, stored) or, optionally, the injection device may comprise a compression button 620 for compressing spring 80k when the injection device is ready for use.

The alternative biasing mechanisms illustrated in FIGS. 103 to 107 have the advantage of providing greater mechanical advantage, and therefore applying greater injection force with smaller biasing devices, such as springs, which may be particularly advantageous when injecting high viscosity formulations.

In certain other embodiments of injection devices according to the present invention, unintended early actuation of the device may be avoided through a locking mechanism that interacts with a restrainer as described herein in such a way as to stop it moving longitudinally along a distal-proximal axis. Such a mechanism may include, for example, a pin or stiff slip that passes through the actuation mechanism housing and into a corresponding berth or slit formed in the side or other part (such as an extension) of the restrainer. Alternatively, the outer perimeter of the restrainer, or more preferably a distal extension of the restrainer within the diameter of the restrainer, may comprise a partial ridge/flange with lateral abaxial groove that engages with a flange extending adaxially from the actuation housing and, in such an embodiment, clockwise or anticlockwise movement of the proximal cap, such as when setting the dose, will result in disengagement of that relationship, freeing restrainer to move. An exemplary locking mechanism is illustrated in FIGS. 35-60, where sleeve 70c has a proximally extending rod 320 that extends through slot 380 in the proximal end of actuation housing 110c, and which is then impeded by corresponding blocking surface 410 formed in a channel 420 in the underside of proximal cap 290c when in place. When proximal cap 290c is turned to calibrate the system and set the dose, as described earlier, surface 410 no longer covers the proximal end of rod which is now free to move into channel 420, thereby unlocking the device and allowing sleeve 70c to move proximally. Other such releasable locking mechanisms can be readily conceived by those working in this field.

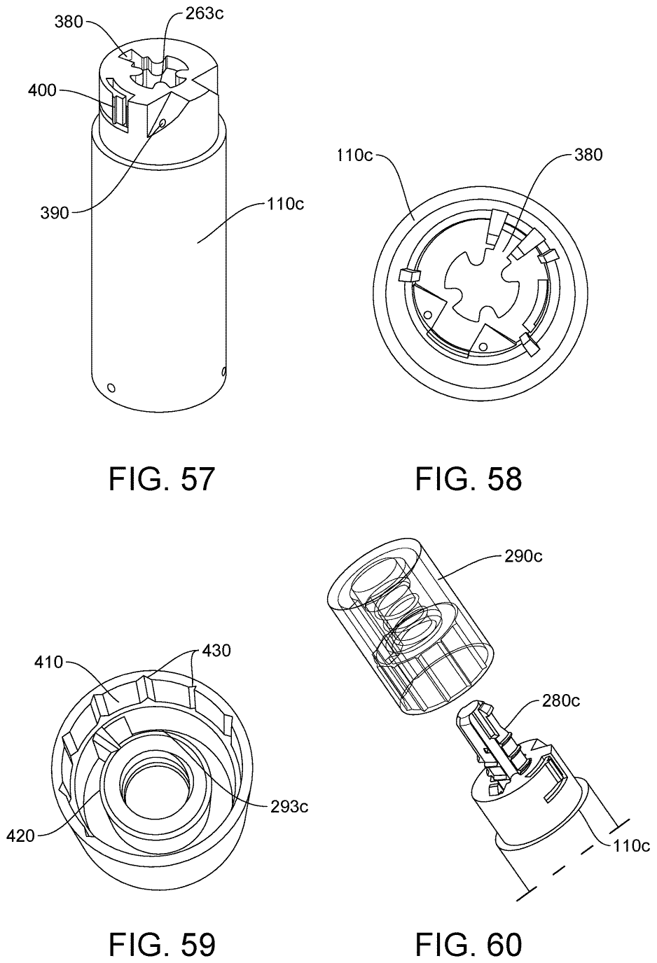

In other aspects as illustrated in FIGS. 57 to 60 (and which is relevant to not only the fourth embodiment, but also the third embodiment—i.e., embodiments of the present invention employing an actuation housing with a proximal cap, wherein the interaction between the proximal cap and the plunger define the dosing), the relationship between proximal cap 290c and actuation housing 110c may be stabilised so as to not allow free axial rotation of proximal cap 290c relative to actuation housing 110c, and therefore alteration of dose for example, during the injection procedure. One means of achieving this is a ratchet mechanism as illustrated in FIGS. 57-60, wherein a biased pawl 400 is provided on the perimeter of actuation housing 110c and proximal-distal indentations 430 are provided on the inner surface of proximal cap 290c such that, when proximal cap 290c is placed in position over actuation housing 110c (with proximal cap 290c and actuation housing 110c being a close fit with one another), biased pawl 400 interacts with indentations 430 such that sufficient force is required to overcome that interaction to rotate proximal cap 290c relative to actuation housing 110c. Other mechanisms for stabilising the relationship between proximal cap 290c and actuation housing 110c will be readily apparent to those in this field and may include, for example, a friction fit or mechanism.

In various embodiments/aspects of the injection devices according to the present invention plungers may be composed of two or more parts instead of being a single body. For example, one part of a plunger in a device according to the present invention may have a toggle and the dose setting mechanism attached thereto which a second part may be provided by the formulation chamber (for example attached to stopper). Thus, It will be understood to one having ordinary skill in the art that plunger could be construed to mean a plunger provided as one integral piece, or a mechanism having multiple components which combine to create a plunger as described in this invention.

As mentioned above, a person skilled in the art given the benefit of the above teachings will be able to derive alternative embodiments, including devising alternative actuation mechanisms which operate in the same manner using no more than materials and designs that are either already well known to those working in the field of injection devices or which would involve no more than routine workshop variations of what has been described above.

Devices according to the present invention may be used for injection of any formulation, including liquid, solid (e.g. film, implants, gels, or particle(s)), suspensions, or semi-solid or phase-shifting formulations, and may be adapted for injection of any desired injectable therapeutic to any desired tissue or organ.

According to one particular embodiment of the present invention, an injection device is provided for intravitreal injection.

Intravitreal injections have been used to administer pharmaceutically active agents to eyes to treat various eye conditions, such as macular degeneration, diabetic retinopathy, retinal vein occlusion and posterior uveitis. The active agents used are typically steroids, which may have longterm adverse systemic effects if administered systemically, and antibodies, such as anti-VEGF antibodies (such as bevacizumab, conbercept, aflibercept, pegaptanib and ranibzumab), which diffuse poorly from the blood system into eye tissues, and therefore intraocular, rather than systemic injection can be preferred in such instances.

In performing intravitreal injections, care needs to be taken to ensure no, or minimal damage to other parts of the eye, including the lens, the retina, the ciliary bodies and muscles, the suspensory ligaments, or the cornea.

The Pars plana is a discrete area of the sclera. This area is a virtually concentric ring that is located between 2 mm and 4 mm away from the limbus, which in turn is 1-2 mm wide annular transition zone between the cornea and the sclera. This area is devoid of the inner retinal layer, is almost behind ciliary body and muscle, and allows direct access to the vitreous humour, avoiding the lens, making it the preferred target for intravitreal injection.

Thus, and having reference to FIGS. 1, 2, 5, 6, 8-11, 15, 24 and 25, as well as FIGS. 35 to 43, 93 and 95, in one embodiment, an injector device according to the present invention comprises a sheath 170, 170c or 170d (either as part of a needle hub 150, 150c or 150d or as an extension of sleeve 70, 70c or 70d), wherein sheath 170, 170c or 170d has an eye-contacting surface at its distal end, or a pad 177, 177c or 172d at its distal end that acts as an eye-contacting surface. The eye-contacting surface may be padded or have a rough surface to better hold its position on the eye during injection. In further embodiments the eye-contacting surface may be provided with features such as protrusions, ridges and/or other textural elements. In yet further embodiments, the contacting surface of the distal surface of sheath may include a combination of the aforementioned features or materials. Such materials and features may assist in holding the distal surface in position (for example in the correct injection position on the eye surface for intraocular injection) during the injection procedure, and may further assist the clinician in controlling or retaining the position of the eye in a desirable position during the injection. The eye-contacting surface may have an outer perimeter that defines a circle or modified circle, said surface being annular or in the form of a perforated concave disc, discoid or 3-dimensional annulus surface, with the needle defining the centre of said circle, although other shapes may be used. Where the eye-contacting surface is surface annular or in the form of a perforated concave disc, discoid surface or 3-dimensional annulus (needle 60/64 passes through the perforation), the diameter of the circle may be between 4 to 10 mm, more preferably between 6 mm and 8 mm, so as to provide a ready measurement of 2-5, preferably 3-4 mm from the limbus to the Pars plana, to ensure injection in the vitreous body of the eye. Where other eye-contacting surface shapes are used, this should still provide at least one eye-surface contacting edge that is 2-5 mm, preferably 3-4 mm from the needle to allow for correct positioning of the needle for intravitreal injection. Where the eye-contacting surface is a pad 177, it may be made of a material that is softer or spongier, and/or is more capable of gripping the eye surface (to avoid movement of the sheath on the eye surface during the injection procedure) than the material of which sheath 170 or 170c is comprised and may, for example, be made of silicone or rubber or an absorbent material. In certain embodiments sheath 170, 170c or 170d for an intravitreal injection device according to the present invention may be substantially hollow cylindrical for most of its length, optionally with a frustoconical (narrowing) distal end. In use the eye contacting surface is placed on the sclera of the eye surface, with one edge of the eye-contacting surface being placed on the limbus (boundary of the white sclera and the cornea/iris) with the rest of the eye-contacting surface resting on the sclera (away from the cornea), thus ensuring that needle 60 or 64 enters the eye through the Pars plana region, substantially reducing the risk of injury to major structures of the eye, including the retina, lens, and ciliary body. In certain embodiments the eye-contacting surface, such as pad 177 or 177c, may have a pad 179 extending peripherally therefrom for applying pressure near the injection site after injection, to assist in stemming post wound leakage/reflux. A further advantage of pad 177 or 177c is that it introduces significant asymmetry in sheath 170 or 170c and cap 180, such that a corresponding needle hub with cap 180 on can be readily screwed into barrel connector 46 or 210 or 210c by applying axial rotational pressure on cap 180. The sheath 170 or 170c may also comprise a speculum for engaging an eyelid and keeping it away from the injection site during injection. The speculum may extend from the body of sheath 170 or 170c with its eyelid contacting means being coterminous with eye-contacting surface 177 or 177c.

To avoid damage to the retina, intravitreal injection should ideally aim to deliver formulation to the centre of the vitreous cavity. Accordingly, in one embodiment, an intravitreal injection device according to the present invention may be configured to deliver pharmaceutical formulation at a depth of approximately 4-10 mm, preferably 5-7 mm. The injection depth at which plunger 80 or 80c is released by the interaction between retention arm or toggle 120 or 120c and restrainer 130 or 130c (i.e., the injection depth at which injection is actuated) can be pre-determined based on: the size (i.e. proximal to distal length) of retention tabs 128 or 128c, and the thickness of restrainer 130 or 130c; the ‘dead’ space between the proximal surface of sleeve body 72 or 72c and the bottom surface of restrainer 130 or 130c; and the ‘dead’ space between the eye-contacting surface and the tip of needle 60 when at rest.

Injection devices according to the present invention may be disposable or be adapted to be used a number of times, either using replacement of formulation chambers or the device may be adapted for multiple injections from the same formulation chamber using replaceable, disposable needle hubs as described herein. In some embodiments, the injection device can be preassembled with formulation chamber and sterile packaged and ready for use without any further assembly. In other embodiments the injection device may be provided without needle hub (but with syringe cap in the barrel connector), and the needle hub provided separately, both sterile packaged. Certain injection devices according to the present invention are adapted for receiving standard formulation chambers or cartridges, which may be filled (including vacuum filled), sealed and sterilised in a facility skilled and experienced in such procedures, whereas the injector device and needle hub (if separate) may be manufactured at a different facility, skilled and experienced in those manufacturing procedures.

In particular aspects, injection devices according to the present invention may be fully disposable or comprise a pre-assembled disposable single use elongated housing (including formulation chamber, sleeve, needle assembly, sheath) where the pre-assembled elongated housing is separable from an actuation housing that can be re-set, such as by a mechanism as described above and illustrated in FIGS. 99-100 and 108, to allow replacement of pre-assembled elongated housings (for example, one per patient). In such embodiments, the formulation chamber may be formed integrally with the elongated housing, or it may be adapted to have syringes or cartridges inserted into it. In such aspects the needle may be formed integrally with the elongated housing by, for example, being glued or overmoulded during formation of the elongated housing. Alternatively, the elongated housing may be adapted to have a needle assembly, such as a needle hub as described above, provided separately and attached. Where the elongated housing is adapted to receive a formulation cartridge (typically pre-filled), it may be advantageous to have the needle assembly (whether detachable or not) have a spring at its proximal end to urge the formulation chamber proximally and against the plunger (to assist in priming of the needle).

In configurations where the elongated housing is adapted to receive pre-filled syringes adapted to have needle hubs attached thereto, then a spring between the syringe and the needle assembly may be replaced by a spring between the proximal end of the elongated housing and the proximal end or flange of the syringe. A spring in this proximal position would keep the syringe urged distally and in contact with the needle assembly, sealing between the two as the needle hub is screwed on. As the needle hub is positioned on the distal end of the syringe the plunger contacts the stopper, pushing it forward, resulting in priming of the needle. Because the spring in proximal position keeps the needle hub in contact with the syringe tip, priming fluid is expelled through the needle and the system is primed when the needle hub is fully positioned.

In one particular aspect of the present invention, there is provided an injection device comprising an elongated housing having a proximal end and a distal end, the proximal end being removably attached to an actuation mechanism, wherein: the elongated housing defines an inner lumen between the proximal and distal ends which includes a formulation chamber formed integrally with the elongated housing, the formulation chamber also having corresponding proximal and distal ends, and having a stopper between its proximal and distal ends for retaining formulation in the formulation chamber; the actuation mechanism having corresponding proximal and distal ends, and comprises an actuation housing with a proximal end connected to a dose delivery mechanism, and a distal end adapted for engaging the elongated housing, wherein the actuation mechanism includes a plunger, the distal end of the plunger engaging the stopper or being adapted to engage the stopper, and the plunger being in communication with a driving spring positioned between it and the proximal end of the actuation housing and which biases the plunger distally when the actuation mechanism is primed for injection, wherein the plunger is held in position against the bias by a toggle which has distal and proximal ends, and is connected to the plunger by a pivot point located between the distal and proximal ends, the toggle having an outwardly directed ledge positioned between the pivot point and the proximal end and adapted to engage an opposed inwardly directed ledge on or connected to the actuation housing to hold the plunger in a first unactuated position when the actuation mechanism is primed for injection, the proximal end of the toggle including a surface adapted to engage a complementary surface of a restrainer to hold the toggle in an unactuated position and thereby keep the outwardly directed ledge and opposed inwardly directed ledge in an engaged position, wherein the restrainer translates from an unactuated position or configuration to an actuated position or configuration when triggered, thereby allowing the toggle to deflect to an actuated position thereby uncoupling of the engagement between the outwardly directed ledge and opposed inwardly directed ledge and release of the plunger to move distally; the elongated housing comprises a sleeve which is at least adapted to slide proximally between the elongated housing and the formulation chamber and is adapted to engage the restrainer such that proximal movement of the sleeve causes proximal movement of the restrainer; the distal end of the elongated housing has a needle assembly comprising a needle that is glued or overmoulded in the distal end of the formulation chamber integrally formed with the elongated housing and comprising a sheath, the sheath being adapted to engage the distal end of the sleeve, wherein the sheath is at least proximally slidable with respect to the needle so as to unsheath the needle during insertion of said needle into a subject and so as to slide the sleeve proximally, which results in proximal movement of the restrainer from the unactuated position to the actuated position, uncoupling of the engagement between the outwardly directed ledge and opposed inwardly directed ledge, and release of the plunger to move distally; wherein the dose delivery mechanism comprises a first internal knob, a second, external dose setting knob, and a dose delivery housing adapted to sit on the proximal end of the actuation housing, wherein the proximal end of the plunger engages with the first knob via a thread with axial:longitudinal pitch X, the first knob engages with the second knob axially, but not longitudinally, and the second external dose setting knob engages with the dose delivery housing via a thread with axial:longitudinal pitch Y, wherein pitch Y is greater than pitch X, such that axial rotation of the second external dose setting knob results in the same degree of axial rotation as the first knob, but greater longitudinal travel of the second external dose setting knob compared to the first knob wherein the actuator mechanism comprises a restrainer and retainer toggle which have interacting surfaces which are configured such that the toggle returns to its unactuated position when the plunger is moved proximally against the bias of the driving spring such that the first and second surfaces are again in an engaged position, and such that the restrainer returns to its unactuated position or configuration to again restrain the toggle in its unactuated position and ready to be triggered again.

In another particular aspect of the present invention, there is provided an injection device comprising an elongated housing having a proximal end and a distal end, the proximal end being removably attached to an actuation mechanism, wherein: the elongated housing defines an inner lumen between the proximal and distal ends which includes a separately formed formulation cartridge, the formulation chamber also having corresponding proximal and distal ends, and having a stopper between its proximal and distal ends for retaining formulation in the formulation cartridge; the actuation mechanism having corresponding proximal and distal ends, and comprises an actuation housing with a proximal end connected to a dose delivery mechanism, and a distal end adapted for engaging the elongated housing, wherein the actuation mechanism includes a plunger, the distal end of the plunger engaging the stopper or being adapted to engage the stopper, and the plunger being in communication with a driving spring positioned between it and the proximal end of the actuation housing and which biases the plunger distally when the actuation mechanism is primed for injection, wherein the plunger is held in position against the bias by a toggle which has distal and proximal ends, and is connected to the plunger by a pivot point located between the distal and proximal ends, the toggle having an outwardly directed ledge positioned between the pivot point and the proximal end and adapted to engage an opposed inwardly directed ledge on or connected to the actuation housing to hold the plunger in a first unactuated position when the actuation mechanism is primed for injection, the proximal end of the toggle including a surface adapted to engage a complementary surface of a restrainer to hold the toggle in an unactuated position and thereby keep the outwardly directed ledge and opposed inwardly directed ledge in an engaged position, wherein the restrainer translates from an unactuated position or configuration to an actuated position or configuration when triggered, thereby allowing the toggle to deflect to an actuated position thereby uncoupling of the engagement between the outwardly directed ledge and opposed inwardly directed ledge and release of the plunger to move distally; the elongated housing comprises a sleeve which is at least adapted to slide proximally between the elongated housing and the formulation cartridge and is adapted to engage the restrainer such that proximal movement of the sleeve causes proximal movement of the restrainer; the distal end of the elongated housing has a needle assembly integrally formed with the elongated housing comprising a needle or needles that extend distally and proximally from the needle assembly which also comprises a sheath, the sheath being adapted to engage the distal end of the sleeve, wherein the sheath is at least proximally slidable with respect to the needle so as to unsheath the needle during insertion of the needle into a subject and so as to slide the sleeve proximally, which results in proximal movement of the restrainer from the unactuated position to the actuated position, uncoupling of the engagement between the outwardly directed ledge and opposed inwardly directed ledge, and release of the plunger to move distally, wherein the needle assembly also comprises a spring at its proximal end and adapted to push proximally against the distal end of the formulation cartridge such that attachment of the needle assembly to the elongated housing results in piercing of the formulation cartridge septum and engagement of the plunger with the stopper and positive pressure between said plunger and said needle assembly resulting in a small amount of fluid being expelled from the formulation chamber through the needle, the needle being thereby primed with formulation; wherein the dose delivery mechanism comprises a first internal knob, a second, external dose setting knob, and a dose delivery housing adapted to sit on the proximal end of the actuation housing, wherein the proximal end of the plunger engages with the first knob via a thread with axial:longitudinal pitch X, the first knob engages with the second knob axially, but not longitudinally, and the second external dose setting knob engages with the dose delivery housing via a thread with axial:longitudinal pitch Y, wherein pitch Y is greater than pitch X, such that axial rotation of the second external dose setting knob results in the same degree of axial rotation as the first knob, but greater longitudinal travel of the second external dose setting knob compared to the first knob; wherein the actuator mechanism comprises a restrainer and retainer toggle which have interacting surfaces which are configured such that the toggle returns to its unactuated position when the plunger is moved proximally against the bias of the driving spring such that the first and second surfaces are again in an engaged position, and such that the restrainer returns to its unactuated position or configuration to again restrain the toggle in its unactuated position and ready to be triggered again.

In other aspects injection devices according to the present invention may be adapted for multiple injections from the same formulation chamber, be adapted for injection of different formulations or be fully re-usable (with the exception of formulation chamber and needles). In such aspects the elongated housing needs to be adapted to have needle assemblies, such as needle hubs as described above, replaceably attached thereto or to have syringes or cartridges, such as pre-filled syringes or cartridges, replaceably inserted therein—one per injection. In certain aspects such an elongated housing may be pre-assembled with a large volume formulation chamber (which may be integral or not) and be provided with an actuation mechanism that can be re-set, and allow for multiple injections from the one formulation chamber, replacing needle hubs only between injections. In other aspects the elongated housing is adapted to replaceably receive syringes, such as pre-filled syringes (which may have their own needles) or be adapted to receive formulation cartridges and have separately provided needle assemblies, such as needle hubs as described above, removably attached thereto.

Components of the injection devices according to the present invention may be sterilizable by any method known in the art, such as by autoclaving, gamma radiation, ethylene oxide sterilization, etc. In particular, needle assemblies, including needle hubs as described herein, as well as elongated housings (and components thereof) and plungers may be sterilizable and made of appropriate materials as known in the art. For example, while not intending to be limiting, springs, housings, and even plungers, retention arms/toggles intended to be re-used repeatedly may be made from stainless steel, although most components may be made from suitable plastics, such as polycarbonate (PC), acetonitrile butadiene styrene (ABS), PC/ABS combinations, acetal (polyoxymethylene), or nylon. Certain needle hub components, such as needle caps may be made from similar plastics or also polypropylene, polyisoprene or a thermoplastic elastomer. Needle valves, such as duck valves are typically made of silicone, and filters for use in needle hubs according to the invention, as described above, may be made of paper or other suitable polymeric insoluble porous material. Formulation chambers of various sorts are typically made of glass (which may be spray or bake-coated with silicone) or thermostable plastics and comprise stoppers or septa which are typically made of a suitable rubber, such as chlorobutyl- or bromobutyl-rubbers. Where elongated housings are intended to have formulation chambers formed integrally, these may be made from, for example, cyclic olefin polymer.

Additional aspects of the invention are also described as follows:

Aspect 1. An injection device comprising an elongated housing having a proximal end and a distal end, the proximal end comprising an actuation mechanism, wherein: said elongated housing defines an inner lumen between said proximal and distal ends which is adapted to receive a formulation chamber and hold it in a fixed position relative to said actuation mechanism, said formulation chamber comprising or adapted to comprise an active agent for injection into a subject and said formulation chamber also comprising corresponding proximal and distal ends, and having stopper between its proximal and distal ends for retaining formulation in said formulation chamber and the distal end comprising a needle or being adapted to be in fluid communication with a needle. said actuation mechanism also comprises corresponding proximal and distal ends, and comprises an actuation housing with a proximal end, and a distal end adapted for engaging said elongated housing, wherein said actuation mechanism comprises a plunger, the distal end of said plunger engaging said stopper or being adapted to engage said stopper, and the proximal end of said plunger being in communication with a first spring, said spring being positioned between said plunger and the proximal end of the actuation mechanism housing and being under stress when the actuation mechanism is primed for injection, wherein said plunger is held in position against said stress by retention arm or toggle, the bias force being directed outwardly from said plunger, said retention arm or toggle having plunger engaging means, wherein said retention arm or toggle engage said plunger when the actuation mechanism is primed for injection, said retention arm or toggle being held in plunger engaging position against the bias force by restrainer which translate from a restraining position or configuration to a releasing position or configuration when triggered.

Aspect 2. The injection device of Aspect 1, wherein the restrainer translates from a restraining position or configuration to a releasing position or configuration by a mechanism activated when a needle connected to the injection device reaching a desired injection depth.

Aspect 3. The injection device of Aspect 1 or Aspect 2, wherein the elongated housing also comprises a sleeve which is at least proximally slidable with respect to the elongated housing, and which slides proximally as a needle connected to the injection device is inserted into a subject.

Aspect 4. The injection device of Aspect 3, wherein the proximal movement of the sleeve results in proximal movement of the restrainer.

Aspect 5. The injection device of Aspect 4, wherein the restrainer are in restraining position when distal and in releasing position when proximal wherein, in use, said restrainer moves proximally as said sleeve is slid proximally relative to said elongated housing, thereby releasing the retention arm or toggle to deflect away from the plunger, allowing said first spring to drive said plunger and the stopper distally.

Aspect 6. The injection device of Aspect 5, wherein at least when in use, the distal end of the injection device comprises a sheath enshrouding at least a distal portion of a needle assembly in fluid communication with said formulation chamber, said sheath being either an integral part of said sleeve or being adapted to engage the distal end of said sleeve and being adapted to slide between said elongated housing and said formulation chamber, and wherein said sheath is at least proximally slidable with respect to the needle so as to unsheath the needle during insertion of said needle into a subject and so as to move said sleeve proximally, resulting in proximal movement of the retaining means to a retention arm or toggle-releasing position.

Aspect 7. The injection device of Aspect 6, wherein said sleeve and sheath are held in a needle enshrouding position by a second spring stressed between the actuation housing and the sleeve, which is adapted to engage said second spring.

Aspect 8. The injection device of Aspect 6 wherein said sheath comprises biasing means stressed between a distal portion of the sheath and the needle assembly to ensure the sheath covers the needle tip.

Aspect 9. The injection device of any one of Aspects 6 to 8, wherein said retention arm or toggle, once released, is adapted to stop said sleeve and sheath from proximal movement once said sleeve and sheath have returned to a needle-enshrouding position.

Aspect 10. The injection device of any one of Aspects 1 to 9, wherein said retention arm or toggle comprise at least one biased arm, said at least one biased arm being connected at one end to the proximal end of said actuation mechanism housing, the other end of said at least one biased arm comprising an inwardly directed ledge adapted to engage a complementary outwardly directed ledge on said plunger, said outwardly directed ledge being optionally formed as a groove in or a ridge on said plunger.

Aspect 11. The injection device of any one of Aspects 1 to 10, wherein said actuation mechanism housing and said elongated housing are detachable from one another to allow placement, removal or replacement of formulation chamber(s) into or from said elongated housing.

Aspect 12. The injection device of Aspect 11 wherein said formulation chamber is pre-filled, optionally vacuum or voidless filled.

Aspect 13. The injection device of Aspect 12, wherein engagement of said plunger with said stopper and placement of the formulation chamber in said elongated housing and connection of said actuation housing to said elongated housing results in positive pressure within said formulation chamber or actuation mechanism.

Aspect 14. The injection device of Aspect 13, wherein when a needle is placed in fluid communication with the distal end of said formulation chamber, said positive pressure results in a small amount of fluid being expelled from the formulation chamber through said needle, the needle being thereby filled with formulation.

Aspect 15. The injection device of any one of Aspects 1 to 14, wherein the distal end of said formulation chamber is adapted to have a needle assembly attached thereto.

Aspect 16. The injection device of any one of Aspects 1 to 14, wherein the distal end of said elongated housing is adapted to have a needle assembly attached thereto and allow fluid communication between said formulation chamber and said needle.

Aspect 17. The injection device of Aspect 15 or Aspect 16 wherein said needle assembly is attached to the distal end of said formulation chamber or said elongated housing by means of a screw or a Luer lock mechanism.

Aspect 18. The injection device of any one of Aspects 1 to 14, wherein said needle forms an integral part of at least said formulation chamber.

Aspect 19. The injection device of any one of Aspects 1 to 14, wherein said needle forms an integral part of said elongated housing.

Aspect 20. The injection device of any one of Aspects 1 to 17, which comprises:
an elongated housing comprising an inner sleeve said housing and sleeve defining a lumen adapted to receive a formulation chamber;
an actuation mechanism housing detachably connectable to the proximal end of said elongated housing; and optionally
a needle assembly detachably connectable to the distal end of said elongated housing or said formulation chamber, said needle assembly comprising a sheath which is adapted to engage the distal end of said sleeve and being adapted to slide between said elongated housing and said formulation chamber, and wherein said sheath is at least proximally slidable with respect to the needle so as to unsheath the needle during insertion of said needle into a subject and so as to slide said sleeve proximally, resulting in proximal movement of said restrainer to a retention arm or toggle releasing position.

Aspect 21. The injection device of any one of Aspects 1 to 20, wherein said actuation housing comprises proximal and distal portions engaged via adjustable means, such that the distance between the proximal and distal ends of said actuation housing, and therefore the allowable travel distance of said plunger, can be set to a specified distance, thereby allowing setting of a defined volume of formulation to be dispensed on actuation of the injection device.

Aspect 22. The injection device of any one of Aspects 1 to 20, wherein said plunger has a proximal end that extends proximally beyond the actuation housing and engages with a cap via adjustable means, such that the distance between the cap and the actuation housing, and therefore the allowable travel distance of said plunger can be set to a specified distance, thereby allowing setting of a defined volume of formulation to be dispensed on actuation of the injection device.

Aspect 23. The injection device of any one of Aspects 1 to 22 which comprises a mechanism that signals successful actuation of the device via audible, tactile or visual means.

Aspect 24. The injection device of any one of Aspects 1 to 23 which is for intravitreal injection and comprises a sheath enshrouding at least a distal portion of a needle assembly in fluid communication with said formulation chamber, said sheath being either an integral part of said sleeve or being adapted to engage the distal end of said sleeve and being adapted to slide between said elongated housing and said formulation chamber, and wherein said sheath is at least proximally slidable with respect to the needle so as to unsheath the needle during insertion of said needle into a subject and so as to slide said sleeve, resulting in proximal movement of the sleeve against said restrainer and movement of said restrainer to a retention arm or toggle-releasing position, wherein said sheath has a distal eye-contacting surface.

Aspect 25. The injection device of Aspect 24, wherein said eye-contacting surface has an outer perimeter that defines a circle or modified circle, said surface being annular or in the form of a perforated concave disc, discoid or 3-dimensional annulus surface, with the needle defining the centre of said circle, and wherein the diameter of said circle is between 4 to 10 mm, optionally between 6 mm and 8 mm.

Aspect 26. The injection device of Aspect 24 or Aspect 25 wherein said sheath comprises an external pad which is substantially coterminous with said eye-contacting surface for applying pressure next to the injection site after injection.

Aspect 27. A needle hub for attachment to an injection device according to any one of Aspects 12 to 17 or 20 comprising a needle and a body, said body comprising a proximal end having the proximal end of the needle protruding proximally from it and a main body with the injection end of the needle protruding distally from it, the proximal end of said body being adapted for engagement with said elongated housing or said formulation chamber, the needle hub also comprising a sheath enshrouding the needle and main body, the proximal end of the sheath being adapted to: retain the main body of the needle assembly within the sheath; to engage the sleeve of said injection device; and to fit and slide within the elongated housing, said needle hub optionally also comprising a cap which fully enshrouds at least the distal end of said needle hub.

Aspect 28. A needle hub for attachment to an injection device according to any one of Aspects 12 to 17 or 20 comprising a main body having a proximal body and a distal body, the proximal body being adapted for engagement with the elongated housing or the formulation chamber and a proximal needle extending proximally from it, the distal body comprising a distal needle extending distally from it, the proximal and distal bodies being adapted to connect together with a filter enclosed between them and between the two needles, the needle hub also comprising a sheath enshrouding the needle and main body, the proximal end of the sheath being adapted to: retain the main body of the needle assembly within the sheath when not in use; to engage the sleeve of said injection device; and to fit and slide within the elongated housing.

Aspect 29. The needle hub of Aspect 28, wherein the proximal needle has a larger gauge than the distal needle.

Aspect 30. The needle hub of Aspect 29, wherein the distal needle has a gauge of 25 (approximately 0.5 mm outer diameter) or finer, preferably gauge 27 (approximately 0.4 mm outer diameter) or finer.

Aspect 31. The needle hub of any one of Aspects 27 to 30 wherein said sheath is substantially cylindrical for most of its length, with a frustoconical distal end.

Aspect 32. The needle hub of any one of Aspects 27 to 31 which is for intravitreal injection and has a distal eye-contacting surface.

Aspect 33. The needle hub of Aspect 32, wherein said eye-contacting surface has an outer perimeter that defines a circle or modified circle, said surface being annular or in the form of a perforated concave disc, discoid or 3-dimensional annulus surface, with the needle defining the centre of said circle, and wherein the diameter of said circle is between 4 to 10 mm, optionally between 6 mm and 8 mm.

Aspect 34. The needle hub of Aspect 32 or Aspect 33, which comprises a sheath which has an external pad which is substantially coterminous with the eye-contacting surface for applying pressure next to the injection site after injection.

Aspect 35. The needle hub of any one of Aspects 27 to 34 comprising a cap when not in use, said cap being adapted to fully enshroud at least the distal end of said needle hub, optionally also including means to ensure that said cap does not rotate axially with respect to said main body when connecting the needle hub to said injection device.

Aspect 36. A delivery device configured to deliver a medicinal agent to an area of anatomy of a patient, the delivery device having:
an elongated housing having a proximal end and a distal end and defining an inner lumen extending between the proximal end and the distal end;
at least one actuation mechanism, disposed within the inner lumen of the proximal end of the elongated housing, further having corresponding proximal and distal ends, at least one plunger, and at least one retention means configured to retain the at least one actuation mechanism in a first primed configuration, wherein the at least one retention means is in a first position corresponding to the first primed configuration wherein the at least one retention means restricts the at least one plunger from moving in at least one axis of motion, and further wherein the at least one retention means is configured to be movable to a second position corresponding to a second delivery configuration further wherein movement of the at least one plunger is no longer restricted by the at least one retention means;
at least one chamber configured to contain a medicinal agent, having corresponding proximal and distal ends;
at least one needle hub, coupled to the distal end of the elongated housing, having at least one needle configured to deliver the medicinal agent to an anatomical area of the patient, and configured to be coupled to the at least one chamber such that the at least one needle is in fluid communication with the at least one chamber, and further having at least one needle sheath, which is slidably movable with respect to an axis substantially normal to the anatomical area of a patient, wherein this movement is capable of moving the at least one needle sheath between a first non-retracted configuration and a second retracted configuration;
at least one dose selection mechanism, coupled to the proximal end of the elongated housing and to the proximal end of the at least one actuation mechanism, wherein the at least one dose selection mechanism is configured to select a desired dose of medicinal agent;
and
at least one trigger mechanism having a first unactuated position and a second actuated position, configured to trigger a transition of the at least one actuation mechanism from a first primed configuration to a second delivery configuration and further configured to be in communication at least one actuation mechanism;
wherein the transition of the at least one trigger mechanism from a first unactuated position to a second actuated position triggers the movement of the at least one retention means from its first position to its second position, triggering movement of the at least one actuation mechanism as determined by the at least one dose selection mechanism, and triggering expulsion of a correlated desired dose of medicinal agent from the at least one chamber through the at least one needle into the anatomical area of the patient.

Aspect 37. The delivery device of Aspect 36, wherein the at least one trigger mechanism is induced to move from the first unactuated position to a second actuated position by pressing the at least one needle sheath of the at least one needle hub to the anatomical area to be injected.

Aspect 38. The delivery device of Aspect 36 or 37, wherein the at least one chamber further has at least one stopper located at at least one of its proximal or distal end.

Aspect 39. The delivery device of Aspect 38, wherein the at least one stopper is disposed at both the proximal and distal ends of the at least one chamber.

Aspect 40. The delivery device of Aspect 38 or 39, wherein the at least one stopper disposed at the proximal end is coupled to the at least one plunger.

Aspect 41. The delivery device of any of Aspects 38-40 wherein the at least one needle of the needle hub is configured to extend through the at least one stopper disposed at the distal end to facilitate fluid communication between the at least one chamber and the at least one needle.

Aspect 42. The delivery device of Aspect 41, wherein the needle hub comprises at least two needles.

Aspect 43. The delivery device of Aspect 36, wherein the dose selection mechanism is configured to select a distance between the at least one dose selection mechanism and a housing, thereby providing a limit on the distance traveled by the at least one plunger of the actuation mechanism to deliver a correlated dose of medicinal agent.

Aspect 44. The delivery device of any of claims 36-43, wherein the dose selection mechanism is configured to select a discrete dose quantity.

Aspect 45. The delivery device of Aspect 44, wherein the at least one dose selection mechanism further comprises:
at least one knob having an interior surface and at least one recess disposed on said interior surface and configured to be rotatable about a longitudinal axis of the delivery device; and
at least one housing coupled to the at least one knob, having at least one ledge positioned distally relative to an inferior aspect of the at least one knob, and further having least one finger configured to fit within a space defined by the interior surface of the at least one knob, wherein the at least one finger further has at least one protrusion corresponding to a set distance from the at least one ledge and configured to be received by the at least one recess of the at least one knob;
and
wherein the at least one knob is configured to be rotated until the at least one finger of the housing is received in the at least one recess of the at least one knob, which additionally moves the knob in an axial direction away from the at least one ledge of the at least one housing and creating a distance between the inferior aspect of the knob and the at least one ledge of the housing corresponding to a dose of medicinal agent to be delivered.

Aspect 46. The delivery device of any of Aspects 36-45, wherein the at least one actuation mechanism further comprises:
at least one needle sheath, which is slidably movable with respect to an axis substantially normal to the anatomical area of a patient, this slidable movement capable of moving the at least one needle sheath between a first non-retracted configuration and a second retracted configuration;
at least one barrel sheath having a proximal end and a distal end, wherein at least one spring is disposed at the proximal end and coupled to the at least one needle sheath at the distal end, such that the movement of needle sheath from its first non-retracted configuration to its second retracted configuration induces a corresponding movement of the at least one barrel sheath, thereby compressing the at least one spring of the at least one barrel spring;
at least one plunger having a proximal end and a distal end, having at least one spring disposed adjacent to a collar disposed at the proximal end of the at least one plunger and extending into an at least one chamber of a delivery device at its distal end, the at least one plunger and the at least one chamber further extending through the at least one barrel sheath;
at least one retention arm, coupled to the at least one plunger at a point in between its proximal end and distal end, having a first unactuated position and a second actuated position; and
at least one locking ring coupled to the at least one plunger such that it is slidably movable along an axis substantially normal to the anatomical area of a patient, having a first position configured to restrict movement of the at least one retention arm, thereby retaining the at least one retention arm in its first unactuated position, and further configured to contact the proximal end of the at least one barrel sheath, wherein such contact slidably moves the at least one locking ring into a second position such that it bears against the collar disposed on the proximal end of the at least one plunger such that the at least one spring disposed at the proximal end of the at least one plunger is compressed, and further wherein movement of the at least one retention arm is unrestricted such that it can move into its second actuated position; and Wherein the movement of the at least one retention arm from its first unactuated position to its second actuated position allows the at least one spring of the proximal end of the at least one plunger to decompress, subjecting the collar of the at least one plunger to a force, thereby moving the distal end of the at least one plunger through the chamber to expel the medicinal agent from the delivery device.

Aspect 47. The delivery device of Aspects 36-46, wherein the dose selection mechanism further incorporates at least one indicator configured to indicate the desired dose.

Aspect 48. The delivery device of Aspect 47, wherein the at least one indicator is at least one of a visual indicator of the dose disposed on an exterior portion of the delivery device, a haptic indicator, an auditory indicator, or an electronic signal configured to interface with an application on a separate electronic device.

Aspect 49. The delivery device of any of Aspects 36-48, wherein the needle sheath is unable to be retracted again after a dose is delivered.

Aspect 50. The delivery device of any of Aspects 36-49, wherein the needle hub is configured to be removably attached to the housing.

Aspect 51. The delivery device of any of Aspects 36-50, wherein the at least one chamber is a separable cartridge.

Aspect 52. The delivery device of any of Aspects 36-51, wherein the delivery device is configured to be used with higher viscosity fluids.

Aspect 53. The delivery device of any of claims 36-52, wherein the device is configured for use in ophthalmological procedures.

Aspect 54. A delivery device configured to deliver a medicinal agent to an area of anatomy of a patient, the delivery device having:
an elongated housing having a proximal end and a distal end and defining an inner lumen extending between the proximal end and the distal end;
at least one chamber configured to contain a medicinal agent, disposed within the inner lumen of the elongated housing having corresponding proximal and distal ends;
at least one needle hub, coupled to the distal end of the elongated housing, having at least one needle configured to deliver the medicinal agent to the anatomical area of the patient, and configured to be coupled to the at least one chamber such that the at least one needle is in fluid communication with the at least one chamber, and further having at least one needle sheath, which is slidably movable with respect to an axis substantially normal to an anatomical area of a patient, wherein this movement is capable of moving the at least one needle sheath between a first non-retracted configuration and a second retracted configuration;
at least one barrel sheath disposed within the inner lumen of the elongated housing and around the at least one chamber, having a proximal end and a distal end, wherein at least one spring is disposed at the proximal end and coupled to the at least one needle sheath at the distal end, such that the movement of needle sheath from its first non-retracted configuration to its second retracted configuration induces a corresponding axial movement of the at least one barrel sheath, thereby compressing the at least one spring of the at least one barrel spring;
at least one plunger having a proximal end and a distal end, having at least one spring positioned adjacent to a collar disposed at the proximal end of the at least one plunger and extending into an at least one chamber of a delivery device at its distal end, the at least one plunger and the at least one chamber further extending through the at least one barrel sheath;
at least one retention arm, coupled to the at least one plunger at a point in between its proximal end and distal end, having a first unactuated position and a second actuated position;
at least one locking ring coupled to the at least one plunger such that it is slidably movable along an axis substantially normal to the anatomical area of a patient, having a first position configured to restrict movement of the at least one retention arm, thereby retaining the at least one retention arm in its first unactuated position, and further configured to contact the proximal end of the at least one barrel sheath, wherein such contact slidably moves the at least one locking ring into a second position such that it bears against the collar disposed on the proximal end of the at least one plunger such that the at least one spring disposed at the proximal end of the at least one plunger is compressed, and further wherein movement of the at least one retention arm is unrestricted such that it can move into its second actuated position;
at least one knob coupled to the proximal end of the at least one plunger having an interior surface and at least one recess disposed on said interior surface and configured to be rotatable about a longitudinal axis of the delivery device;
at least one dosing housing coupled to the at least one knob and disposed about the at least one plunger, having at least one ledge positioned distally relative to an inferior aspect of the at least one knob;
wherein the at least one knob is configured such that, when rotated, it additionally moves the knob in an axial direction away from the at least one ledge of the at least one dosing housing and creates a distance between the inferior aspect of the knob and the at least one ledge of the dosing housing corresponding to a dose of medicinal agent to be delivered; and further wherein the movement of the at least one retention arm from its first unactuated position to its second actuated position allows the at least one spring of the proximal end of the at least one plunger to decompress, subjecting the collar of the at least one plunger to a force, thereby moving the distal end of the at least one plunger through the chamber a distance corresponding to the distance between the at least one ledge of the at least one housing and the inferior edge of the at least one knob to expel a corresponding dose of the medicinal agent through the at least one needle into the area of anatomy of the patient.

Aspect 55. The delivery device of Aspect 54, wherein the at least one chamber further has at least one stopper located at at least one of its proximal or distal end.

Aspect 56. The delivery device of Aspect 55, wherein the at least one stopper is disposed at both the proximal and distal ends of the at least one chamber.

Aspect 57. The delivery device of Aspect 55 or 56, wherein the at least one stopper disposed at the proximal end is coupled to the at least one plunger.

Aspect 58. The delivery device of any of Aspects 56-57 wherein the at least one needle of the needle hub is configured to extend through the at least one stopper disposed at the distal end to facilitate fluid communication between the at least one chamber and the at least one needle.

Aspect 59. The delivery device of Aspect 58, wherein the needle hub comprises at least two needles.

Aspect 60. The delivery device of any of Aspects 54-59, wherein the delivery device is configured to select a discrete dose quantity.

Aspect 61. The delivery device of Aspect 60, wherein the interior surface of the at least one knob has at least one recess, and the at least one housing has at least one finger configured to fit within a space defined by the interior surface of the at least one knob wherein the at least one finger further has at least one protrusion corresponding to a set distance from the at least one ledge and configured to be received by the at least one recess of the at least one knob; and wherein the at least one knob is configured to be rotated until the at least one finger of the housing is received in the at least one recess of the at least one knob, which additionally moves the knob in an axial direction away from the at least one ledge of the at least one housing and creating a distance between the inferior edge of the knob and the at least one ledge of the housing corresponding to a dose of medicinal agent to be delivered.

Aspect 62. The delivery device of Aspects 54-61, wherein the delivery device further incorporates at least one indicator configured to indicate the desired dose.

Aspect 63. The delivery device of Aspects 54-62, wherein the at least one indicator is at least one of a visual indicator of the dose disposed on an exterior portion of the delivery device, a haptic indicator, an auditory indicator, or an electronic signal configured to interface with an application on a separate electronic device.

Aspect 64. The delivery device of any of Aspects 54-63, wherein the needle sheath is unable to be retracted again after a dose is delivered.

Aspect 65. The delivery device of any of Aspects 54-64, wherein the needle hub is configured to be removably attached to the housing.

Aspect 66. The delivery device of any of Aspects 54-65, wherein the at least one chamber is a separable cartridge.

Aspect 67. The delivery device of any of Aspects 54-66, wherein the delivery device is configured to be used with higher viscosity fluids.

Aspect 68. The delivery device of any of Aspects 54-67, wherein the device is configured for use in ophthalmological procedures.

Aspect 69. An actuation mechanism configured to deliver a selected dose of a medicinal agent from at least one chamber of a delivery device, through a delivery device to an anatomical area of a patient, the actuation mechanism comprising:
at least one needle sheath, which is slidably movable with respect to an axis substantially normal to the anatomical area of a patient, this slidable movement capable of moving the at least one needle sheath between a first non-retracted configuration and a second retracted configuration;
at least one barrel sheath having a proximal end and a distal end, wherein at least one spring is disposed at the proximal end and coupled to the at least one needle sheath at the distal end, such that the movement of needle sheath from its first non-retracted configuration to its second retracted configuration induces a corresponding movement of the at least one barrel sheath, thereby compressing the at least one spring of the at least one barrel spring;
at least one plunger having a proximal end and a distal end, having at least one spring disposed adjacent to a collar disposed at the proximal end of the at least one plunger and extending into an at least one chamber of a delivery device at its distal end, the at least one plunger and the at least one chamber further extending through the at least one barrel sheath;
at least one retention arm, coupled to the at least one plunger at a point in between its proximal end and distal end, having a first unactuated position and a second actuated position;
and
at least one locking ring coupled to the at least one plunger such that it is slidably movable along an axis substantially normal to the anatomical area of a patient, having a first position configured to restrict movement of the at least one retention arm, thereby retaining the at least one retention arm in its first unactuated position, and further configured to contact the proximal end of the at least one barrel sheath, wherein such contact slidably moves the at least one locking ring into a second position such that it bears against the collar disposed on the proximal end of the at least one plunger such that the at least one spring disposed at the proximal end of the at least one plunger is compressed, and further wherein movement of the at least one retention arm is unrestricted such that it can move into its second actuated position;
wherein the movement of the at least one retention arm from its first unactuated position to its second actuated position allows the at least one spring of the proximal end of the at least one plunger to decompress, subjecting the collar of the at least one plunger to a force, thereby moving the distal end of the at least one plunger through the chamber to expel the medicinal agent from the delivery device.

Aspect 70. A dosing mechanism configured to select an appropriate dose of medicinal agent for delivery of a medicinal agent to an area of anatomy of a patient by a delivery device, the dosing mechanism comprising:
at least one knob having an interior surface and at least one recess disposed on said interior surface and configured to be rotatable about a longitudinal axis of the delivery device; and
at least one housing coupled to the at least one knob, having at least one ledge positioned distally relative to an inferior aspect of the at least one knob, and further having least one finger configured to fit within a space defined by the interior surface of the at least one knob, wherein the at least one finger further has at least one protrusion corresponding to a set distance from the at least one ledge and configured to be received by the at least one recess of the at least one knob;
wherein the at least one knob is configured to be rotated until the at least one finger of the housing is received in the at least one recess of the at least one knob, which additionally moves the knob in an axial direction away from the at least one ledge of the at least one housing and creating a distance between the inferior aspect of the knob and the at least one ledge of the housing corresponding to a dose of medicinal agent to be delivered.

Aspect 71. A system for administering delivery of a medicinal agent to a patient, the system comprising:
a delivery device according to any of Aspects 1-70;
an electronic device; and
an application configured to track and store information related to the delivery of care.

Aspect 72. A method of delivering a dose of a medicinal agent to an area of anatomy of a patient using a delivery device, the method comprising the following steps:
a. Removing a needle cover from the delivery device;
b. Selecting a dose of the medicinal agent by rotating a knob positioned at the proximal end of the delivery device, thereby creating a distance between the knob and a ledge disposed on the housing of the delivery device;
c. Positioning a needle sheath adjacent to a surface of the area of anatomy of the patient;
d. Depressing the delivery device such that the needle sheath retracts into the body of the delivery device, exposing the needle;
e. Inserting the needle a depth determined by the retraction of the needle sheath into the body of the delivery device;
f. Waiting for the device to deliver the medicinal agent, using the diminishing distance between the knob and the ledge as an indicator of progress;
g. Removing the needle from the anatomy of the patient, thereby allowing the needle sheath to return to its original, non-retracted position.

Preferred forms of the present invention will now be described, by way of example only, with reference to the following examples, including comparative data, and which are not to be taken to be limiting to the scope or spirit of the invention in any way.

### EXAMPLES

### Example 1—Methods and Materials

For dose delivery accuracy and dose triggering force experiments data were derived by filling formulation cartridges with water or calibrated oil.

Injection devices according to the fifth embodiment, with delivery dose mechanisms as illustrated in FIG. 98, were used for dose delivery accuracy experiments, with three variants—IVAI 5.1, 5.2 and 5.3—which had differences in the thread between the inner knob and plunger such that IVAI 5.1 required one full turnoff the knob to deliver 100 μL, IVAI 5.2 required two full, and IVAI 5.3 required 2 full turns to deliver 100 μL turns of the knob to deliver 20 μL. Driving springs for all devices were the same (Century Springs A89, 35.05 mm in length, 1.61 N per mm) and provided a force of 34.7 N when assembled.

Injection devices according to the fifth embodiment with either dose delivery mechanisms as illustrated in FIG. 98 or as illustrated in FIG. 71 were used for the triggering force experiments.

The devices were assembled and the needles primed when the needle hubs were attached. Desired doses were then selected by turning external knob 500f to the appropriate point. When ready for testing, devices were then placed into the upper jaw of a Universal (Mechanical) Testing Machine (UTM), and pre-weighed cup receptacles placed in the lower jaw. Devices were then brought down at a constant speed until the needle sheath contact the solid lid of the cup, with the needle aligned to protrude through a hole in the lid. The UTM continued to push the needle hub into the lid as a clinician would push the device into the sclera of a patient until devices were triggered, at which time the fluid is expelled from the cartridge into the cup, through the needles. The upper jaw with devices then moved up away from cup, allowing the needle sheath to cover the distal needle once more and result in “lock out” (whereby the sheath and sleeve could no longer slide proximally). In a further test devices were brought back down to verify successful “lock out”.

The weight of the fluid and the cup post injection was determined, and subtracting the pre-injection weight of the cup allowed determination of the dose delivered and accuracy, relative to what was selected on the external knob. The upper jaw of the UTM has a force sensor that can measure the force applied to the device during the entire travel of the device in the UTM, allowing identification of the moment of triggering, and force applied at that time. The results obtained are a good representation of the results that would be obtained by a user, as the user has no input into the delivery of the fluid (other than dose setting which is part of this test protocol) and force with which the device activates as this is all automated.

### Example 2—Dose Accuracy

The results obtained are shown in Table 1.

TABLE 1

| | Injector and Formulation | | | | |
|---|---|---|---|---|---|
| | IVAI | IVAI | IVAI | IVAI | IVAI |
| | 5.3 | 5.1 | 5.1 | 5.2 | 5.2 |
| | 100 cP | 1 cP | 100 cP | 1 cP | 100 cP |
| Dose Set Target(μL) | 10 | 50 | 50 | 50 | 50 |
| Fluid viscosity | 100 cP | 1 cP | 100 cP | 1 cP | 100 cP |
| Average dose delivered (μL) | 10.45 | 51.35 | 53.38 | 51.00 | 51.17 |
| Standard deviation (μL) | 0.56 | 1.60 | 2.79 | 0.14 | 1.00 |

By comparison, values which can be found in the literature show significantly poorer dose accuracy and consistency. Tables 2 to 4 show data from three different reports on injection reproducibility: Goldberg R. A. (Charters L, Ophthalmology Times, 13 Nov. 2021, reporting on presentation by R. A. Goldberg at the American Academy of Ophthalmology 2021 annual meeting in New Orleans); Dingerkus et al (Dingerkus V L S et al (2002) Sci Rep. 12(1):18136); and Guest et al (Guest J M et al (2022), Intl Retina Vitreous, 8(1):27).

TABLE 2

| | Injector and Formulation | | |
|---|---|---|---|
| | PFS | PFS | PFS |
| | Goldberg-1 | Goldberg-2 | Goldberg-3 |
| | (Base to | (Base to | (Tip to |
| | Prox Edge) | Dist Edge) | Dist Edge) |
| Dose Set Target (μL) | 50 | 50 | 50 |
| Fluid viscosity | 1 cP | 1 cP | 1 cP |
| | (Purified water) | (Purified water) | (Purified water) |
| Average dose | 54.8 | 78.9 | 109.7 |
| delivered (μL) | | | |
| Standard deviation | 5.7 | 5.7 | 3.4 |
| (μL) | | | |

TABLE 3

| | Injector and Formulation | | | |
|---|---|---|---|---|
| | PFS | PFS | PFS | PFS |
| | Dingerkus-1 | Dingerkus-2 | Dingerkus-3 | Dingerkus-4 |
| | (NVnP) | (NVwP) | (HVnP) | (HVwP) |
| Dose Set | 50 | 50 | 50 | 50 |
| Target (μL) | | | | |
| Fluid viscosity | Aflibercept | Aflibercept | Aflibercept | Aflibercept |
| | (ca. 12 cP) | (ca. 12 cP) | (ca. 12 cP) | (ca. 12 cP) |
| Average dose | 56.06 | 70.69 | 74.22 | 81.63 |
| delivered (μL) | | | | |
| Standard | 10.32 | 4.56 | 7.41 | 3.67 |
| deviation (μL) | | | | |

The Goldberg-1, 2, and 3 values are results for using a Prefilled Syringe (PFS) in different ways, where they show that depending on how you align the plunger/stopper with the dose mark on the glass, can greatly affect your dose accuracy. Dingerkus-1, 2, 3 and 4 values were for similar tests, but also show how pressing hard on the plunger/stopper at the end of the injection also affects the dose accuracy. Table 4 shows values extrapolated from the Guest publication for pre-filled syringes (PFS), Luer-lock syringes, and similar devices.

TABLE 4

| | Injector and Formulation | | |
|---|---|---|---|
| | | Luer Syringe - | Similar |
| | | Guest | Devices |
| | PFS - Guest | BD Luer | MDS |
| | PFS Guest | Syringe Guest | Congruence |
| Dose Set Target (μL) | 50 | 50 | 50 |
| Fluid viscosity | Aflibercept | Aflibercept | N/A |
| | (ca. 12 cP) | (ca. 12 cP) | |
| Average dose delivered | 74.22 | 53.42 | N/A |
| (μL) | | | |
| Standard deviation (μL) | 15.87 | 4.61 | 2.5 |

As can be seen from the data, dose accuracy and variability (standard deviation) for the injectors according to the present invention is significantly better than for the devices tested in the Goldberg, Dingerkus and Guest articles.

### Example 3—Trigger Force

Average trigger forces for Injection devices according to the fifth embodiment when dispensing water (1 cP viscosity) or calibrated oil (100 cP viscosity) with dose delivery mechanisms as illustrated in FIG. 98 were 1.90 N (with a standard deviation of 0.39 N), with little difference between the triggering forces when delivering water or calibrated oil. Average trigger forces for Injection devices according to the fifth embodiment when dispensing water (1 cP viscosity) or calibrated oil (100 cP viscosity) with dose delivery mechanisms as illustrated in FIG. 71 (and having the same driving spring as IVAI 5.1, 5.2 and 5.3) were also very low, being 1.01 N for delivery of water (1 cP viscosity) and 1.29 N for delivery of calibrated oil (100 cP viscosity).

It will be appreciated that, although specific embodiments of the invention have been described herein for the purpose of illustration, various modifications may be made without deviating from the spirit and scope of the invention as defined in the following claims.

1-180. (canceled)

181. An injection device comprising an elongated housing having a proximal end and a distal end, the proximal end comprising an actuation mechanism, wherein:
said elongated housing defines an inner lumen between said proximal and distal ends which is adapted to receive and hold a formulation chamber relative to said actuation mechanism, said formulation chamber also having corresponding proximal and distal ends, and having a stopper between its proximal and distal ends for retaining formulation in said formulation chamber;
said actuation mechanism having corresponding proximal and distal ends, and comprises an actuation housing with a proximal end, and a distal end adapted for engaging said elongated housing, wherein said actuation mechanism includes a plunger, the distal end of said plunger engaging said stopper or being adapted to engage said stopper, said plunger being in
communication with at least one driving spring which biases the plunger distally when the actuation mechanism is primed for injection,
wherein said plunger is held in position against said bias by at least one retention arm or toggle having at least one first surface adapted to engage an opposed complementary second surface to hold said plunger in a first unactuated position when the actuation mechanism is primed for injection, the first and second surfaces being held in an engaged position by a restrainer which translates from an unactuated position or configuration to an actuated position or configuration when triggered, thereby allowing uncoupling of the engagement between the first and second surfaces and release of the plunger to move distally,
wherein the at least one retention arm or toggle further has distal and proximal ends, and further wherein the at least one retention arm or toggle is connected to the plunger by a pivot point located between said distal and proximal ends, and wherein said first surface of the at least one retention arm or toggle is in the form of an outwardly directed ledge adapted to engage an opposed complementary second surface in the form of an inwardly directed ledge on or connected to the actuation housing, the proximal end of the toggle including a surface adapted to engage a complementary surface of the restrainer.

182. The injection device of claim 181, wherein the restrainer translates from the unactuated position or configuration to the actuated position or configuration by a trigger mechanism activated when a needle connected to the injection device reaches a desired injection depth.

183. The injection device of claim 181, wherein the elongated housing includes a sleeve which is at least proximally slidable between the elongated housing and the formulation chamber and is adapted to engage the restrainer such that proximal movement of the sleeve causes proximal movement of the restrainer, and wherein operatively, insertion of a needle connected to the injection device into a surface results in the sleeve sliding proximally and, as a result, the restrainer moves proximally to the actuated position or configuration.

184. The injection device of claim 183, wherein the restrainer is in the unactuated position when distal and in the actuated position when proximal, so that operatively, the restrainer moves proximally as the sleeve is slid proximally relative to the elongated housing, thereby releasing the retention arm or toggle to uncouple the first and second surfaces allowing said at least one driving spring to drive the plunger and the stopper distally.

185. The injection device of claim 181, wherein the outwardly directed ledge is positioned between the pivot point and the proximal end of the toggle.

186. The injection device of claim 181, wherein the triggering force required to allow uncoupling of the engagement between the first and second surfaces and release of the plunger to move distally is inversely proportional to a value a/b, wherein a is the distance between the proximal end of the toggle and the pivot point and b is the distance between the first surface and the pivot point, allowing regulation of the triggering force by regulation of the value a/b.

187. The injection device of claim 186, for use in an injection indicating low triggering force and configured such that b is substantially smaller than a.

188. The injection device of claim 186, wherein the value of a divided by b is greater than or equal to 1.

189. The injection device of claim 181, wherein the elongated housing comprises a sleeve which is at least proximally slidable between the elongated housing and the formulation chamber and is adapted to engage the restrainer such that proximal movement of the sleeve causes proximal movement of the restrainer and wherein, operatively, the distal end of the elongated housing comprises a needle assembly having a sheath enshrouding at least a distal portion of a needle in fluid communication with said formulation chamber, wherein the sheath is at least proximally slidable with respect to the needle so as to unsheath the needle during insertion of the needle into a subject, resulting in proximal sliding of the sleeve and proximal movement of the restrainer to the actuated position.

190. The injection device of claim 189, wherein the retention arm or toggle in the actuated position is adapted to prevent the sleeve and sheath from proximal movement once the sleeve and sheath have returned to a needle-enshrouding position.

191. An injection device according to claim 181, wherein:
the elongated housing comprises a sleeve which is at least adapted to slide proximally between the elongated housing and the formulation chamber and is adapted to engage the restrainer such that proximal movement of the sleeve causes proximal movement of the restrainer;
the distal end of said elongated housing is connected to or is adapted to be connected to a needle assembly comprising a sheath, wherein said sheath is at least proximally slidable with respect to the needle so as to unsheath the needle during insertion of said needle into a subject and so as to slide said sleeve proximally, which results in proximal movement of said restrainer from the first unactuated position to the second actuated position;
the plunger is held in position against said stress by a toggle with distal and proximal ends and connected to the plunger by a pivot point located between said distal and proximal ends and wherein said first surface is in the form of an outwardly directed ledge positioned between the pivot point and the proximal end and adapted to engage said opposed complementary second surface provided in the form of a complementary inwardly directed ledge on or connected to the actuation housing, the proximal end of the toggle comprising a surface adapted to engage a complementary surface of the restrainer when in the unactuated position.

192. The injection device of claim 189, wherein the sheath is an integral part of the sleeve.

193. The injection device of claim 191, wherein the toggle is biased towards the actuated position to ensure it completes its travel.

194. The injection device of claim 189, wherein the sleeve and sheath are held in a needle enshrouding position by at least one sleeve spring stressed against a proximal surface of the sleeve which is adapted to engage said second spring.

195. The injection device of claim 189, wherein a spring is stressed between a distal portion of the sheath and the needle assembly, to ensure the sheath covers the needle tip.

196. The injection device of claim 189, wherein the sheath comprises a distal anatomy contacting surface which assists in positioning the injection device and holding it in position.

197. The injection device of claim 181, wherein said actuation mechanism housing and the elongated housing are detachable from one another to allow placement, removal or replacement of formulation chamber(s) into or from said elongated housing.

198. The injection device of claim 181, wherein said actuation mechanism housing and the elongated housing are detachable from one another to allow replacement of the elongated housing and formulation chamber after injection with a new elongated housing pre-assembled with fresh formulation chamber.

199. The injection device of claim 181, wherein the distal end of the elongated housing comprises a needle assembly as an integral part.

200. The injection device of claim 181, wherein the distal end of the elongated housing is adapted to have a needle assembly removably attached thereto, either through direct attachment to the formulation chamber, or attachment to the elongated housing in a manner which brings the needle assembly into fluid communication with the formulation chamber.

201. The injection device of claim 200, wherein the connection of the needle assembly to the elongated housing urges the formulation chamber proximally, thereby creating a positive pressure within the formulation chamber, and further wherein when the needle assembly is placed in fluid communication with the distal end of said formulation chamber, said positive pressure results in a small amount of fluid being expelled from the formulation chamber through said needle, the needle being thereby primed with formulation.

202. The injection device of claim 200, wherein the needle assembly comprises a spring at its proximal end and adapted to push proximally against the distal end of the formulation chamber such that attachment of the needle assembly to the elongated housing results in engagement of the plunger with the stopper and positive pressure between said plunger and said needle assembly resulting in a small amount of fluid being expelled from the formulation chamber through said needle, the needle being thereby primed with formulation.

203. The injection device of claim 181, wherein a dosing mechanism is adjustably coupled to the plunger at the proximal end of the actuation housing, such that a distance between the dosing mechanism and the actuation housing, and therefore an allowable travel distance of the plunger, can be set to a specified value, so that a selected volume of formulation may be operatively dispensed.

204. The injection device of claim 203, wherein the plunger has a proximal end that extends proximally beyond the actuation housing and engages with a cap or knob such that a distance between the cap or knob and the actuation housing may be adjusted, and therefore an allowable travel distance of said plunger is controlled, so that operatively adjustment of the cap allows delivery of a selected volume of formulation.

205. The injection device of claim 203, wherein the dosing mechanism comprises a first internal knob, a second, external dose setting knob, and a dose delivery housing adapted to sit on the proximal end of the actuation housing, wherein the proximal end of the plunger engages with the first knob via a thread with axial:longitudinal pitch X, the first knob engages with the second knob axially, but not longitudinally, and the second external dose setting knob engages with the dose delivery housing via a thread with axial:longitudinal pitch Y, wherein pitch Y is greater than pitch X, such that axial rotation of the second external dose setting knob results in the same degree of axial rotation as the first knob, but greater longitudinal travel of the second external dose setting knob compared to the first knob.

206. The injection device of claim 203, wherein the coupling of the dosing mechanism to the plunger is configured in a manner which ensures the dosing mechanism is set to a ‘0’ setting when assembled and ready for injection.

207. The injection device of claim 181, which comprises a mechanism that signals one or more of actuation of the device, completion of the injection, confirmation of dose setting, or lock out of the device after injection and wherein the signal is auditory, haptic, visual, or electronic, or is optionally configured to interface with one or more devices or applications.

208. The device of claim 181, which is adapted for single use.

209. The device of claim 181, which is adapted for use on multiple patients, for multiple injections, or for injection of different formulations.

210. The device of claim 209, which comprises a dosing mechanism that is configured to be re-set to a ‘0’ setting after each injection.

211. A method for operating an injection device, the injection device having an elongated housing having a proximal end and a distal end, the proximal end having an actuation mechanism and the distal end having or being adapted to have a needle attached thereto, the injection device further including a formulation chamber including a stopper,
the actuation mechanism including a plunger, the distal end of the plunger being adapted to engage the stopper, the plunger being biased towards the distal end when the actuation mechanism is primed for injection, the plunger being held in position against the bias by a retention arm or toggle, so as to hold the plunger in an unactuated position when the actuation mechanism is primed for injection, the plunger being held in an engaged position by a restrainer which is adapted to translate from an unactuated position or configuration to an actuated position or configuration when triggered, wherein the device further includes a needle, a selectively adjustable proximal cap, alignment or positioning structures provided distally on the injection device, a movable distal projection, a dose delivered indicator
the method comprising selecting a desired dose by adjusting the proximal cap, priming the device such that formulation fills the needle, bringing the device into an operative position, including positioning the device adjacent to a desired anatomical location and engaging positioning or alignment structures relative to specific features of the anatomy of the patient; triggering the restrainer to release the plunger from the engaged position by engaging the distal projection with a surface of a patient, the plunger then moving distally to deliver formulation from the formulation chamber; activating the dose delivered indicator after the restrainer is triggered and the dose has been delivered to the anatomy of the patient.

212. The method of claim 211, including the step of re-setting the device for re-use after delivery of formulation.
